(19) United States
(12) Patent Application Publication
(10) Pub. No.: US 2026/0218257 A1
(43) Pub. Date: Jul. 30, 2026

(54) ENZYMES, POLYNUCLEOTIDES ENCODING SAME, AND METHODS OF USING SAME FOR PRODUCING TRYPTAMINES

(71) Applicant: YEDA RESEARCH AND DEVELOPMENT CO. LTD., Rehovot (IL)

(72) Inventors: Asaph AHARONI, Tel-Aviv (IL); Janka Isabela HOFER, Rehovot (IL); Paula BERMAN, Rehovot (IL); Herschel MEHLMAN, Rehovot (IL); Sarel-Jacob FLEISHMAN, Rehovot (IL); Olga KHERSONSKY, Rehovot (IL)

(21) Appl. No.: 19/462,501

(22) Filed: Jan. 28, 2026

Related U.S. Application Data
(60) Provisional application No. 63/750,794, filed on Jan. 29, 2025.
(60) Provisional application No. 63/812,253, filed on May 27, 2025.

(51) Int. Cl. C12P 17/10; C12N 9/02; C12N 9/10; C12N 9/12; C12N 9/88

(52) U.S. Cl. CPC C12P 17/10; C12N 9/0071; C12N 9/0083; C12N 9/1007; C12N 9/1205; C12N 9/88; C12Y 401/01028

(57) ABSTRACT

The present invention provides artificial DNA molecules, polypeptides encoded by same, cells including same, and methods of using same, such as for the synthesis of tryptamine and/or at least one metabolite thereof.

### CROSS-REFERENCE TO RELATED APPLICATIONS

This application claims the benefit of priority of U.S. Provisional Patent Application No. 63/750,794 entitled “ENZYMES, POLYNUCLEOTIDES ENCODING SAME, AND METHODS OF USING SAME FOR PRODUCING TRYPTAMINES”, filed Jan. 29, 2025, and of U.S. Provisional Patent Application No. 63/812,253 entitled “ENZYMES, POLYNUCLEOTIDES ENCODING SAME, AND METHODS OF USING SAME FOR PRODUCING TRYPTAMINES”, filed May 27, 2025. The content of both applications are incorporated herein by reference in their entirety.

### REFERENCE TO AN ELECTRONIC SEQUENCE LISTING

The contents of the electronic sequence listing (YEDA-P-064-US.xml; size: 39,517 bytes; and date of creation: Jan. 27, 2026) is herein incorporated by reference in its entirety.

### FIELD OF INVENTION

The present invention relates to, inter alia, the use of enzymes including polynucleotides encoding the same, in a method for synthesizing tryptamines.

### BACKGROUND

For thousands of years, psychedelic substances have been used by indigenous cultures as entheogens in rituals intended to induce altered states of consciousness for spiritual and therapeutic purposes. Psilocybin-containing mushrooms were central to ancient Aztec ceremonies, while N,N-dimethyltryptamine (DMT), the primary psychoactive component of ayahuasca, has long been used in traditional Amazonian rituals. This ceremonial brew combines Psychotria viridis (a natural source of DMT) with Banisteriopsis caapi, which provides β-carboline monoamine oxidase inhibitors that render DMT orally active. Similarly, 5-methoxy-N,N-dimethyltryptamine (5-MeO-DMT), found in the secretion of the Sonoran Desert toad (Incilius alvarius) and in several plant species, is thought to have been used ceremonially by indigenous groups in northern Mexico (3). These traditions have shaped contemporary therapeutic interest in psychedelics as treatments for neuropsychiatric conditions.

Recent studies have shown that classical indolethylamine psychedelics promote neuroplasticity and modulate serotonergic circuits, primarily through 5-HT_{2A} receptor activation. These compounds have demonstrated therapeutic potential for depression, anxiety, PTSD, and addiction, with psilocybin receiving FDA Breakthrough Therapy designation for major depressive disorder in 2019. Although widely considered hallucinogenic, psilocybin itself functions as a prodrug, undergoing enzymatic dephosphorylation in the digestive tract and liver to produce psilocin, the active compound responsible for its psychoactive effects. Among natural psychedelics, DMT is notable for its rapid and intense psychoactive effects. It is produced by a broad range of plant species and, in low abundance, by certain animals. The synergistic interaction between DMT and β-carbolines from ayahuasca exemplifies the therapeutic potential of compound combinations from different sources.

The resurgence in psychedelic research has accelerated the development of novel biocatalytic systems for the heterologous production of indolethylamines. Following the complete elucidation of the psilocybin biosynthetic pathway, which involves four enzymes (FIG. 7B), several microbial systems for psilocybin bioproduction have been developed (9). In toads, a novel N-methyltransferase (NMT) was recently identified that catalyzes the N-methylation of serotonin to bufotenin (FIG. 7B). However, no additional enzymes from toads have been identified to enable the complete heterologous production of 5-MeO-DMT. Interestingly, the newly identified NMT exhibited substrate promiscuity, accepting diverse indolethylamine substrates in vitro, suggesting its potential to generate different psychedelics.

Surprisingly, until now the simple biosynthetic pathway of DMT in plants, consisting of only two types of reactions, remained unresolved. DMT biosynthesis was predicted to begin with the decarboxylation of tryptophan (Trp 1) by a tryptophan decarboxylase (TDC) to form tryptamine (TAM 2), which is then sequentially N-methylated by one or more NMTs to produce N-methyltryptamine (NMT 3) and DMT 4 (FIG. 1A). Although various natural indolethylamines originate from tryptophan-derived precursors, their biosynthetic pathways have diverged independently across species to yield structurally distinct psychedelics (FIG. 7B). Notably, no study has yet reconstructed the complete biosynthetic pathways of psychedelics within a single heterologous system.

### SUMMARY

In this study, the inventors elucidated the biosynthetic pathway of DMT in plants by identifying two pairs of TDC and NMT enzymes from the plants P. viridis and Acacia acuminata (A. acuminata). The two NMTs, which are the first tryptamine NMTs identified from plants, exhibited promiscuous in vitro activity, accepting various primary indolethylamine substrates and facilitating the production of other classical psychedelics. Building on this discovery, the inventors transiently reconstructed the complete biosynthetic pathways for both natural and novel indolethylamine psychedelics in Nicotiana benthamiana by co-expressing enzymes from plants, mushrooms, animals, and bacteria. The inventors also performed modeling and rational design of a key enzyme to enhance psychedelic production. This strategy enabled the development of a scalable, sustainable plant heterologous system for producing these therapeutically valuable compounds, potentially paving the way for advanced metabolic engineering to increase yields and explore other novel psychoactive derivatives.

According to the first aspect, there is provided an artificial DNA molecule comprising a nucleic acid sequence having at least 80% identity or homology to SEQ ID NO: 1.

According to another aspect, there is provided an artificial DNA molecule comprising a nucleic acid sequence having at least 80% identity or homology to SEQ ID NO: 2.

According to another aspect, there is provided an artificial DNA molecule comprising a nucleic acid sequence having at least 80% identity or homology to SEQ ID Nos: 3 and 7.

According to another aspect, there is provided a vector comprising the artificial DNA molecule of the invention.

According to another aspect, there is provided a polypeptide encoded by any one of: (a) the artificial DNA molecule of the invention; and (b) the vector of the invention.

According to another aspect, there is provided a transgenic or transfected cell comprising: (a) the artificial DNA molecule of the invention; (b) the vector of the invention; (c) the polypeptide of the invention; or (d) any combination of (a) to (c).

According to another aspect, there is provided an extract, lysate, or homogenate derived from the transgenic or transfected cell of the invention, or any fraction thereof.

According to another aspect, there is provided a composition comprising: (a) the artificial DNA molecule of the invention; (b) the vector of the invention; (c) the polypeptide of the invention; (d) the transgenic or transfected cell of the invention; (e) the extract of the invention; or (f) any combination of (a) to (e), and an acceptable carrier.

According to another aspect, there is provided a method for synthesizing tryptamine or a metabolite thereof, the method comprising: (a) providing a cell comprising at least one first artificial DNA molecule comprising a nucleic acid sequence having at least 80% homology to any one of SEQ ID Nos: 1-2; and (b) culturing the cell from step (a) such that a first polypeptide encoded by the at least one first artificial DNA molecule is expressed, thereby synthesizing tryptamine.

According to another aspect, there is provided a method for synthesizing tryptamine comprising contacting tryptophan with an effective amount of a recombinant protein comprising an amino acid sequence having at least 84% homology or identity to any one of SEQ ID Nos: 4-5, thereby synthesizing tryptamine.

According to another aspect, there is provided a method for synthesizing NMT comprising contacting Tryptamine with an effective amount of a recombinant protein comprising an amino acid sequence having at least 69% homology or identity to any one of SEQ ID Nos: 6, 8-9, or a combination thereof, thereby synthesizing NMT.

According to another aspect, there is provided a method for synthesizing DMT comprising contacting NMT with an effective amount of a recombinant protein comprising an amino acid sequence having at least 69% homology or identity to any one of SEQ ID Nos: 6, 8-9, or a combination thereof, thereby synthesizing DMT.

According to another aspect, there is provided a method for synthesizing N-Me-Serotonin comprising contacting Serotonin with an effective amount of a recombinant protein comprising an amino acid sequence having at least 69% homology or identity to any one of SEQ ID Nos: 6, 8-9, or a combination thereof, thereby synthesizing N-Me-Serotonin.

According to another aspect, there is provided a method for synthesizing Bufotenine comprising contacting N-Me-Serotonin with an effective amount of a recombinant protein comprising an amino acid sequence having at least 69% homology or identity to any one of SEQ ID Nos: 6, 8-9, or a combination thereof, thereby synthesizing Bufotenine.

According to another aspect, there is provided a method for synthesizing 5-MeO-NMT comprising contacting 5-MeO-tryptamine with an effective amount of a recombinant protein comprising an amino acid sequence having at least 69% homology or identity to any one of SEQ ID Nos: 6, 8-9, or a combination thereof, thereby synthesizing 5-MeO-NMT.

According to another aspect, there is provided a method for synthesizing 5-MeO-DMT comprising contacting 5-MeO-NMT with an effective amount of a recombinant protein comprising an amino acid sequence having at least 69% homology or identity to any one of SEQ ID Nos: 6, 8-9, or a combination thereof, thereby synthesizing 5-MeO-DMT.

According to another aspect, there is provided a method for synthesizing Norpsilocin comprising contacting 4-OH-Tryptamine with an effective amount of a recombinant protein comprising an amino acid sequence having at least 69% homology or identity to any one of SEQ ID Nos: 6, 8-9, or a combination thereof, thereby synthesizing Norpsilocin.

According to another aspect, there is provided a method for synthesizing Psilocin comprising contacting Norpsilocin with an effective amount of a recombinant protein comprising an amino acid sequence having at least 69% homology or identity to any one of SEQ ID Nos: 6, 8-9, or a combination thereof, thereby synthesizing Psilocin.

According to another aspect, there is provided a method for synthesizing Baeocystin comprising contacting Norbaeocystin with an effective amount of a recombinant protein comprising an amino acid sequence having at least 69% homology or identity to any one of SEQ ID Nos: 6, 8-9, or a combination thereof, thereby synthesizing Baeocystin.

According to another aspect, there is provided a method for synthesizing Psilocybin comprising contacting Baeocystin with an effective amount of a recombinant protein comprising an amino acid sequence having at least 69% homology or identity to any one of SEQ ID Nos: 6, 8-9, or a combination thereof, thereby synthesizing Psilocybin.

According to another aspect, there is provided a method for synthesizing 5-MeO-tryptamine comprising contacting Serotonin with an effective amount of a recombinant protein comprising an amino acid sequence having at least 90% homology or identity to any one of SEQ ID Nos: 12 or 14, thereby synthesizing 5-MeO-tryptamine.

According to another aspect, there is provided a method for synthesizing 5-MeO-NMT comprising contacting N-Me-Serotonin with an effective amount of a recombinant protein comprising an amino acid sequence having at least 90% homology or identity to any one of SEQ ID Nos: 12 or 14, thereby synthesizing 5-MeO-NMT.

According to another aspect, there is provided a method for synthesizing 5-MeO-DMT comprising contacting Bufotenine with an effective amount of a recombinant protein comprising an amino acid sequence having at least 90% homology or identity to any one of SEQ ID Nos: 12 or 14, thereby synthesizing 5-MeO-DMT.

In some embodiments, the nucleic acid sequence encodes a protein being a decarboxylase. In some embodiments, the decarboxylase is tryptophan decarboxylase. In some embodiments, the nucleic acid sequence encodes a protein being a methyltransferase.

In some embodiments, the methyltransferase is N-methyltransferase (NMT). In some embodiments, the artificial DNA molecule is 900 to 1,600 base pairs (bp) long. In some embodiments, the vector is a plasmid or an agrobacterium. In some embodiments, the vector further comprises at least one additional nucleic acid sequence encoding at least one enzyme selected from the group consisting of: CYP71P1, O-methyltransferase (OMT), PsiH, PsiK, PsiM, and any combination thereof. In some embodiments, the polypeptide is a recombinant polypeptide. In some embodiments, the polypeptide comprises an amino acid sequence with at least 85% homology to any one of SEQ ID Nos: 4-6, and 8. In some embodiments, the polypeptide is characterized by having an activity of: (i) releasing a carboxyl group from a substrate; or (ii) transferring a methyl group from a donor molecule to a substrate. In some embodiments, the culturing comprises supplementing the cell with an effective amount of tryptophan. In some embodiments, the cell further comprises at least one second artificial DNA molecule comprising a nucleic acid sequence having at least 80% homology to any one of SEQ ID Nos: 3, 7, and 10, and step (b) comprises culturing the cell from step (a) such that a second polypeptide encoded by the at least one second artificial DNA molecule is expressed, thereby synthesizing N-methyltryptamine (NMT), N,N-dimethyltryptamine (DMT), or both. In some embodiments, the cell further comprises at least one third artificial DNA molecule comprising a nucleic acid sequence encoding at least one enzyme selected from the group consisting of: CYP71P1, 4-Hydroxylase, 5-Hydroxylase, PsiH, PsiK, OMT, or any combination thereof, and step (b) comprises culturing the cell from step (a) such that at least one third polypeptide encoded by the at least one third artificial DNA molecule is expressed, thereby synthesizing the tryptamine metabolite being selected from the group consisting of: Serotonin, 5-methoxytryptamine, 4-OH-Trytamine, Norbaeocystin, 5-methoxy-N-methyltryptamine (5-MeO-NMT), N-methyl-Serotonin (N-Me-Serotonin), Norpsilocin, Baeocystin, 5-methoxy-DMT (5-MeO-DMT), Bufotenine, Psilocin, Psilocybin, and any combination thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1C include chemical structures, photographs, and heat maps showing that N,N-dimethyl tryptamine (DMT) accumulates across different tissues of Psychotria viridis and Acacia acuminata plants. (1A) Predicted biosynthetic pathway of DMT 1 in plants. (1B) Representative photographs of Psychotria viridis, Psychotria carthagenensis and Acacia acuminata plants and tissues and heatmap of absolute concentrations of metabolites 1~4 in the different tissues (% w/w per fresh weight; mean, n=3). Non-detected metabolites appear in white. (1C) Matrix-assisted laser desorption/ionization-mass spectrometry imaging (MALDI-MSI) of Acacia acuminata stem cross section (n=2). Individual and overlay visualization of tryptamine (TAM 2, [M+H]⁺, m/z 161.109±0.01 Da), N-methyl tryptamine (NMT 3, [M+K]⁺, m/z 213.078±0.01 Da) and N,N-dimethyl tryptamine (DMT 4, [M+K]⁺, m/z 227.094±0.01 Da). Visible areas in the stem cross section are marked on the optical image. Overlay 1 and Overlay 2 correspond to overlay of molecules 2-4 with and without the optical image, respectively.

FIGS. 2A-2G include graphs, chromatograms, a scheme, and chemical structures showing the functional characterization of tryptophan decarboxylase and N-methyltransferase enzymes from Psychotria viridis. (2A) Comparison of ion abundance peak areas (mean±SD; n=3 biological replicates) of tryptamine (TAM 2) following in vitro assays using lysate from E. coli cells transformed with tryptophan decarboxylase (TDC) candidates, using tryptophan (Trp 1) as a substrate in the presence of pyridoxal 5′-phosphate (PLP). Statistical significance was assessed using a two-tailed Student's t-test with an assumption of unequal variance; **** p<0.0001. (2B) Extracted ion chromatograms of TAM 2 following in vitro assays with purified PvTDC2 and TAM 2 as a substrate in the presence of PLP (n=3 biological replicates). Chromatograms were normalized to the highest value. (2C) Comparison of ion abundance peak areas (mean±SD; n=3-4 biological replicates) following transient expression of PvTDC1 and PvTDC2 in Nicotiana benthamiana (N. benthamiana) leaves. Statistical significance was assessed using one-way analysis of variance (ANOVA) followed by Tukey's multiple comparisons as a post hoc test; letters denote statistically significant differences (p<0.05). (2D) Co-expression network showing isoforms correlated with at least one of three baits-two possible isoforms of PvTDC1 (green) and one of PvTDC2 (blue). Pairwise-Pearson correlation coefficients were calculated and a threshold of r>0.8 was set. The number of co-expressed isoforms per bait is indicated. N-methyltransferase candidates and partial methyltransferase isoforms are highlighted. (2E) Comparison of ion abundance peak areas (mean±SD; n=3 biological replicates) of NMT 3 and DMT 4 following in vitro assays using lysate from E. coli cells transformed with NMT candidates, using TAM 2 or NMT 3 as substrates (marked in blue), respectively, in the presence of S-adenosyl-L-methionine (SAM) as the methyl donor. Statistical significance was assessed using a two-tailed Student's t-test with an assumption of unequal variance; *** p<0.001, **** p<0.0001. Plus signs indicate the biosynthetic genes included in each experiment. (2F) Extracted ion chromatograms of Trp 1 (m/z 205.097), TAM 2 (m/z 161.107), N-methyl-tryptamine (NMT 3, m/z 175.123) and N,N-dimethyl-tryptamine (DMT 4, m/z 189.138) following in vivo experiments with E. coli cells co-expressing PvTDC2 and PvNMT1 (n=3 biological replicates). Cell supernatants were collected 24 h following induction of proteins. (2G) Comparison of ion abundance peak areas (mean±SD; n=4 biological replicates) following transient expression of PvTDC2 and PvNMT1 in N. benthamiana leaves. Statistical significance was assessed using one-way ANOVA followed by Tukey's multiple comparisons as a post hoc test; letters denote statistically significant differences (p<0.05). The plus signs (+) indicate the biosynthetic genes included in each experiment. All metabolites were identified by exact mass, retention time, and MS/MS spectra. EV, empty vector; nd, not detected.

FIGS. 3A-3C include chromatograms, a phylogenetic tree, and chemical structures showing the promiscuous in vitro activity of PvNMT1 and AaNMT1. (3A) Extracted ion chromatograms of N-methyl-tryptamine (NMT 3) and N,N-dimethyl-tryptamine (DMT 4) following in vitro assays with lysate from E. coli cells expressing N-methyltransferase (NMT) candidates from Acacia acuminata. Assays were performed with S-adenosyl-L-methionine (SAM) as the methyl donor, and either tryptamine (TAM 2) or NMT 3 as substrates (marked on the top). The active enzymes appear in purple. Metabolites were identified by exact mass, retention time, and MS/MS spectra. EV, empty vector. (3B) Phylogenetic analysis of methyltransferases, including newly identified N-methyltransferases (NMTs) from Psychotria viridis (highlighted in red) and Acacia acuminata (highlighted in purple). Functionally characterized O-methyltransferases (OMTs) and NMTs from other plants were compared (data not shown). Other candidate methyltransferases tested in this study are shown in orange. Functionally characterized NMTs from plants are marked in pink. Indolethylamine NMTs from the cane toad (Rhinella marina, RmNMT, shown in green) and from Psilocybe cubensis (PsiM, shown in blue) were also included in the analysis. All three identified NMTs from this study clustered with flavonoid-type OMTs (clade marked in red). The Maximum Likelihood tree was constructed with 1,000 bootstrap tests based on a MUSCLE multiple alignment using the MEGA11 software. (3C) Combined extracted ion chromatograms following in vitro assays with lysate from E. coli cells expressing PvNMT1 (marked in red) or AaNMT1 (marked in purple), with SAM and either TAM 2, serotonin (5-HT 5), 5-methoxytryptamine (5-MeO-TAM 8), 6-hydroxytryptamine (6-HT 11), 4-hydroxytryptamine (4-HT 14) or norbaeocystin 17. Samples were collected following two hours of incubation. The chemical structures of substrates are shown for reference. Metabolites in each assay were identified by comparison with analytical standards (numbers marked in blue), an extract from Psilocybe cubensis mushrooms or putatively assigned according to accurate mass, relative retention time and MS/MS spectra (see FIG. 12B). Chromatograms were normalized to the highest value per substrate. EV, empty vector.

FIG. 4 includes a a scheme showing complete reconstruction of psychedelic indolethylamine biosynthetic pathways in Nicotiana benthamiana. Biosynthetic pathways were reconstructed using genes from plants, fungi, and toads. The biosynthetic pathway leading to N,N-dimethyltryptamine (DMT 4) identified in this study is highlighted in red. Confirmed steps are indicated with bold arrows. All tested enzymes for each step are listed next to the corresponding arrow, with the most active enzymes marked in bold. Enzymes from each organism are color-coded as follows: Psychotria viridis (P. viridis) and Acacia acuminata (A. acuminata) in red; Rhinella marina (R. marina) in green; Psilocybe cubensis (P. cubensis) in blue; Oryza sativa (O. sativa) in brown; and Arabidopsis thaliana in pink. Representative images of the organisms are provided next to the corresponding products and/or enzymes. Novel reactions catalyzed by the mutated AtCOMT-Mut1 (SEQ ID Nos: 13-14) enzyme are indicated with dotted lines. Enzymes with question mark correspond to steps that were not confirmed. R¹ and R² correspond to H, OH, OCH₃, or H₂PO₄ groups of the psychedelic metabolite. Trp 1, tryptophan; TAM 2, tryptamine; NMT 3, N-methyltryptamine; 5-HT 5, serotonin; 5-HNMT 6, 5-hydroxy-N-methyltryptamine; 5-MeO-TAM 8, 5-methoxytryptamine; 5-MeO-NMT 9, 5-methoxy-N-methyltryptamine; 5-MeO-DMT 10, 5-methoxy-N,N,-dimethyltryptamine; 4-HT 14, 4-hydroxytryptamine.

FIGS. 5A-5D include graphs, chemical structures, a three-dimensional structure, and a scheme showing de novo bioproduction of psychedelic indolethylamines in Nicotiana benthamiana plants. Comparison of absolute concentrations of psychedelic products (mean±SD; n=4 biological replicates; values are given in μg·g⁻¹ per fresh weight) following transient expression of different combinations of AroG, PvTDC2, and (5A) PvNMT1, PsiH, PsiM, and PsiK, or (5B) OsCYP71P1, RmNMT, AtCOMT, the mutant AtCOMT-Mut1 (SEQ ID Nos: 13-14), and PvNMT1 in N. benthamiana leaves. Absolute concentrations were according to external calibration curves. Concentrations of bufotenine 7 and 5-methoxy-N-methyltryptamine (5-MeO-NMT 9) were semi-quantified using the calibration curves of psilocin 16 and 5-methoxy-N,N-dimethyltryptamine (5-MeO-DMT 10), respectively. Samples were collected one week following agroinfiltration. Unless otherwise stated, statistical significance was assessed using one-way ANOVA followed by Tukey's post hoc test; different letters denote statistically significant differences (p<0.05; ns, not significant). Statistical significance of psilocin 16, 5-MeO-NMT 9 and 5-MeO-DMT 10 was assessed using a two-tailed Student's t-test assuming unequal variance (** p<0.01; *** p<0.001; **** p<0.0001). Chemical structures of products are shown for reference and are colored according to FIG. 4. Additional pathway intermediates are shown in FIGS. 15-16. The plus signs (+) indicate the biosynthetic genes included in each experiment; boxes mark the last enzyme added in a set of co-expressed genes. (5C) AlphaFold model of AtCOMT with S-adenosyl-L-methionine (SAM) cofactor and bufotenine 7 as the substrate. Inset: clash between A160 (magenta) and bufotenin 7 methyls (purple), shown as spheres. Sequence alignment of AtCOMT and AtCOMT-Mut1 (SEQ ID NO: 14) is shown for reference, with the mutated residue highlighted in magenta. (5D) Absolute concentrations of psychedelic products (mean±SD; n=6 biological replicates; values are given in μg·g⁻¹ per fresh weight) following transient expression of nine genes in N. benthamiana leaves. Statistical significance was assessed using a two-tailed Student's t-test assuming unequal variance (*p<0.5; ** p<0.01; *** p<0.001). The co-expressed genes appear on the bottom and are colored according to FIG. 4. Samples were collected one week following infiltration. Products were identified by comparison with analytical standards, extracts from Psilocybe cubensis mushrooms, or putatively assigned based on accurate mass, relative retention time, and MS/MS spectra, as detailed previously (FIG. 12B). DMT 4, N,N-dimethyltryptamine; EV, empty vector; nd, not detected.

FIGS. 6A-6B include chemical structures, tables, and chromatograms showing de novo bioproduction of halogenated indolethylamines in N. benthamiana. Extracted ion chromatograms of (6A) chlorinated and brominated N,N-dimethyltryptamine (molecules 38-40 and 41-43, respectively) and (6B) chlorinated psilocybin (molecules 48 and 49) following agroinfiltration of different combinations of genes as presented in the figure (n=4 biological replicates). Samples were collected five days following agroinfiltration. RebH, PyrH and SttH halogenated the carbons in positions 7, 5 and 6, respectively (annotated carbons on DMT 4 and psilocybin 19 chemical structures are marked in red, blue and green, respectively). Comparison of peak areas for all the halogenated intermediates from the pathways appear in FIGS. 20 and 22. Peaks were identified based on accurate mass, relative retention time, and MS/MS spectra and putatively assigned to the corresponding halogenated products according to previous reports (full details appear in FIGS. 20-22). Chromatograms were normalized to the highest value. EV, empty vector.

FIGS. 7A-7B include chemical structures, illustrations, and a flow showing the divergent evolution of biosynthetic pathways for natural indolethylamine psychedelics with therapeutic potential across eukaryotic life. (7A) Simplified phylogenetic relationships among genera within the Eukaryota kingdom and the corresponding chemical structures of major psychedelics accumulated by representative species. N,N-dimethyltryptamine (DMT) (plants), psilocybin (magic mushrooms), and bufotenin and 5-methoxy-N,N-dimethyltryptamine (5-MeO-DMT) (toads) are highlighted in pink, blue, and green, respectively. Functionally characterized biosynthetic enzymes elucidated from each species are listed alongside representative images. (7B) Predicted relationships between indolethylamine biosynthetic pathways illustrate divergent evolutionary origins across the Eukaryota kingdom. The biosynthetic pathway of Psilocybe cubensis (P. cubensis) magic mushrooms has been fully elucidated; other pathways remain predicted. Serotonin biosynthesis can proceed either via 5-hydroxylation of tryptophan followed by decarboxylation (typical of mammals), or via decarboxylation to tryptamine followed by 5-hydroxylation (observed in plants). Functionally characterized enzymes from P. cubensis and the cane toad are shown in blue and green, respectively. The chemical structure of tryptamine is shown with labeled 4- and 5-positions to indicate the sites of hydroxylation.

FIGS. 8A-8B includes heatmaps and photographs showing the metabolic screening of plant tissues reported to accumulate N,N-dimethyltryptamine. (8A) High-resolution liquid chromatography-mass spectrometry (LC-MS) peak areas (z-scores) of N-methyltryptamine (NMT 3) and N,N-dimethyltryptamine (DMT 4) across different tissues. Metabolites were identified using authentic analytical standards. (8B) Representative photographs of selected sampled plant species.

FIGS. 9A-9D include chemical structures and chromatograms showing the identification of major indolethylamines in Psychotria viridis and Acacia acuminata plants by analytical standards. (9A-9D) MS/MS spectral matching of tryptophan (Trp 1), tryptamine (TAM 2), N-methyltryptamine (NMT 3), and N,N-dimethyltryptamine (DMT 4) standards versus Psychotria viridis (P. viridis) leaves and Acacia acuminata (A. acuminata) stem samples, respectively. The metabolites were identified according to retention time, accurate mass and MS/MS spectra.

FIGS. 10A-10E include a photograph, a heat map, and chromatograms showing functional characterization of Psychotria viridis enzymes. (10A) SDS-PAGE gel electrophoresis of purified PvTDC1 and PvTDC2 proteins. Bands corresponding to the purified enzymes are marked with arrows. The faint band for PvNMT1 suggests poor in vitro expression or protein instability. EV, empty vector. (10B) Gene expression heatmap (z-score of mean, n=3) of the characterized enzymes across P. viridis tissues. (10C) Extracted ion chromatograms from in vitro enzyme assays using E. coli lysate expressing either PvNMT1 or PvNMT2, with S-adenosyl-L-methionine (SAM) as the methyl donor and either tryptamine (TAM 2) or N-methyltryptamine (NMT 3) as substrates (noted above each trace). Products were identified using analytical standards. (10D) Extracted ion chromatograms of tryptophan (Trp 1), TAM 2, NMT 3, and N,N-dimethyl-tryptamine (DMT 4) from in vitro assays using mixtures of E. coli lysate expressing PvNMT1 and either PvTDC1 or PvTDC2 (n=3 biological replicates). Assays included pyridoxal 5′-phosphate (PLP), SAM and Trp 1 as the substrate. Only the combination of PvTDC2 and PvNMT1 yielded metabolites 2-4. (10E) MS/MS spectral matching of metabolites 1-4 with analytical standards from in vivo assays using E. coli co-expressing PvTDC2 and PvNMT1 (n=3 biological replicates). Supernatants were collected 24 h post-induction.

FIGS. 11A-11C include a multiple amino acid sequence alignment of active enzymes from Psychotria viridis and their closest homologs in Psychotria carthagenensis. Amino acid alignments of (11A) PvTDC1, (11B) PvTDC2, and (11C) PvNMT1 with their respective homologous isoforms from P. carthagenensis. Residue differences in P. carthagenensis compared to the corresponding P. viridis enzymes are highlighted.

FIGS. 12A-12B include chemical structures and chromatograms showing in vitro reactions with PvNMT1 and AaNMT1 and putative identification of products. (12A) In vitro reactions catalyzed by lysate expressing PvNMT1 or AaNMT1 with S-adenosyl-L-methionine (SAM) as the methyl donor, and either tryptamine (TAM 2), serotonin (5-HT 5), 5-methoxytryptamine (5-MeO-TAM 8), 6-hydroxytryptamine (6-HT 11), or 4-hydroxytryptamine (4-HT 14) as the substrates. Neither PvNMT1 nor AaNMT1 showed activity with norbaeocystin 17. Samples were collected following two hours of incubation. TAM 2, tryptamine; NMT 3, N-methyltryptamine; DMT 4, N,N-dimethyltryptamine; 5-HNMT 6, 5-hydroxy-N-methyltryptamine; 5-MeO-NMT 9, 5-methoxy-N-methyltryptamine; 5-MeO-DMT 10, 5-methoxy-N,N,-dimethyltryptamine; 6-HNMT 12, 6-hydroxy-N-methyltryptamine; 6-HDMT 13, 6-hydroxy-N,N-dimethyltryptamine. (12B) Putative identification of indolethylamine products based on MS/MS fragmentation spectra. Metabolites were identified by fragmentation patterns similar to the standard metabolites (highlighted in red), with mass shifts consistent with the addition of one or two methyl groups, and retention time increases corresponding to the degree of methylation. Characteristic fragment ions corresponding to N-methyl (C₂H₆N) and N,N-dimethyl (C₃H₈N) groups were detected in most cases. Substrates 5-, 6-, and 4-HT (metabolites 5, 11, and 14) and their respective N-methylated and N,N-dimethylated products showed similar fragmentation patterns. Metabolites 14-16 displayed fragmentation profiles comparable to peaks detected in Psilocybe cubensis extracts.

FIGS. 13A-13C include chemical structures, graphs, and chromatograms showing the de novo bioproduction of primary indolethylamines in Nicotiana benthamiana leaves. (13A) Ion abundance peak areas of primary indolethylamines (mean±SD; n=3-4 biological replicates) following transient expression of different combinations of PvTDC2, OsCYP71P1, PsiH, and PsiK in N. benthamiana leaves. Statistical significance of tryptophan (Trp 1) and tryptamine (TAM 2) was assessed by one-way ANOVA followed by Tukey's post hoc test; different letters indicate statistically significant differences (p<0.05). Statistical significance of 4-hydroxytryptamine (4-HT 14) was assessed using a two-tailed Student's t-test with an assumption of unequal variance (** p<0.01). Chemical structures of the products are shown for reference. The plus signs (+) indicate biosynthetic genes included in each experiment; boxes mark the last enzyme added in a set of co-expressed genes. Chemical structures of products are shown for reference and are colored according to the pathways in FIG. 4. Metabolites were identified using analytical standards based on accurate mass, relative retention time, and MS/MS spectra. EV, empty vector; nd, not detected; 5-HT 5, serotonin. (13B) Extracted ion chromatograms and MS/MS spectral matching of metabolites 5, 14, and 17 following agroinfiltration to N. benthamiana leaves with the indicated enzyme combinations. Identification of metabolites was according to analytical standards. Chromatograms were normalized to the highest value. EV, empty vector. (13C) Extracted ion chromatograms of 5-methoxytryptamine (5-MeO-TAM 8) following agroinfiltration of PvTDC2, OsCYP71P1, and AtCOMT in N. benthamiana. Identification of 5-MeO-TAM 8 was based on accurate mass and retention time; MS/MS spectra could not be acquired for this peak due to low abundance. Samples were collected four days following infiltration.

FIGS. 14A-14C include graphs showing N-Methyltransferase activities for bioproduction of tertiary indolethylamines, i.e., Trp 1, TAM 2, NMT 3, and DMT 4 (14A), 5-HT 5,5-HNMT 6, Bufotenin 7, and 5-MeO-DMT 10 (14B), and 4-OH-TAM 14, Norpsilocin 15, Psilocin 16, Norbaeocystin 17, and Baeocystin 18 (14C) in Nicotiana benthamiana leaves. Ion abundance peak areas of indolethylamines (mean±SD; n=3-4 biological replicates) following transient expression of different combinations of enzymes in N. benthamiana leaves. Unless otherwise stated, statistical significance was assessed using one-way ANOVA followed by Tukey's post hoc test; different letters indicate statistically significant differences (p<0.05). For 5-methoxy-N,N-dimethyltryptamine (5-MeO-DMT 10) and psilocin 16, statistical significance was assessed using a two-tailed Student's t-test assuming unequal variance (ns, not significant). The plus signs (+) indicate biosynthetic genes included in each experiment; boxes highlight the N-methyltransferase enzyme added to each gene combination (red: PvNMT1, blue: AaNMT1, green: RmNMT). Metabolites were identified by comparison with analytical standards, extracts from Psilocybe cubensis mushrooms, or putatively assigned based on accurate mass, relative retention time, and MS/MS spectra, as detailed in FIG. 12B. Samples were collected five days following infiltration. EV, empty vector; nd, not detected; ns, not significant; Trp 1, tryptophan; TAM 2, tryptamine; NMT 3, N-methyltryptamine; DMT 4, N,N-dimethyltryptamine; 5-HT 5, serotonin; 5-HNMT 6, 5-hydroxy-N-methyltryptamine; 5-MeO-TAM 8, 5-methoxytryptamine; 5-MeO-NMT 9, 5-methoxy-N-methyltryptamine; 4-HT 14, 4-hydroxytryptamine.

FIGS. 15A-15D include graphs showing the de novo bioproduction of primary and secondary indolethylamines from the DMT and psilocybin pathways in Nicotiana benthamiana plants. (15A) Comparison of ion abundance peak areas of tryptophan (Trp 1), tyrosine and phenylalanine (mean±SD; n=4 biological replicates) following transient expression of AROG in N. benthamiana leaves. Statistical significance was assessed using a two-tailed Student's t-test assuming unequal variance (** p<0.01; *** p<0.001). EV, empty vector. (15B) Comparison of ion abundance peak areas of primary and secondary indolethylamines (mean±SD; n=4 biological replicates) following transient expression of different combinations of AROG, PvTDC2, PVNMTI, PsiH, PsiM, and PsiK in N. benthamiana leaves. Statistical significance was assessed using one-way ANOVA followed by Tukey's post hoc test; different letters denote statistically significant differences (p<0.05). Statistical significance of 4-hydroxytryptamine (4-HT 14) was assessed using a two-tailed Student's t-test with an assumption of unequal variance (** p<0.01). The plus signs (+) indicate biosynthetic genes included in each experiment; boxes mark the last enzyme added in a set of co-expressed genes. Products were identified by comparison with analytical standards, extracts from Psilocybe cubensis mushrooms, or putatively assigned based on accurate mass, relative retention time, and MS/MS spectra, as detailed in FIG. 12B. TAM 2, tryptamine; NMT 3, N-methyltryptamine. Comparison of absolute concentrations of (15C) N,N-dimethyltryptamine (DMT 4) and (15D) psilocin 16 and psilocybin 19 (mean±SD; n=4 biological replicates) following transient expression of DMT or psilocybin genes, respectively, with or without AroG in N. benthamiana leaves. Statistical significance was assessed using a two-tailed Student's t-test assuming unequal variance (ns, not significant; ** p<0.01; *** p<0.001). The agroinfiltrated genes in each experiment appear on the bottom. Samples were collected one week following infiltration.

FIG. 16 includes the identification of indolethylamines from the DMT and psilocybin pathways in Nicotiana benthamiana plants. Combined extracted ion chromatograms and MS/MS spectral matching of metabolites 2-4 and 14-19 following agroinfiltration to N. benthamiana leaves with the indicated enzyme combinations. Metabolites 2-4, 14 and 19 were identified by comparison with analytical standards; metabolites 15 and 16 were produced in in vitro assays with lysate expressing AaNMT1 and 4-hydroxytryptamine (4-HT 14) as the substrate (see FIG. 12B); and metabolites 14-19 were identified in an extract from Psilocybe cubensis mushrooms. Chromatograms were normalized to the highest value. The agroinfiltrated genes in each experiment appear next to the respective chromatogram. TAM 2, tryptamine; NMT 3, N-methyltryptamine; DMT 4, N,N-dimethyltryptamine; EV, empty vector.

FIG. 17 includes chemical structures and graphs showing the de novo bioproduction of primary and secondary indolethylamines from the serotonin pathway in Nicotiana benthamiana plants. Comparison of ion abundance peak areas of primary and secondary indolethylamines (mean±SD; n=4 biological replicates) following transient expression of different combinations of AroG, PvTDC2, OsCYP71P1, RmNMT, AtCOMT, the mutant AtCOMT-Mut1 (SEQ ID Nos: 13-14), and PvNMT1, in N. benthamiana leaves. Statistical significance was assessed using one-way ANOVA followed by Tukey's post hoc test; different letters denote statistically significant differences (p<0.05). Chemical structures of the products are shown for reference and are colored according to FIG. 4. The plus signs (+) indicate biosynthetic genes included in each experiment; boxes mark the last enzyme added in a set of co-expressed genes. Products were identified by comparison with analytical standards or putatively assigned based on accurate mass, relative retention time, and MS/MS spectra, as detailed in FIG. 12B. 5-HT 5, serotonin; 5-HNMT 6, 5-hydroxy-N-methyltryptamine; EV, empty vector; nd, not detected. Samples were collected one week following infiltration.

FIG. 18 includes chromatograms showing the identification of indolethylamines from the 5-MeO-DMT pathway in Nicotiana benthamiana plants. Combined extracted ion chromatograms and MS/MS spectral matching of metabolites 5-7 and 9-10 following agroinfiltration to N. benthamiana leaves with the indicated enzyme combinations. Metabolites 5 and 10 were identified by comparison with analytical standards; metabolites 6, 7, 9 and 10 were produced in in vitro assays with lysate expressing PvNMT1 and either serotonin (5-HT 5) or 5-methoxytryptamine (5-MeO-TAM 8) as the substrates (see FIG. 12B). Chromatograms were normalized to the highest value. 5-HNMT 6, 5-hydroxy-N-methyltryptamine; 5-MeO-TAM 8, 5-methoxytryptamine; 5-MeO-NMT 9, 5-methoxy-N-methyltryptamine; 5-MeO-DMT 10, 5-methoxy-N,N-dimethyltryptamine; EV, empty vector.

FIG. 19 includes a scheme of graphs showing the simultaneous production of psychedelics from different organisms in Nicotiana benthamiana plants. Comparison of ion abundance peak areas of primary and secondary indolethylamines (mean±SD; n=6 biological replicates) following transient expression of nine genes in N. benthamiana leaves. Statistical significance was assessed using a two-tailed student's t-test assuming unequal variance (*p<0.5; ** p<0.01; *** p<0.001; **** p<0.0001). Graphs are arranged according to the parallel biosynthetic pathways, with co-expressed genes and psychedelic product graphs colored according to FIG. 4. Samples were collected one week following infiltration. Novel reactions catalyzed by the mutated AtCOMT-Mut1 (SEQ ID NO: 14) enzyme are indicated with dotted lines. Enzymes with question mark correspond to steps that were not confirmed. Products were identified by comparison with analytical standards, extracts from Psilocybe cubensis mushrooms, or putatively assigned based on accurate mass, relative retention time, and MS/MS spectra, as detailed previously (FIG. 12B). Trp 1, tryptophan; TAM 2, tryptamine; NMT 3, N-methyltryptamine; DMT 4, N,N-dimethyltryptamine; 5-HT 5, serotonin; 5-HNMT 6, 5-hydroxy-N-methyltryptamine; 5-MeO-TAM 8, 5-methoxytryptamine; 5-MeO-NMT 9, 5-methoxy-N-methyltryptamine; 5-MeO-DMT 10, 5-methoxy-N,N,-dimethyltryptamine; 4-HT 14, 4-hydroxytryptamine; EV, empty vector; nd, not detected.

FIGS. 20A-20D include chemical structures and chromatograms showing the putative identification of halogenated derivatives from the DMT pathway based on MS/MS fragmentation spectra. Chlorinated and brominated metabolites of (20A) tryptophan (Trp 1), (20B) tryptamine (TAM 2), (20C) N-methyltryptamine (NMT 3), and (20D) N,N-dimethyltryptamine (DMT 4) following agroinfiltration of Nicotiana benthamiana leaves with PvTDC2, PvNMT1, RebF, and either RebH, PyrH, or SttH. Metabolites 20-43 were identified based on MS/MS fragmentation patterns similar to those of the corresponding analytical standards (shown in black), with mass shifts consistent with the addition of chlorine or bromine, and relative retention times indicative of the halogenation position. Peaks were assigned to the corresponding halogenated products according to the reported activities of RebH, PyrH, and SttH on the indole ring at positions 7, 5, and 6, respectively. Halogenated derivatives at different positions exhibited similar fragmentation patterns. Annotated positions 7, 5, and 6 on the chemical structures of compounds 1~4 are marked in red, blue, and green, respectively.

FIG. 21 includes graphs showing in planta production of halogenated derivatives from the DMT pathway in Nicotiana benthamiana leaves. Ion abundance peak areas of chlorinated and brominated indolethylamines (mean±SD; n=4 biological replicates) following transient expression of different enzyme combinations in N. benthamiana leaves. Unless otherwise stated, statistical significance was assessed using one-way ANOVA followed by Tukey's post hoc test; different letters indicate statistically significant differences (p<0.05). For molecules 22, 27, 28, 30-37, 39, 40, 42 and 43, statistical significance was assessed using a two-tailed Student's t-test assuming unequal variance (ns, not significant; * p<0.05; ** p<0.01). The plus signs (+) indicate biosynthetic genes included in each experiment; boxes highlight the halogenase enzyme added in each gene combination (red: RebH, blue: PyrH, green: SttH). Peaks were identified based on accurate mass, relative retention time, and MS/MS spectra, and were putatively assigned to the corresponding halogenated products according to the reported activities of RebH, PyrH, and SttH on the indole ring at positions 7, 5, and 6, respectively. Chemical structures of the halogenated indolethylamines appear for reference. Full spectral details appear in FIG. 20. EV, empty vector; nd, not detected; Trp 1, tryptophan; TAM 2, tryptamine; NMT 3, N-methyltryptamine; DMT 4, N,N-dimethyltryptamine.

FIGS. 22A-22B include graphs and chromatograms showing in planta production of halogenated derivatives from the psilocybin pathway in Nicotiana benthamiana leaves. (22A) Ion abundance peak areas (mean±SD; n=4 biological replicates) and (22B) MS/MS spectra of chlorinated indolethylamines following transient expression of AroG, PvTDC2, PsiHI, PsiM, PsiK, RebF, and either RebH, PyrH, or SttH in N. benthamiana leaves. Unless otherwise stated, statistical significance was assessed using one-way ANOVA followed by Tukey's post hoc test; different letters indicate statistically significant differences (p<0.05). For 7-Cl-psilocybin 48, statistical significance was assessed using a two-tailed Student's t-test assuming unequal variance (** p<0.01). The plus signs (+) indicate biosynthetic genes included in each experiment; boxes highlight the halogenase enzyme added in each gene combination (red: RebH, blue: PyrH, green: SttH). Metabolites 44-49 were identified based on MS/MS fragmentation patterns similar to those of the corresponding non-halogenated standards (shown in black), with mass shifts consistent with chlorine addition, and relative retention times indicative of the halogenation position. Compounds were assigned to the corresponding halogenated products based on the reported activities of RebH, PyrH, and SttH, which halogenate the indole ring at positions 7, 5, and 6, respectively. Halogenated derivatives at different positions exhibited similar fragmentation patterns. Phosphorylated metabolites were not halogenated at the 5-position.

FIG. 23 includes graphs showing bioactivity testing of AtCOMT mutants via transient expression in Nicotiana benthamiana. Ion abundance peak areas of indolethylamine intermediates from the 5-methoxy-N,N-dimethyltryptamine (5-MeO-DMT 10) pathway were compared following transient expression of AroG, PvTDC2, OsCYP71P1, RmNMT, and either wild-type AtCOMT or one of its mutants (AtCOMT-Mut1 (SEQ ID Nos: 13-14), Mut3-Mut8) in N. benthamiana leaves. The specific amino acid substitutions for each mutant are listed under SEQ ID Nos: 14-20. Leaf samples were collected one week following agroinfiltration. The plus signs (+) indicate the biosynthetic genes used in each expression combination. Mean peak area ratios of 5-MeO-DMT 10 produced by each mutant relative to wild-type AtCOMT are shown.

FIGS. 24A-24C include photographs, chemical structures, graphs, and chromatograms showing in vitro evaluation of AtCOMT and AtCOMTA160G enzymes. (24A) SDS-PAGE analysis of lysate and purified fractions expressing AtCOMT or AtCOMTA160G proteins. Bands corresponding to the monomeric and dimeric forms are indicated by red and yellow arrows, respectively. In the purified fraction, AtCOMTA160G bands appeared at substantially lower abundance compared to wild-type AtCOMT, whereas in the lysate both enzymes showed comparable expression levels. Comparison of (24B) ion abundance peak areas (median±SD; n=6-7 biological replicates from three independent rounds of culture growth and lysis) and (24C) extracted ion chromatograms of 5-methoxytryptamine (5-MeO-TAM 8), 5-methoxy-N-methyltryptamine (5-MeO-NMT 9), and 5-methoxy-N,N-dimethyltryptamine (5-MeO-DMT 10) following assays using lysate from E. coli cells transformed with AtCOMT or AtCOMTA160G, using serotonin (5-HT 5), 5-hydroxy-N-methyltryptamine (5-HNMT 6) or bufotenin 7 as substrates, respectively, in the presence of S-adenosyl-L-methionine (SAM) as the methyl donor. An extract from N. benthamiana leaves co-expressing AroG, PvTDC2, OsCYP71P1 and RmNMT served as the bufotenin 7 substrate in the enzyme assays. Statistical significance was assessed using a two-tailed Student's t-test with an assumption of unequal variance (*p<0.05; ** p<0.01; **** p<0.0001). Chromatograms were normalized to the highest value. EV, empty vector.

### DETAILED DESCRIPTION

### Methods of Synthesis

According to the first aspect, there is provided a method for synthesizing tryptamine or a metabolite thereof.

In some embodiments, the method comprises (the steps of): (a) providing a cell comprising at least one first artificial DNA molecule comprising a nucleic acid sequence having at least 80% homology to any one of SEQ ID Nos: 1-2; and (b) culturing the cell from step (a) such that a first polypeptide encoded by the at least one first artificial DNA molecule is expressed.

In some embodiments, the method comprises culturing a cell comprising at least one first artificial DNA molecule comprising a nucleic acid sequence having at least 80% homology to any one of SEQ ID Nos: 1-2; such that a first polypeptide encoded by the at least one first artificial DNA molecule is expressed.

In some embodiments, the culturing comprises supplementing the cell with an effective amount of tryptophan.

In some embodiments, the cell further comprises at least one second artificial DNA molecule comprising a nucleic acid sequence having at least 80% homology to any one of SEQ ID Nos: 3 and 7.

In some embodiments, (step (b) of) the method comprises culturing the cell (from step (a)) such that a second polypeptide encoded by the at least one second artificial DNA molecule is expressed, thereby synthesizing N-methyltryptamine (NMT), N,N-dimethyltryptamine (DMT), or both.

In some embodiments, the cell further comprises at least one third artificial DNA molecule. In some embodiments, the at least one third artificial DNA molecule comprises a nucleic acid sequence encoding at least one enzyme selected from: CYP71P1, 4-Hydroxylase, 5-Hydroxylase, PsiH, PsiK, PsiM, OMT, or any combination thereof.

In some embodiments, OMT is caffeic acid OMT (COMT). In some embodiments, COMT comprises a nucleic acid sequence derived from Arabidopsis thaliana. In some embodiments, CYP71P1 comprises a nucleic acid sequence derived from a rice species belonging to the genus Oryza. In some embodiments, a rice species belonging to the genus Oryza is or comprises Oryza sativa. In some embodiments, CYP71P1 comprises or is OsCYP71P1. In some embodiments, any one of PsiH, PsiK, PsiM, comprises a nucleic acid sequence derived from a fungus, In some embodiments, the fungus comprises or is Psilocybe cubensis.

In some embodiments, PsiH comprises a nucleic acid sequence as set forth in the GenBank (NCBI) under accession number MF000993.1, or an analogue thereof having at least 80% homology or identity thereto.

In some embodiments, PsiK comprises a nucleic acid sequence as set forth in the GenBank (NCBI) under accession number KY984099, or an analogue thereof having at least 80% homology or identity thereto.

In some embodiments, PsiM comprises a nucleic acid sequence as set forth in the GenBank (NCBI) under accession number KY984100.1, or an analogue thereof having at least 80% homology or identity thereto.

In some embodiments, CYP7191 comprises a nucleic acid sequence as set forth in the GenBank (NCBI) under accession number AK071599, or an analogue thereof having at least 80% homology or identity thereto.

In some embodiments, OMT comprises a nucleic acid sequence as set forth in the GenBank (NCBI) under accession number At5g54160, or an analogue thereof having at least 80% homology or identity thereto.

In some embodiments, (step (b) of) the method comprises culturing the cell (from step (a)) such that at least one third polypeptide encoded by the at least one third artificial DNA molecule is expressed. In some embodiments, a tryptamine metabolite is selected from: NMT, DMT, Serotonin, 5-methoxytryptamine, 4-OH-Trytamine, Norbaeocystin, 5-methoxy-N-methyltryptamine (5-MeO-NMT), N-methyl-Serotonin (N-Me-Serotonin), Norpsilocin, Baeocystin, 5-methoxy-DMT (5-MeO-DMT), Bufotenine, Psilocin, Psilocybin, or any combination thereof.

In some embodiments, the method comprises (the steps of): (a) providing a cell comprising an artificial DNA molecule comprising SEQ ID NO: 1 or 2; or a nucleic acid sequence having at least 80% homology thereto, and (b) culturing the cell from step (a) such that a polypeptide encoded by the artificial DNA molecule is expressed, thereby synthesizing tryptamine.

In some embodiments, the method comprises (the steps of): (a) providing a cell comprising an artificial DNA molecule comprising: (i) SEQ ID NO: 1 or 2; and (ii) SEQ ID NO: 3, 7, or 10; or a nucleic acid sequence having at least 80% homology thereto, and (b) culturing the cell from step (a) such that polypeptides encoded by the artificial DNA molecule are expressed, thereby synthesizing NMT.

In some embodiments, the method comprises (the steps of): (a) providing a cell comprising an artificial DNA molecule comprising: (i) SEQ ID NO: 1 or 2; and (ii) SEQ ID NO: 3, 7, or 10; or a nucleic acid sequence having at least 80% homology thereto, and (b) culturing the cell from step (a) such that polypeptides encoded by the artificial DNA molecule are expressed, thereby synthesizing DMT. In some embodiments, the method comprises (the steps of): (a) providing a cell comprising an artificial DNA molecule comprising: (i) SEQ ID NO: 1 or 2; and (ii) SEQ ID NO: 3 or 7; or a nucleic acid sequence having at least 80% homology thereto, and (b) culturing the cell from step (a) such that polypeptides encoded by the artificial DNA molecule are expressed, thereby synthesizing DMT.

In some embodiments, the method comprises (the steps of): (a) providing a cell comprising an artificial DNA molecule comprising: (i) SEQ ID NO: 1 or 2; and (ii) a nucleic acid sequence encoding OsCYP71P1; or a nucleic acid sequence having at least 80% homology thereto, and (b) culturing the cell from step (a) such that polypeptides encoded by the artificial DNA molecule are expressed, thereby synthesizing serotonin (5-HT).

In some embodiments, the method comprises (the steps of): (a) providing a cell comprising an artificial DNA molecule comprising: (i) SEQ ID NO: 1 or 2; (ii) a nucleic acid sequence encoding OsCYP71P1; and (iii) SEQ ID NO: 10, 3, or 7; or a nucleic acid sequence having at least 80% homology thereto, and (b) culturing the cell from step (a) such that polypeptides encoded by the artificial DNA molecule are expressed, thereby synthesizing hydroxy-N-methyltryptamine (5-HNMT). In some embodiments, the method comprises (the steps of): (a) providing a cell comprising an artificial DNA molecule comprising: (i) SEQ ID NO: 1 or 2; (ii) a nucleic acid sequence encoding OsCYP71P1; and (iii) SEQ ID NO: 10; or a nucleic acid sequence having at least 80% homology thereto, and (b) culturing the cell from step (a) such that polypeptides encoded by the artificial DNA molecule are expressed, thereby synthesizing hydroxy-N-methyltryptamine (5-HNMT).

In some embodiments, the method comprises (the steps of): (a) providing a cell comprising an artificial DNA molecule comprising: (i) SEQ ID NO: 1 or 2; (ii) a nucleic acid sequence encoding OsCYP71P1; and (iii) SEQ ID NO: 10, 3, or 7; or a nucleic acid sequence having at least 80% homology thereto, and (b) culturing the cell from step (a) such that polypeptides encoded by the artificial DNA molecule are expressed, thereby synthesizing Bufotenin. In some embodiments, the method comprises (the steps of): (a) providing a cell comprising an artificial DNA molecule comprising: (i) SEQ ID NO: 1 or 2; (ii) a nucleic acid sequence encoding OsCYP71P1; and (iii) SEQ ID NO: 10; or a nucleic acid sequence having at least 80% homology thereto, and (b) culturing the cell from step (a) such that polypeptides encoded by the artificial DNA molecule are expressed, thereby synthesizing Bufotenin. In some embodiments, the method comprises (the steps of): (a) providing a cell comprising an artificial DNA molecule comprising: (i) SEQ ID NO: 1 or 2; (ii) SEQ ID NO: 10, 3, or 7; and (iii) a nucleic acid sequence encoding OsCYP71P1; or a nucleic acid sequence having at least 80% homology thereto, and (b) culturing the cell from step (a) such that polypeptides encoded by the artificial DNA molecule are expressed, thereby synthesizing Bufotenin. In some embodiments, the method comprises (the steps of): (a) providing a cell comprising an artificial DNA molecule comprising: (i) SEQ ID NO: 1 or 2; (ii) SEQ ID NO: 3 or 7; and (iii) a nucleic acid sequence encoding OsCYP71P1; or a nucleic acid sequence having at least 80% homology thereto, and (b) culturing the cell from step (a) such that polypeptides encoded by the artificial DNA molecule are expressed, thereby synthesizing Bufotenin.

In some embodiments, the method comprises (the steps of): (a) providing a cell comprising an artificial DNA molecule comprising: (i) SEQ ID NO: 1 or 2; (ii) a nucleic acid sequence encoding OsCYP71P1; and (iii) SEQ ID NO: 11; or a nucleic acid sequence having at least 80% homology thereto, and (b) culturing the cell from step (a) such that polypeptides encoded by the artificial DNA molecule are expressed, thereby synthesizing 5-MeO-TAM.

In some embodiments, the method comprises (the steps of): (a) providing a cell comprising an artificial DNA molecule comprising: (i) SEQ ID NO: 1 or 2; (ii) a nucleic acid sequence encoding OsCYP71P1; (iii) SEQ ID NO: 11; and (iv) SEQ ID NO: 3, 7, or 10; or a nucleic acid sequence having at least 80% homology thereto, and (b) culturing the cell from step (a) such that polypeptides encoded by the artificial DNA molecule are expressed, thereby synthesizing 5-MeO-NMT.

In some embodiments, the method comprises (the steps of): (a) providing a cell comprising an artificial DNA molecule comprising: (i) SEQ ID NO: 1 or 2; (ii) a nucleic acid sequence encoding OsCYP71P1; (iii) SEQ ID NO: 11; and (iv) SEQ ID NO: 10, 3, or 7; or a nucleic acid sequence having at least 80% homology thereto, and (b) culturing the cell from step (a) such that polypeptides encoded by the artificial DNA molecule are expressed, thereby synthesizing 5-MeO-DMT. In some embodiments, the method comprises (the steps of): (a) providing a cell comprising an artificial DNA molecule comprising: (i) SEQ ID NO: 1 or 2; (ii) a nucleic acid sequence encoding OsCYP71P1; (iii) SEQ ID NO: 11; and (iv) SEQ ID NO: 10; or a nucleic acid sequence having at least 80% homology thereto, and (b) culturing the cell from step (a) such that polypeptides encoded by the artificial DNA molecule are expressed, thereby synthesizing 5-MeO-DMT.

In some embodiments, the method comprises (the steps of): (a) providing a cell comprising an artificial DNA molecule comprising: (i) SEQ ID NO: 1 or 2; (ii) a nucleic acid sequence encoding OsCYP71P1; (iii) SEQ ID NO: 10, 3, or 7; and (iv) SEQ ID NO: 13; or a nucleic acid sequence having at least 80% homology thereto, and (b) culturing the cell from step (a) such that polypeptides encoded by the artificial DNA molecule are expressed, thereby synthesizing 5-MeO-NMT. In some embodiments, the method comprises (the steps of): (a) providing a cell comprising an artificial DNA molecule comprising: (i) SEQ ID NO: 1 or 2; (ii) a nucleic acid sequence encoding OsCYP71P1; (iii) SEQ ID NO: 10, and (iv) SEQ ID NO: 13; or a nucleic acid sequence having at least 80% homology thereto, and (b) culturing the cell from step (a) such that polypeptides encoded by the artificial DNA molecule are expressed, thereby synthesizing 5-MeO-NMT.

In some embodiments, the method comprises (the steps of): (a) providing a cell comprising an artificial DNA molecule comprising: (i) SEQ ID NO: 1 or 2; (ii) a nucleic acid sequence encoding OsCYP71P1; (iii) SEQ ID NO: 10, 3, or 7; and (iv) SEQ ID NO: 11 or 13; or a nucleic acid sequence having at least 80% homology thereto, and (b) culturing the cell from step (a) such that polypeptides encoded by the artificial DNA molecule are expressed, thereby synthesizing 5-MeO-DMT. In some embodiments, the method comprises (the steps of): (a) providing a cell comprising an artificial DNA molecule comprising: (i) SEQ ID NO: 1 or 2; (ii) a nucleic acid sequence encoding OsCYP71P1; (iii) SEQ ID NO: 10; and (iv) SEQ ID NO: 11 or 13; or a nucleic acid sequence having at least 80% homology thereto, and (b) culturing the cell from step (a) such that polypeptides encoded by the artificial DNA molecule are expressed, thereby synthesizing 5-MeO-DMT.

In some embodiments, the method comprises (the steps of): (a) providing a cell comprising an artificial DNA molecule comprising: (i) SEQ ID NO: 1 or 2; and (ii) a nucleic acid sequence encoding PsiH; or a nucleic acid sequence having at least 80% homology thereto, and (b) culturing the cell from step (a) such that polypeptides encoded by the artificial DNA molecule are expressed, thereby synthesizing 4-hydroxytryptamine (4-HT).

In some embodiments, the method comprises (the steps of): (a) providing a cell comprising an artificial DNA molecule comprising: (i) SEQ ID NO: 1 or 2; (ii) a nucleic acid sequence encoding PsiH; and (iii) SEQ ID NO: 10, 3, or 7 or a nucleic acid sequence encoding PsiM; or a nucleic acid sequence having at least 80% homology thereto, and (b) culturing the cell from step (a) such that polypeptides encoded by the artificial DNA molecule are expressed, thereby synthesizing Norpsilocin. In some embodiments, the method comprises (the steps of): (a) providing a cell comprising an artificial DNA molecule comprising: (i) SEQ ID NO: 1 or 2; (ii) a nucleic acid sequence encoding PsiH; and (iii) SEQ ID NO: 10 or 3; or a nucleic acid sequence having at least 80% homology thereto, and (b) culturing the cell from step (a) such that polypeptides encoded by the artificial DNA molecule are expressed, thereby synthesizing Norpsilocin.

In some embodiments, the method comprises (the steps of): (a) providing a cell comprising an artificial DNA molecule comprising: (i) SEQ ID NO: 1 or 2; (ii) a nucleic acid sequence encoding PsiH; and (iii) SEQ ID NO: 10, 3, or 7 or a nucleic acid sequence encoding PsiM; or a nucleic acid sequence having at least 80% homology thereto, and (b) culturing the cell from step (a) such that polypeptides encoded by the artificial DNA molecule are expressed, thereby synthesizing Psilocin. In some embodiments, the method comprises (the steps of): (a) providing a cell comprising an artificial DNA molecule comprising: (i) SEQ ID NO: 1 or 2; (ii) a nucleic acid sequence encoding PsiH; and (iii) SEQ ID NO: 10 or 3; or a nucleic acid sequence having at least 80% homology thereto, and (b) culturing the cell from step (a) such that polypeptides encoded by the artificial DNA molecule are expressed, thereby synthesizing Psilocin. In some embodiments, the method comprises (the steps of): (a) providing a cell comprising an artificial DNA molecule comprising: (i) SEQ ID NO: 1 or 2; (ii) SEQ ID NO: 3, 7, or 10; and (iii) a nucleic acid sequence encoding PsiH; or a nucleic acid sequence having at least 80% homology thereto, and (b) culturing the cell from step (a) such that polypeptides encoded by the artificial DNA molecule are expressed, thereby synthesizing Psilocin. In some embodiments, the method comprises (the steps of): (a) providing a cell comprising an artificial DNA molecule comprising: (i) SEQ ID NO: 1 or 2; (ii) SEQ ID NO: 3 or 7; and (iii) a nucleic acid sequence encoding PsiH; or a nucleic acid sequence having at least 80% homology thereto, and (b) culturing the cell from step (a) such that polypeptides encoded by the artificial DNA molecule are expressed, thereby synthesizing Psilocin.

In some embodiments, the method comprises (the steps of): (a) providing a cell comprising an artificial DNA molecule comprising: (i) SEQ ID NO: 1 or 2; (ii) a nucleic acid sequence encoding PsiH; and a nucleic acid sequence encoding PsiK; or a nucleic acid sequence having at least 80% homology thereto, and (b) culturing the cell from step (a) such that polypeptides encoded by the artificial DNA molecule are expressed, thereby synthesizing Norebaeocystin.

In some embodiments, the method comprises (the steps of): (a) providing a cell comprising an artificial DNA molecule comprising: (i) SEQ ID NO: 1 or 2; (ii) a nucleic acid sequence encoding PsiH; (iii) a nucleic acid sequence encoding PsiK; and (iv) a nucleic acid sequence encoding PsiM or SEQ ID NO: 10, 3, or 7; or a nucleic acid sequence having at least 80% homology thereto, and (b) culturing the cell from step (a) such that polypeptides encoded by the artificial DNA molecule are expressed, thereby synthesizing Baeocystin. In some embodiments, the method comprises (the steps of): (a) providing a cell comprising an artificial DNA molecule comprising: (i) SEQ ID NO: 1 or 2; (ii) a nucleic acid sequence encoding PsiH; (iii) a nucleic acid sequence encoding PsiK; and (iv) a nucleic acid sequence encoding PsiM; or a nucleic acid sequence having at least 80% homology thereto, and (b) culturing the cell from step (a) such that polypeptides encoded by the artificial DNA molecule are expressed, thereby synthesizing Baeocystin. In some embodiments, the method comprises (the steps of): (a) providing a cell comprising an artificial DNA molecule comprising: (i) SEQ ID NO: 1 or 2; (ii) a nucleic acid sequence encoding PsiH; (iii) SEQ ID NO: 10, 7, or 3, or a nucleic acid sequence encoding PsiM; and (iv) a nucleic acid sequence encoding PsiK; or a nucleic acid sequence having at least 80% homology thereto, and (b) culturing the cell from step (a) such that polypeptides encoded by the artificial DNA molecule are expressed, thereby synthesizing Baeocystin. In some embodiments, the method comprises (the steps of): (a) providing a cell comprising an artificial DNA molecule comprising: (i) SEQ ID NO: 1 or 2; (ii) a nucleic acid sequence encoding PsiH; (iii) SEQ ID NO: 10 or 3; and (iv) a nucleic acid sequence encoding PsiK; or a nucleic acid sequence having at least 80% homology thereto, and (b) culturing the cell from step (a) such that polypeptides encoded by the artificial DNA molecule are expressed, thereby synthesizing Baeocystin.

In some embodiments, the method comprises (the steps of): (a) providing a cell comprising an artificial DNA molecule comprising: (i) SEQ ID NO: 1 or 2; (ii) a nucleic acid sequence encoding PsiH; (iii) a nucleic acid sequence encoding PsiK; and (iv) a nucleic acid sequence encoding PsiM or SEQ ID NO: 10, 3, or 7; or a nucleic acid sequence having at least 80% homology thereto, and (b) culturing the cell from step (a) such that polypeptides encoded by the artificial DNA molecule are expressed, thereby synthesizing Psilocybin. In some embodiments, the method comprises (the steps of): (a) providing a cell comprising an artificial DNA molecule comprising: (i) SEQ ID NO: 1 or 2; (ii) a nucleic acid sequence encoding PsiH; (iii) a nucleic acid sequence encoding PsiK; and (iv) a nucleic acid sequence encoding PsiM; or a nucleic acid sequence having at least 80% homology thereto, and (b) culturing the cell from step (a) such that polypeptides encoded by the artificial DNA molecule are expressed, thereby synthesizing Psilocybin. In some embodiments, the method comprises (the steps of): (a) providing a cell comprising an artificial DNA molecule comprising: (i) SEQ ID NO: 1 or 2; (ii) a nucleic acid sequence encoding PsiH; (iii) SEQ ID NO: 10, 7, or 3, or a nucleic acid sequence encoding PsiM; and (iv) a nucleic acid sequence encoding PsiK; or a nucleic acid sequence having at least 80% homology thereto, and (b) culturing the cell from step (a) such that polypeptides encoded by the artificial DNA molecule are expressed, thereby synthesizing Psilocybin. In some embodiments, the method comprises (the steps of): (a) providing a cell comprising an artificial DNA molecule comprising: (i) SEQ ID NO: 1 or 2; (ii) a nucleic acid sequence encoding PsiH; (iii) SEQ ID NO: 10 or 3; and (iv) a nucleic acid sequence encoding PsiK; or a nucleic acid sequence having at least 80% homology thereto, and (b) culturing the cell from step (a) such that polypeptides encoded by the artificial DNA molecule are expressed, thereby synthesizing Psilocybin.

In some embodiments, the method comprises (the steps of): (a) providing a cell comprising an artificial DNA molecule comprising: (i) SEQ ID NO: 1 or 2; (ii) a nucleic acid sequence encoding PsiH; (iii) SEQ ID NO: 10, 3, or 7 or a nucleic acid sequence encoding PsiM; and (iv) a nucleic acid sequence encoding PsiK; or a nucleic acid sequence having at least 80% homology thereto, and (b) culturing the cell from step (a) such that polypeptides encoded by the artificial DNA molecule are expressed, thereby synthesizing Psilocybin. In some embodiments, the method comprises (the steps of): (a) providing a cell comprising an artificial DNA molecule comprising: (i) SEQ ID NO: 1 or 2; (ii) a nucleic acid sequence encoding PsiH; (iii) SEQ ID NO: 10 or 3; and (iv) a nucleic acid sequence encoding PsiK; or a nucleic acid sequence having at least 80% homology thereto, and (b) culturing the cell from step (a) such that polypeptides encoded by the artificial DNA molecule are expressed, thereby synthesizing Psilocybin. In some embodiments, the method comprises (the steps of): (a) providing a cell comprising an artificial DNA molecule comprising: (i) SEQ ID NO: 1 or 2; (ii) SEQ ID NO: 3, 7, or 10; (iii) a nucleic acid sequence encoding PsiH; and (iv) a nucleic acid sequence encoding PsiK; or a nucleic acid sequence having at least 80% homology thereto, and (b) culturing the cell from step (a) such that polypeptides encoded by the artificial DNA molecule are expressed, thereby synthesizing Psilocybin. In some embodiments, the method comprises (the steps of): (a) providing a cell comprising an artificial DNA molecule comprising: (i) SEQ ID NO: 1 or 2; (ii) SEQ ID NO: 3 or 7; (iii) a nucleic acid sequence encoding PsiH; and (iv) a nucleic acid sequence encoding PsiK; or a nucleic acid sequence having at least 80% homology thereto, and (b) culturing the cell from step (a) such that polypeptides encoded by the artificial DNA molecule are expressed, thereby synthesizing Psilocybin.

According to another aspect, there is provided a method for synthesizing tryptamine comprising contacting tryptophan with an effective amount of a recombinant polypeptide comprising an amino acid sequence having at least 84% homology or identity to any one of SEQ ID Nos: 4-5, thereby synthesizing tryptamine.

According to another aspect, there is provided a method for synthesizing NMT comprising contacting Tryptamine with an effective amount of a recombinant polypeptide comprising an amino acid sequence having at least 69% homology or identity to any one of SEQ ID Nos: 6, 8-9, or a combination thereof, thereby synthesizing NMT.

According to another aspect, there is provided a method for synthesizing DMT comprising contacting NMT with an effective amount of a recombinant polypeptide comprising an amino acid sequence having at least 69% homology or identity to any one of SEQ ID Nos: 6, 8-9, or a combination thereof, thereby synthesizing DMT.

According to another aspect, there is a method for synthesizing N-Me-Serotonin comprising contacting Serotonin with an effective amount of a recombinant protein comprising an amino acid sequence having at least 69% homology or identity to any one of SEQ ID Nos: 6, 8-9, or a combination thereof, thereby synthesizing N-Me-Serotonin.

According to another aspect, there is a method for synthesizing Bufotenine comprising contacting N-Me-Serotonin with an effective amount of a recombinant polypeptide comprising an amino acid sequence having at least 69% homology or identity to any one of SEQ ID Nos: 6, 8-9, or a combination thereof, thereby synthesizing Bufotenine.

According to another aspect, there is a method for synthesizing 5-MeO-NMT comprising contacting 5-MeO-tryptamine with an effective amount of a recombinant polypeptide comprising an amino acid sequence having at least 69% homology or identity to any one of SEQ ID Nos: 6, 8-9, or a combination thereof, thereby synthesizing 5-MeO-NMT.

According to another aspect, there is a method for synthesizing 5-MeO-DMT comprising contacting 5-MeO-NMT with an effective amount of a recombinant polypeptide comprising an amino acid sequence having at least 69% homology or identity to any one of SEQ ID Nos: 6, 8-9, or a combination thereof, thereby synthesizing 5-MeO-DMT.

According to another aspect, there is a method for synthesizing Norpsilocin comprising contacting 4-OH-Tryptamine with an effective amount of a recombinant polypeptide comprising an amino acid sequence having at least 69% homology or identity to any one of SEQ ID Nos: 6, 8-9, or a combination thereof, thereby synthesizing Norpsilocin.

According to another aspect, there is a method for synthesizing Psilocin comprising contacting Norpsilocin with an effective amount of a recombinant polypeptide comprising an amino acid sequence having at least 69% homology or identity to any one of SEQ ID Nos: 6, 8-9, or a combination thereof, thereby synthesizing Psilocin.

According to another aspect, there is a method for synthesizing Baeocystin comprising contacting Norbaeocystin with an effective amount of a recombinant polypeptide comprising an amino acid sequence having at least 69% homology or identity to any one of SEQ ID Nos: 6, 8-9, or a combination thereof, thereby synthesizing Baeocystin.

According to another aspect, there is a method for synthesizing Psilocybin comprising contacting Baeocystin with an effective amount of a recombinant polypeptide comprising an amino acid sequence having at least 69% homology or identity to any one of SEQ ID Nos: 6, 8-9, or a combination thereof, thereby synthesizing Psilocybin.

According to another aspect, there is provided a method for synthesizing 5-MeO-tryptamine comprising contacting Serotonin with an effective amount of a recombinant protein comprising an amino acid sequence having at least 90% homology or identity to any one of SEQ ID Nos: 12 or 14, thereby synthesizing 5-MeO-tryptamine.

According to another aspect, there is provided a method for synthesizing 5-MeO-NMT comprising contacting N-Me-Serotonin with an effective amount of a recombinant protein comprising an amino acid sequence having at least 90% homology or identity to any one of SEQ ID Nos: 12 or 14, thereby synthesizing 5-MeO-NMT.

According to another aspect, there is provided a method for synthesizing 5-MeO-DMT comprising contacting Bufotenine with an effective amount of a recombinant protein comprising an amino acid sequence having at least 90% homology or identity to any one of SEQ ID Nos: 12 or 14, thereby synthesizing 5-MeO-DMT.

In some embodiments, the method further comprises a step before step (a), comprising introducing or transfecting the cell with a vector.

Methods for introducing or transfecting a cell with a vector (e.g., plasmid and/or agrobacterium) are common and would be apparent to one of ordinary skill in the art.

In some embodiments, introducing or transfecting comprises transferring a vector comprising the artificial DNA molecule of the invention into a cell; or modifying the genome of a cell to include the artificial DNA molecule of the invention. In some embodiments, the transferring comprises transfection. In some embodiments, the transferring comprises transformation. In some embodiments, the transferring comprises lipofection. In some embodiments, the transferring comprises nucleofection. In some embodiments, the transferring comprises viral infection.

In some embodiments, vector comprises or is a plasmid, an agrobacterium, an expression vector, or any combination thereof.

As used herein, the terms “transfecting” and “introducing” are interchangeable.

In some embodiments, contacting is in a cell-free system.

According to some embodiments, there is provided a method for obtaining an extract from a transgenic cell or a transfected cell.

In some embodiments, the method comprises culturing a transgenic cell or a transfected cell in a medium and extracting the transgenic cell or the transfected cell.

In some embodiments, the method comprises the steps: (a) culturing a transgenic cell or a transfected cell in a medium; and (b) extracting the transgenic cell or the transfected cell, thereby obtaining an extract from the transgenic cell or the transfected cell.

In some embodiments, the transgenic cell or the transfected cell is the transgenic cell disclosed herein.

In some embodiments, the transgenic cell or the transfected cell comprises the artificial DNA molecule of the invention or a plurality thereof, as disclosed herein.

As used herein, the term “plurality” refers to any integer being equal to or greater than 2.

In some embodiments, the transgenic cell or the transfected cell comprises the vector, and/or plasmid or agrobacterium as disclosed herein.

In some embodiments, the cell is a transgenic cell, or a cell transfected with a artificial DNA molecule, as disclosed herein.

In some embodiments, the method further comprises a step comprising separating the cultured transgenic cell or the cultured transfected cell from the medium.

Methods for separating cell from a medium are common and may include, but not limited to, centrifugation, ultracentrifugation, or other, as would be apparent to one of ordinary skill in the art.

According to some embodiments, there is provided an extract of a transgenic cell or a transfected cell obtained according to the herein disclosed method.

According to some embodiments, there is provided a medium or a portion thereof separated from a cultured transgenic cell or a cultured transfected cell, obtained according to the herein disclosed method.

According to some embodiments, there is provided a composition comprising: (a) the extract disclosed herein; (b) the medium disclosed herein or a portion thereof; or (c) any combination of (a) and (b), and an acceptable carrier, as described herein.

In some embodiments, a portion comprises a fraction or a plurality thereof.

### Polynucleotides, Proteins, Cells, and Compositions

The present invention, in some embodiments, is directed to an artificial DNA molecule, including a polypeptide encoded by the same, that are involved in production/or synthesis of tryptamine and/or at least one metabolite thereof, and methods of using same in the production or synthesis of tryptamine and/or at least one metabolite thereof.

In some embodiments, the polynucleotide is an isolated polynucleotide. In some embodiments, the polynucleotide is a DNA molecule. In some embodiments, the polynucleotide is an isolated DNA molecule. In some embodiments, the DNA molecule is an isolated DNA molecule. In some embodiments, the DNA molecule is a complementary DNA (cDNA) molecule. In some embodiments, the isolated polynucleotide is an artificial DNA molecule. The terms “polynucleotide” and “artificial DNA molecule” are used herein interchangeably.

As used herein, the terms “isolated polynucleotide” and “isolated DNA molecule” refers to a nucleic acid molecule that is essentially free from contaminating cellular components, such as carbohydrate, lipid, or other proteinaceous impurities associated with the nucleic acid in nature. Typically, a preparation of isolated DNA or RNA contains the nucleic acid in a highly purified form, e.g., at least about 80% pure, at least about 90% pure, at least about 95% pure, greater than 95% pure, or greater than 99% pure. In some embodiments, the isolated polynucleotide is any one of DNA, RNA, and cDNA. In some embodiments, the isolated polynucleotide is a synthesized polynucleotide. Synthesis of polynucleotides is well known in the art and may be performed, for example, by ligating or covalently linking multiple nucleic acid molecules together by primer linkers.

The term “nucleic acid” is well known in the art. A “nucleic acid” as used herein will generally refer to any molecule (e.g., a strand) of DNA, RNA or a derivative or analog thereof, comprising nucleotides. Nucleotides are comprised of nucleosides and phosphate groups. The nitrogenous bases of nucleosides include, for example, naturally occurring purine or pyrimidine nucleosides as found in DNA (e.g., an adenine “A,” a guanine “G,” a thymine “T” or a cytosine “C”) or RNA (e.g., an A, a G, an uracil “U” or a C).

The term “nucleic acid molecule” includes but is not limited to single-stranded RNA (ssRNA), double-stranded RNA (dsRNA), single-stranded DNA (ssDNA), double-stranded DNA (dsDNA), small RNAs, circular nucleic acids, fragments of genomic DNA or RNA, degraded nucleic acids, amplification products, modified nucleic acids, plasmid or organellar nucleic acids, and artificial nucleic acids such as oligonucleotides.

In some embodiments, the polynucleotide comprises or consists of the nucleic acid sequence set forth in SEQ ID NO: 1.

In some embodiments, the polynucleotide comprises a nucleic acid sequence with at least 80%, at least 85%, at least 87%, at least 95%, or at least 99% homology or identity to SEQ ID NO: 1, or any value and range therebetween. Each possibility represents a separate embodiment of the invention. In some embodiments, the polynucleotide comprises a nucleic acid sequence with 80% to 100%, 88% to 100%, 90% to 100%, or 95% to 100% homology or identity to SEQ ID NO: 1. Each possibility represents a separate embodiment of the invention. In some embodiments, TDC1 or PvTDC1, as disclosed herein, comprises or consists of the nucleic acid sequence set forth in SEQ ID NO: 1.

In some embodiments, the polynucleotide comprises or consists of the nucleic acid sequence set forth in SEQ ID NO: 2.

In some embodiments, the polynucleotide comprises a nucleic acid sequence with at least 80%, at least 85%, at least 87%, at least 95%, or at least 99% homology or identity to SEQ ID NO: 2, or any value and range therebetween. Each possibility represents a separate embodiment of the invention. In some embodiments, the polynucleotide comprises a nucleic acid sequence with 80% to 100%, 88% to 100%, 90% to 100%, or 95% to 100% homology or identity to SEQ ID NO: 2. Each possibility represents a separate embodiment of the invention. In some embodiments, TDC2 or PvTDC2, as disclosed herein, comprises or consists of the nucleic acid sequence set forth in SEQ ID NO: 2.

In some embodiments, the polynucleotide comprises or consists of the nucleic acid sequence set forth in SEQ ID NO: 3.

In some embodiments, the polynucleotide comprises a nucleic acid sequence with at least 80%, at least 85%, at least 87%, at least 95%, or at least 99% homology or identity to SEQ ID NO: 3, or any value and range therebetween. Each possibility represents a separate embodiment of the invention. In some embodiments, the polynucleotide comprises a nucleic acid sequence with 80% to 100%, 88% to 100%, 90% to 100%, or 95% to 100% homology or identity to SEQ ID NO: 3. Each possibility represents a separate embodiment of the invention. In some embodiments, NMT or PvNMT, as disclosed herein, comprises or consists of the nucleic acid sequence set forth in SEQ ID NO: 3.

In some embodiments, the polynucleotide comprises a nucleic acid sequence with at least 80%, at least 85%, at least 87%, at least 95%, or at least 99% homology or identity to SEQ ID NO: 7, or any value and range therebetween. Each possibility represents a separate embodiment of the invention. In some embodiments, the polynucleotide comprises a nucleic acid sequence with 80% to 100%, 88% to 100%, 90% to 100%, or 95% to 100% homology or identity to SEQ ID NO: 7. Each possibility represents a separate embodiment of the invention. In some embodiments, NMT or AaNMT, as disclosed herein, comprises or consists of the nucleic acid sequence set forth in SEQ ID NO: 7.

In some embodiments, the polynucleotide comprises a nucleic acid sequence with at least 80%, at least 85%, at least 87%, at least 95%, or at least 99% homology or identity to SEQ ID NO: 10, or any value and range therebetween. Each possibility represents a separate embodiment of the invention. In some embodiments, the polynucleotide comprises a nucleic acid sequence with 80% to 100%, 88% to 100%, 90% to 100%, or 95% to 100% homology or identity to SEQ ID NO: 10. Each possibility represents a separate embodiment of the invention. In some embodiments, NMT or RmNMT, as disclosed herein, comprises or consists of the nucleic acid sequence set forth in SEQ ID NO: 10.

In some embodiments, the polynucleotide comprises a nucleic acid sequence with at least 80%, at least 85%, at least 87%, at least 95%, or at least 99% homology or identity to SEQ ID NO: 11, or any value and range therebetween. Each possibility represents a separate embodiment of the invention. In some embodiments, the polynucleotide comprises a nucleic acid sequence with 80% to 100%, 88% to 100%, 90% to 100%, or 95% to 100% homology or identity to SEQ ID NO: 11. Each possibility represents a separate embodiment of the invention. In some embodiments, OMT, COMT, or AtOMT, as disclosed herein, comprises or consists of the nucleic acid sequence set forth in SEQ ID NO: 11. In some embodiments, OMT, COMT, or AtOMT comprises a mutant thereof. In some embodiments, the mutant is characterized by increased activity compared to SEQ ID NO: 11. In some embodiments, the activity comprises synthesis of 5-MeO-NMT, 5-MeO-DMT, or both.

In some embodiments, synthesis of 5-MeO-DMT is increased by at least 5-fold, 7-fold, 15-fold, 20-fold, 25-fold, 30-fold, 35-fold, 40-fold, or 50-fold, by a polypeptide encoded by the mutant compared to a polypeptide encoded by SEQ ID NO: 11, or any value and range therebetween. Each possibility represents a separate embodiment of the invention.

In some embodiments, the polynucleotide comprises a mutant or a mutated sequence of SEQ ID NO: 11. In some embodiments, the mutant encodes a polypeptide comprising a single amino acid substitution compared to a polypeptide encoded from SEQ ID NO: 11. In some embodiments, the mutation is a substitution of Alanine in position 160 of a polypeptide encoded from SEQ ID NO: 11 to Glycine. In some embodiments, the mutant encodes a polypeptide comprising Glycine in position 160, wherein a polypeptide encoded from SEQ ID NO: 11 comprises Alaine in position 160. In some embodiments, the mutant is encoded by a polypeptide comprising a nucleic acid sequence with at least 80%, at least 85%, at least 87%, at least 95%, or at least 99% homology or identity to SEQ ID NO: 13, or any value and range therebetween. Each possibility represents a separate embodiment of the invention. In some embodiments, the mutant is encoded by a polypeptide comprising a nucleic acid sequence with 80% to 100%, 88% to 100%, 90% to 100%, or 95% to 100% homology or identity to SEQ ID NO: 13. Each possibility represents a separate embodiment of the invention.

In some embodiments, the (AtOMT) mutant is encoded by the nucleic acid sequence set forth in SEQ ID NO: 13. In some embodiments, the mutant comprises any mutant of SEQ ID NO: 11 as long as it encodes a polypeptide having an increased OMT activity as disclosed herein. In some embodiments, the mutant comprises any mutant of SEQ ID NO: 11 as long as it encodes a polypeptide comprising a substitution of Glycine 160 to Alanine. In some embodiments, the mutant comprises any mutant of SEQ ID NO: 11 as long as it encodes a polypeptide comprising a substitution of Glycine 160 to Alanine and is characterized by an increased OMT activity as disclosed herein.

In some embodiments, the polynucleotide of the invention comprises 900 to 1,700 nucleotides. In some embodiments, the polynucleotide of the invention is 900 to 1,700 nucleotides long.

In some embodiments, 900 to 1,700 nucleotides comprises at least 900 nucleotides, at least 1,000 nucleotides, at least 1,200 nucleotides, at least 1,30 nucleotides, at least 1,400 nucleotides, at least 1,500 nucleotides, or at least 1,690 nucleotides, or any value and range therebetween. Each possibility represents a separate embodiment of the invention. In some embodiments, 900 to 1,700 nucleotides comprises: 950 to 1,700 nucleotides, 1,200 to 1,700 nucleotides, 1,400 to 1,700 nucleotides, 1,450 to 1,700 nucleotides, or 1,500 to 1,700 nucleotides. Each possibility represents a separate embodiment of the invention.

In some embodiments, the polynucleotide encodes a polypeptide characterized by decarboxylating activity. In some embodiments, the polynucleotide encodes a polypeptide characterized by having an activity of releasing a carboxyl group from a substrate, e.g., releasing CO₂. In some embodiments, the polynucleotide encodes a polypeptide being a decarboxylase. In some embodiments, the decarboxylase is a decarboxylase derived from a plant. In some embodiments, the plant is Psychotria viridis and/or Psychotria carthagenensis. In some embodiments, the polynucleotide encoding a decarboxylating polypeptide is selected from SEQ ID Nos: 1-2, an analog thereof having at least 70%, 80%, 90%, or 95% homology or identity thereto, or any combination thereof.

In some embodiments, the polynucleotide encodes a polypeptide characterized by methyltransferring activity. In some embodiments, the polynucleotide encodes a polypeptide characterized by having an activity of transferring a methyl group to a substrate. In some embodiments, the polynucleotide encodes a polypeptide being a methyltransferase. In some embodiments, the methyltransferase is an N-methyltransferase. In some embodiments, the methyltransferase is derived from a plant. In some embodiments, the methyltransferase is derived from a animal. In some embodiments, the animal is an amphibian. In some embodiments, the amphibian is a toad. In some embodiments, the toad is Rhinella marina. In some embodiments, the plant is Psychotria viridis and/or Psychotria carthagenensis and/or Acacia acuminata. In some embodiments, the polynucleotide encoding a methyltransferring polypeptide comprises a nucleic acid sequence as set forth in any one of SEQ ID Nos: 3, 7, 10, an analog thereof having at least 70%, 80%, 90%, or 95% homology or identity thereto, or any combination thereof.

According to some embodiments, there is provided a vector comprising the polynucleotide/artificial DNA molecule disclosed herein.

In some embodiments, the vector comprises a plasmid. In some embodiments, the vector comprises or is an agrobacterium comprising the artificial DNA molecule. In some embodiments, the vector is an expression vector. In some embodiments, the vector is a plant expression vector. In some embodiments, the vector is for use in expressing a decarboxylase, hydroxylase, methyl transferase, or any combination thereof, encoding nucleic acid sequence(s) as disclosed herein. In some embodiments, the vector is for use in heterologous expression of a decarboxylase, methyl transferase, or both, encoding nucleic acid sequence(s) as disclosed herein in a cell, a tissue, or an organism. In some embodiments, the vector is for use in producing or the production of tryptamine and/or at least one metabolite thereof, in a cell, a tissue, or an organism.

In some embodiments, the vector further comprises at least one additional nucleic acid sequence. In some embodiments, the at least one additional nucleic acid encods at least one enzyme selected from: CYP71P1, O-methyltransferase (OMT), PsiH, PsiK, PsiM, and or combination thereof.

Expressing a polynucleotide within a cell is well known to one skilled in the art. It can be carried out by, among many methods, transfection, viral infection, or direct alteration of the cell's genome. In some embodiments, the polynucleotide is in an expression vector such as plasmid or viral vector. A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector, a virgaviridae viral vector, or a poxviral vector. The barley stripe mosaic virus (BSMV), the tobacco rattle virus and the cabbage leaf curl geminivirus (CbLCV) may also be used. The promoters may be active in plant cells. The promoters may be a viral promoter.

In some embodiments, the polynucleotide as disclosed herein is operably linked to a promoter. The term “operably linked” is intended to mean that the nucleotide sequence of interest is linked to the regulatory element or elements in a manner that allows for expression of the nucleotide sequence (e.g., in an in vitro transcription/translation system or in a host cell when the vector is introduced into the host cell). In some embodiments, the promoter is operably linked to the polynucleotide of the invention. In some embodiments, the promoter is a heterologous promoter. In some embodiments, the promoter is the endogenous promoter.

In some embodiments, the vector is introduced into the cell by standard methods including electroporation (e.g., as described in From et al., Proc. Natl. Acad. Sci. USA 82, 5824 (1985)), heat shock, infection by viral vectors, high velocity ballistic penetration by small particles with the nucleic acid either within the matrix of small beads or particles, or on the surface (Klein et al., Nature 327. 70-73 (1987)), such as biolistic use of coated particles, and needle-like particles, Agrobacterium Ti plasmids and/or the like.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins. The promoter may extend upstream or downstream of the transcriptional start site and may be any size ranging from a few base pairs to several kilo-bases.

In some embodiments, the polynucleotide is transcribed by RNA polymerase II (RNAP II and Pol II). RNAP II is an enzyme found in eukaryotic cells, known to catalyze the transcription of DNA to synthesize precursors of mRNA and most snRNA and microRNA.

In some embodiments, a plant expression vector is used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hspl7.5-E or hspl7.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-IMTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as systemic infection and targeting specificity, are used for in vivo expression. In one embodiment, systemic infection is inherent in the life cycle of, for example, the retrovirus and is the process by which a single infected cell produces many progeny virions that infect neighboring cells. In one embodiment, the result is that a large area becomes rapidly infected, most of which was not initially infected by the original viral particles. In one embodiment, viral vectors are produced that are unable to spread systemically. In one embodiment, this characteristic can be useful if the desired purpose is to introduce a specified gene into only a localized number of targeted cells.

In some embodiments, plant viral vectors are used. In some embodiments, a wild-type virus is used. In some embodiments, a deconstructed virus such as are known in the art is used. In some embodiments, Agrobacterium is used to introduce the vector of the invention into a virus.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation, agrobacterium Ti plasmids and infection with recombinant viral vectors. In addition, see U.S. Pat. Nos. 5,464,764 and 5,487,992 for positive-negative selection methods.

It will be appreciated that other than containing the necessary elements for the transcription and translation of the inserted coding sequence (encoding the polypeptide), the expression construct of the present invention can also include sequences engineered to optimize stability, production, purification, yield, or activity of the expressed polypeptide.

In some embodiments, the vector comprises a polynucleotide/artificial DNA molecule encoding a polypeptide (such as a recombinant polypeptide) comprising an amino acid sequence as described herein.

In some embodiments, the polypeptide comprises or consists of the amino acid sequence set forth in SEQ ID NO: 4.

In some embodiments, the polypeptide comprises an amino acid sequence having at least 85%, at least 92%, at least 95%, or at least 99% homology or identity to SEQ ID NO: 4, or any value and range therebetween. Each possibility represents a separate embodiment of the invention. In some embodiments, the protein comprises an amino acid sequence with 85% to 100%, 90% to 100%, or 95% to 100% homology or identity to SEQ ID NO: 4. Each possibility represents a separate embodiment of the invention. In some embodiments, TDC1 or PvTDC1, as disclosed herein, comprises or consists of the amino acid sequence set forth in SEQ ID NO: 4.

In some embodiments, the polypeptide comprises or consists of the amino acid sequence set forth in SEQ ID NO: 5.

In some embodiments, the polypeptide comprises an amino acid sequence having at least 84%, at least 92%, at least 95%, or at least 99% homology or identity to SEQ ID NO: 5, or any value and range therebetween. Each possibility represents a separate embodiment of the invention. In some embodiments, the protein comprises an amino acid sequence with 84% to 100%, 90% to 100%, or 95% to 100% homology or identity to SEQ ID NO: 5. Each possibility represents a separate embodiment of the invention. In some embodiments, TDC2 or PvTDC2, as disclosed herein, comprises or consists of the amino acid sequence set forth in SEQ ID NO: 5.

In some embodiments, the polypeptide comprises or consists of the amino acid sequence set forth in SEQ ID NO: 6.

In some embodiments, the polypeptide comprises an amino acid sequence having at least 69%, at least 80%, at least 90%, or at least 99% homology or identity to SEQ ID NO: 6, or any value and range therebetween. Each possibility represents a separate embodiment of the invention. In some embodiments, the protein comprises an amino acid sequence with 69% to 100%, 85% to 100%, or 90% to 100% homology or identity to SEQ ID NO: 6. Each possibility represents a separate embodiment of the invention. In some embodiments, NMT1 or PvNMT1, as disclosed herein, comprises or consists of the amino acid sequence set forth in SEQ ID NO: 6.

In some embodiments, the polypeptide comprises or consists of the amino acid sequence set forth in SEQ ID NO: 8.

In some embodiments, the polypeptide comprises an amino acid sequence having at least 69%, at least 80%, at least 90%, or at least 99% homology or identity to SEQ ID NO: 8, or any value and range therebetween. Each possibility represents a separate embodiment of the invention. In some embodiments, the protein comprises an amino acid sequence with 69% to 100%, 85% to 100%, or 90% to 100% homology or identity to SEQ ID NO: 8. Each possibility represents a separate embodiment of the invention. In some embodiments, NMT1 or AaNMT1, as disclosed herein, comprises or consists of the amino acid sequence set forth in SEQ ID NO: 8.

In some embodiments, the polypeptide comprises or consists of the amino acid sequence set forth in SEQ ID NO: 9.

In some embodiments, the polypeptide comprises an amino acid sequence having at least 69%, at least 80%, at least 90%, or at least 99% homology or identity to SEQ ID NO: 9, or any value and range therebetween. Each possibility represents a separate embodiment of the invention. In some embodiments, the protein comprises an amino acid sequence with 69% to 100%, 85% to 100%, or 90% to 100% homology or identity to SEQ ID NO: 9. Each possibility represents a separate embodiment of the invention. In some embodiments, NMT and/or NM1 and/or RmNMT1 and/or RmNMT, as disclosed herein, comprises or consists of the amino acid sequence set forth in SEQ ID NO: 9.

In some embodiments, the polypeptide comprises or consists of the amino acid sequence set forth in SEQ ID NO: 12 or a mutant thereof.

In some embodiments, the polypeptide comprises an amino acid sequence having at least at least 80%, at least 90%, or at least 99% homology or identity to SEQ ID NO: 12, or any value and range therebetween. Each possibility represents a separate embodiment of the invention. In some embodiments, the protein comprises an amino acid sequence with 80% to 100%, 85% to 100%, or 90% to 100% homology or identity to SEQ ID NO: 12. Each possibility represents a separate embodiment of the invention. In some embodiments, OMT and/or COMT and/or AtOMT and/or AtCOMT, as disclosed herein, comprises or consists of the amino acid sequence set forth in SEQ ID NO: 12.

In some embodiments, the polypeptide comprises a substitution of Alanine in position 160 of SEQ ID NO: 12 to Glycine. In some embodiments, the polypeptide comprising a substitution of Alanine in position 160 of SEQ ID NO: 12 to Glycine, is or comprises SEQ ID NO: 14. In some embodiments, a mutant OMT and/or COMT and/or AtOMT and/or AtCOMT, as disclosed herein, comprises or consists of the amino acid sequence set forth in SEQ ID NO: 14. In some embodiments, a mutant OMT and/or COMT and/or AtOMT and/or AtCOMT, as disclosed herein, comprising or consisting of the amino acid sequence set forth in SEQ ID NO: 14, is characterized by increased activity compared to a control. In some embodiments, activity comprises synthesis of 5-MeO-NMT, 5-MeO-DMT, or both. In some embodiments, increased is by at least 5-fold, 7-fold, 10-fold, 15-fold, 20-fold, 25-fold, 30-fold, 35-fold, 40-fold, 45-fold, 50-fold, or any value and range therebetween, compared to a control. Each possibility represents a separate embodiment of the invention. In some embodiments, a control comprises or consists of a wild-type OMT and/or COMT and/or AtOMT and/or AtCOMT. In some embodiments, a control comprises or consists of OMT and/or COMT and/or AtOMT and/or AtCOMT encoded by SEQ ID NO: 11. In some embodiments, a control comprises a polypeptide comprising an amino acid sequence set forth in SEQ ID NO: 12.

In some embodiments, the polypeptide comprising an amino acid sequence selected from SEQ ID Nos: 4-5, or an analog thereof comprising 85-100% homology or identity thereto is characterized by decarboxylating activity, as described herein. In some embodiments, the polypeptide is characterized by having an activity of releasing a carboxyl group from a substrate (such as resulting with the release of CO₂). In some embodiments, decarboxylase or decarboxylating activity is tryptophan decarboxylase or decarboxylating tryptophan, e.g., removing or releasing a carboxyl group from tryptophan.

In some embodiments, the polypeptide comprising any one of the amino acid sequences as set forth in SEQ ID Nos: 6, 8-9, an analog thereof comprising 69-100% homology or identity thereto, or any combination thereof, is characterized by methyl transferring, as described herein. In some embodiments, the polypeptide is characterized by having an activity of transferring a methyl group to a substrate. In some embodiments, methyltransferase or methyl transferring activity is N-methyl transferase or N-methylation.

In some embodiments, the polypeptide comprising an amino acid sequence selected from SEQ ID Nos: 12, 14, or an analog thereof comprising 85-100% homology or identity thereto is characterized by methyl transferring, as described herein. In some embodiments, the polypeptide is characterized by having an activity of transferring a methyl group to a substrate. In some embodiments, methyltransferase or methyl transferring activity is O-methyl transferase or O-methylation.

In some embodiments, the polypeptide is an isolated polypeptide. In some embodiments, the polypeptide is a recombinant polypeptide. In some embodiments, a polypeptide of the invention is an enzyme.

As used herein, the terms “peptide”, “polypeptide” and “protein” are interchangeable and refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides, polypeptides, and proteins described have modifications rendering them more stable while in the organism or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

As used herein, the terms “isolated polypeptide” refers to a protein that is essentially free from contaminating cellular components, such as carbohydrate, lipid, or other proteinaceous impurities associated with the nucleic acid in nature. Typically, a preparation of an isolated protein contains the protein in a highly purified form, e.g., at least about 80% pure, at least about 90% pure, at least about 95% pure, greater than 95% pure, or greater than 99% pure. In some embodiments, the isolated polypeptide is a synthesized polypeptide. Synthesis of protein is well known in the art and may be performed, for example, by heterologous expression in a transformed cell, such as exemplified herein.

The terms “homology” or “identity”, as used interchangeably herein, refer to sequence identity between two amino acid sequences or two nucleic acid sequences, with identity being a stricter comparison. The phrases “percent identity or homology” and “% identity or homology” refer to the percentage of sequence identity found in a comparison of two or more amino acid sequences or nucleic acid sequences. Two or more sequences can be anywhere from 0-100% identical, or any value there between. Identity can be determined by comparing a position in each sequence that can be aligned for purposes of comparison to a reference sequence. When a position in the compared sequence is occupied by the same nucleotide base or amino acid, then the molecules are identical at that position. A degree of identity of amino acid sequences is a function of the number of identical amino acids at positions shared by the amino acid sequences. A degree of identity between nucleic acid sequences is a function of the number of identical or matching nucleotides at positions shared by the nucleic acid sequences. A degree of homology of amino acid sequences is a function of the number of amino acids at positions shared by the polypeptide sequences.

The following is a non-limiting example for calculating homology or sequence identity between two sequences (the terms are used interchangeably herein). The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frame shift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

In some embodiments, % homology or identity as described herein are calculated or determined using the basic local alignment search tool (BLAST). In some embodiments, % homology or identity as described herein are calculated or determined using Blossum 62 scoring matrix.

According to some embodiments, there is provided a transgenic or transfected cell comprising: (a) the artificial DNA molecule of the invention; (b) the vector of the invention; (c) the polypeptide of the invention; or (d) any combination of (a) to (c).

As used herein, the term “transgenic cell” refers to any cell that has undergone human manipulation on the genomic or gene level. In some embodiments, the transgenic cell has had exogenous polynucleotide, such as an isolated DNA molecule as disclosed herein, introduced into it. In some embodiments, a transgenic cell comprises a cell that has an artificial vector introduced into it. In some embodiments, a transgenic cell is a cell which has undergone genome mutation or modification. In some embodiments, a transgenic cell is a cell that has undergone CRISPR genome editing. In some embodiments, a transgenic cell is a cell that has undergone targeted mutation of at least one base pair of its genome. In some embodiments, the exogenous polynucleotide (e.g., the isolated DNA molecule disclosed herein) or vector is stably integrated into the cell. In some embodiments, the transgenic cell expresses a polynucleotide of the invention. In some embodiments, the transgenic cell expresses a vector of the invention. In some embodiments, the transgenic cell expresses a protein of the invention. In some embodiments, the transgenic cell, is a cell that is devoid of a polynucleotide of the invention that has been transformed or genetically modified to include the polynucleotide of the invention. In some embodiments, CRISPR technology is used to modify the genome of the cell, as described herein.

In some embodiments, the cell is a unicellular organism, a cell of a multicellular organism, and a cell in a culture.

In some embodiments, a unicellular organism comprises a fungus or a bacterium.

In some embodiments, the fungus is a yeast cell.

In some embodiments, the cell is a prokaryote or a eukaryote cell.

In some embodiments, the artificial DNA molecule comprises a nucleic acid sequence codon optimized for expression (of the polypeptide of the invention) in the transgenic or transfected cell of the invention. In some embodiments, the additional nucleic acid sequence (e.g., encoding an enzyme, as disclosed herein), comprises a nucleic acid sequence codon optimized for expression (of the enzyme) in the transgenic or transfected cell of the invention.

Means and for determining codon optimized artificial DNA sequences and synthesizing thereof, are common and would be apparent to one ordinary skill in the art of molecular and cellular biology.

According to some embodiments, there is provided an extract, lysate, or homogenate derived from the transgenic or transfected cell of the invention, or any fraction thereof.

In some embodiments, the extract comprises dopamine.

Methods and/or means for extracting, lysing, homogenizing, fractionating, or any combination thereof, a cell or a culture of same, are common and would be apparent to one of ordinary skill in the art of cell biology and biochemistry. Non-limiting examples include, but are not limited to, pressure lysis (e.g., such as using a French press), enzymatic lysis, soluble-insoluble phase separation (such for obtaining a supernatant and a pellet), detergent-based lysis, solvent (e.g., polar, or nonpolar solvent), liquid chromatography mass spectrometry, or others.

In some embodiments, the transgenic plant, transgenic plant tissue or plant part, comprises: (a) the polynucleotide/artificial DNA molecule disclosed herein; (b) the vector disclosed herein; (c) the polypeptide of the invention; (d) the transgenic or transfected cell disclosed herein; or any combination thereof.

In some embodiments, the transgenic plant is a transfected and/or transformed plant.

In some embodiments, the transgenic plant comprises: a transgenic plant tissue or a plant part, any portion thereof, seed, tissue, organ thereof, or any combination thereof. In some embodiments, the transgenic plant comprises at least one transgenic plant cell as disclosed herein.

In some embodiments, the transgenic plant, transgenic plant tissue, or plant part consists of transgenic plant cells of the invention. In some embodiments, the transgenic plant, transgenic plant tissue, or plant part comprises at least: 20%, 25%, 30%, 35%, 40%, 45%, 50%, 55%, 60%, 65%, 70%, 75%, 80%, 85%, 90%, 95%, 97%, or 99% transgenic cells of the invention, or any value and range therebetween. Each possibility represents a separate embodiment of the invention. In some embodiments, the transgenic plant, transgenic plant tissue, or plant part comprises 20%-50%, 20%-60%, 20%-70%, 20%-80%, 20%-90%, or 20%-100% transgenic cells of the invention. Each possibility represents a separate embodiment of the invention.

According to some embodiments, there is provided a composition comprising: (a) the artificial DNA molecule of the invention; (b) the vector of the invention; (c) the polypeptide of the invention; (d) the transgenic or transfected cell of the invention; (e) the extract of the invention; or (f) any combination of (a) to (e), and an acceptable carrier.

As used herein, the term “carrier”, “excipient”, or “adjuvant” refers to any component of a composition, e.g., pharmaceutical or nutraceutical, that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier (e.g. carbomer, hydroxypropyl cellulose, sodium lauryl sulfate) as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers, and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers, and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

### General

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value. For example, a length of about 1,000 nanometers (nm) refers to a length of 1,000 nm+100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Materials and Methods

### Chemicals and Plant Material

All the analytical metabolites were >95% pure. Trp 1 and TAM 2 were purchased from Sigma-Aldrich (Rehovot, Israel); NMT 3, DMT 4, 5-HT 5, 5-MeO-TAM 8, 4-HT 14 and norbaeocystin 17 were purchased from Cayman Chemical (Ann Arbor, MI, USA); and 6-HT 11 was purchased from Arctom Scientific (Westlake Village, California, USA). All plants were acquired from a local nursery in Israel, propagated via cuttings and grown in a net house.

### LC-MS Chemical Analysis

Unless otherwise stated, 100 mg frozen powdered plant tissue was extracted with 300 L 80% methanol and 0.1% formic acid, sonicated for 30 min, agitated for 30 min and centrifuged at 14,000 g for 10 min. The supernatant was filtered through a 0.22 μm syringe filter. Samples were analyzed using an ultra-performance liquid chromatography (UPLC, Waters Acquity) with a diode array detector connected either to an Orbitrap IQ-X Tribrid MS or an Obitrap Exploris 240 MS (Thermo Scientific, Bremen, Germany). Chromatographic separation was performed on a 100 mm×2.1 mm i.d. (internal diameter), 1.7 μm UPLC BEH-T3 C18 column (Waters Acquity). The mobile phase consisted of 0.1% formic acid in acetonitrile:water (1:99, v/v; phase A) and 0.1% formic acid in acetonitrile (phase B). The flow rate was 0.3 mL min-1, and the column temperature was kept at 35° C. Two multistep methods were applied: In UPLC Method I, initial conditions were 0% B for 4 min, raised to 75% B until 12 min, raised to 100% B in 0.2 min, held at 100% B until 15 min, decreased to 0% B in 0.2 min, and held at 0% B until 18 min for re-equilibration of the system. In UPLC Method II conditions were 15% B for 0.5 min, raised to 30% B until 4 min, raised to 100% B in 0.2 min, held at 100% B until 7 min, decreased to 0% B in 0.2 min, and held at 0% B until 10 min for re-equilibration of the system.

Electrospray ionization (ESI) was used in positive ionization mode at m/z range of 70-1,000 Da. The source parameters on the Orbitrap MS systems were: sheath gas flow rate, auxiliary gas flow rate and sweep gas flow rate: 45, 10 and 1 arbitrary units, respectively; vaporizer temperature: 300° C.; ion transfer tube temperature: 320° C.; spray voltage: 3.5 kV. The instrument was operated in full MS1 with data dependent MS/MS (MS-dd-MS2). Data acquisition in full MS1 mode was 60,000 resolution, normalized automatic gain control target was standard and automatic maximum injection time. Data acquisition in dd-MS2 mode was with 15,000 resolution, standard normalized AGC target, automatic maximum injection time, isolation window of 1.5 m/z and stepped normalized collision energies of 15, 30 and 45. Identification of metabolites 1-5, 8, 10, 14 and 17 was according to analytical standards. Metabolites 15, 16, 18 and 19 were identified based on accurate mass, retention time, and MS/MS fragmentation spectra using a reference extract from P. cubensis. All the other metabolites produced in in vitro and/or in planta assays with specific enzymes were putatively identified based on LC-MS/MS as detailed herein. Some differences in fragmentation spectra, retention times and ion abundance were observed between instruments, therefore metabolites were compared in each run versus a standard and/or an extract analyzed under similar conditions.

### Absolute Quantification

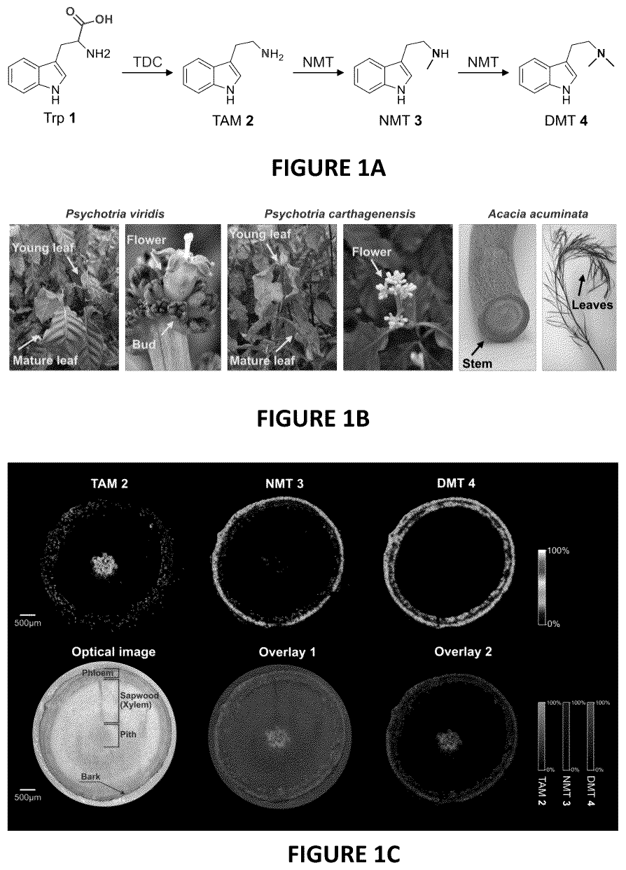

Fresh samples of young and mature leaves, buds, flowers, stems, and roots were collected from P. viridis and P. carthagenensis, while bark, sapwood, stems, leaves, and roots were collected from A. acuminata (FIG. 1B). Sapwood was manually separated from stems using a scalpel. For metabolite extraction, 100 mg of frozen powdered tissue was weighed in triplicate and extracted with 1 mL of 80% methanol containing 0.1% formic acid, as previously described. For absolute quantification of tertiary indolethylamines in N. benthamiana, four replicates of 100 mg frozen powder were extracted with 300 μL of 80% methanol and 0.1% formic acid. Quantification of Trp 1, TAM 2, NMT 3, DMT 4, and 5-MeO-DMT 10 was performed by external calibration using standards prepared in the same extraction solvent. Samples were injected at multiple dilutions to ensure signal linearity within the calibration range and analyzed in full MS1 mode as previously described. Absolute quantification of psilocin 16 and psilocybin 19 was performed in the laboratory of Prof. David Meiri using external calibration with an LC-MS method comparable to UPLC Method I, employing a similar column and mobile phase. Bufotenin 7 and 5-MeO-NMT 9 were semi-quantified using the calibration curves of psilocin 19 and 5-MeO-DMT 10, respectively assuming similar ionization efficiencies.

### MALDI Imaging

For localization of metabolites in A. acuminata stems, samples were cryo-sectioned (n=2 biological replicates), and matrix was sprayed using a TM sprayer (HTX Technologies, USA) with 2,5-dihydroxybenzoic acid (DHB; 40 mg mL⁻¹ in 70% containing 0.2% trifluoroacetic acid). The nozzle temperature was set at 70° C. and the DHB matrix solution was sprayed 16 passes over the tissue sections at a linear velocity of 120 cm min⁻¹ with a flow rate of 50 μL min 1. Sections were imaged with a Nikon DS-Ri2 microscope. MALDI imaging was performed using a 7 T Solarix FT-ICR (Fourier Transform Ion Cyclotron Resonance) mass spectrometer (Bruker Daltonics). Data acquisition and analysis were performed using flexImaging V4.1 (Bruker). The datasets were collected in positive ionization mode using lock mass calibration (DHB matrix peak: [3DHB+H-3H2O]⁺, m/z 409.055408 Da) at a frequency of 1 kHz and a laser power of 40%, with 200 laser shots per pixel and 50 μm step size. Each mass spectrum was recorded in the range of m/z 120-2,000 in broadband mode with a Time Domain for Acquisition of 1M, providing an estimated resolving power of 115,000 at m/z 400. The spectra were normalized to root-mean-square intensity and MALDI images were plotted at theoretical m/z±0.005% with pixel interpolation on.

### RNA Sequencing and Annotation

RNA was extracted from young and mature leaves, buds, flowers, stems, and roots of P. viridis and P. carthagenensis, and bark, sapwood, stems, leaves, and roots of A. acuminata using the Spectrum Plant total RNA kit (Sigma-Aldrich) or the RNeasy Plant Mini Kit (Qiagen). RNA integrity was checked using a TapeStation instrument. Paired-end Illumina libraries were prepared and sequenced on Illumina NovaSeq X Plus Sequencing System (PE 2×150, approximately 20 million reads per sample; Novogene Co., Ltd, China). For Iso-Seq sequencing of A. acuminata, libraries were prepared following bulk Iso-Seq SMRTbell Prep Kit 3.0 (Pacific Biosciences) using a pool of the five tissues. Sequencing was performed on PacBio Sequel II platform (Sheba Cancer Research Center, Sheba Medical Center, Ramat Gan, Israel) using SMRT Cell 8M and the Sequel II Sequencing Kit, according to the manufacturer's recommendations.

Raw Illumina reads from P. viridis and P. carthagenensis were quality-trimmed using Trimmomatic, as implemented in Trinity with default parameters. The processed reads were then used for de novo transcriptome assembly using Trinity. The assembled transcriptome was used as a reference for read alignment using bowtie2, and the resulting SAM files were converted to BAM format using Samtools. Transcript abundances were estimated using RSEM and normalized using DESeq2. Pairwise-Pearson correlation coefficients were calculated using WGCNA and visualized in Cytoscape.

For A. acuminata, transcriptome assembly was carried out using the SMRT Link IsoSeq3 workflow, part of the SMRT Tools v11 suite. First, cDNA primer sequences were removed with lima, using the ccs parameter set. Isoseq3 refine was then used with the require-polya switch to obtain full-length, non-concatemer (FLNC) reads. FLNC reads were clustered using isoseq3 cluster with the use-qvs switch enabled, generating high-confidence consensus transcripts. Unique transcripts were mapped to the GCA_019022655.1_ASM1902265v1 genome assembly, using pbmm2 align with the following settings: preset ISOSEQ and sort. This mapping allowed for the determination of transcript origin and potential isoforms associated with each gene locus. To annotate exon-intron structures and identify transcript isoforms per gene locus, the isoseq3 collapse tool was used. The do-not-collapse-extra-5exons switch was applied to preserve 5′ variation, as recommended in the official documentation. Raw Illumina reads from A. acuminata were quality-trimmed using Trim Galore!. The processed reads were aligned against the transcriptome assembly using STAR and the resulting BAM files were indexed and sorted using Samtools. Aligned read counts were obtained using featureCounts and normalized using DESeq2. For all three transcriptomes, gene functional annotation was performed for the predicted transcripts as described in Berman et al., (Mol Plant 2024, 17:1129-1150) with additional analyses. Specifically, subcellular localization was predicted using TargetP 2.0 and DeepLoc 2.0 and enzymatic functions were predicted with ECPred.

### Phylogenetic Analysis of MTs

Proteins in the phylogenetically analysis was based on functionally tested OMTs and NMTs. The Maximum Likelihood tree was constructed with 1,000 bootstrap tests based on a MUSCLE multiple alignment using the MEGA11 software.

Recombinant Protein Expression in E. coli

PvTDC1, PvTDC2, PvNMT1, PvMT2, AaNMT1 and AaMT2-AaMT6 coding sequences were amplified by PCR from cDNA of P. viridis or A. acuminata AaNMT7 was synthesized by the company Twist Biosciences. Genes were individually cloned into the pET28b vector digested with EcoRI using the ClonExpress II one step cloning kit (Vazyme, Germany). AtCOMT and AtCOMTA160G coding sequences were codon optimized for E. coli expression and synthesized and cloned into pET28a vector by the company Twist Biosciences. All constructs were sequenced and expressed in E. coli BL21 (DE3) pLysS competent cells. Bacterial starters were grown overnight in LB medium with kanamycin (50 μg/ml) at 37° C., diluted (1:100) in fresh LB with kanamycin (50 μg/ml), and re-incubated at 37° C. When cultures reached A600=0.6, protein expression was induced with 500 μM of isopropyl-1-thio-β-d-galactopyranoside (IPTG) overnight at 16° C. Cultures were centrifuged at 4,300 rpm for 10 min and the cell pellets were lysed by sonication in 50 mM Tris-HCl (pH 8.0), 0.5 mM phenylmethylsulfonyl fluoride (PMSF, Sigma Aldrich) solution in isopropanol, 10% glycerol and protease inhibitor cocktail (Sigma Aldrich), 0.1 mg mL⁻¹ lysozyme (Sigma Aldrich) and 0.01% benzonase nuclease (Sigma Aldrich). The whole-cell extract (lysate) was either kept for functional activity or used for protein purification. Purification of hexahistidine-tagged proteins was performed using Capturem™ His-tagged purification 24-well plate (Takara Bio., USA) according to the manufacturer's protocol. The proteins were eluted with 2 mL 500 mM imidazole (Fluka) in buffer containing 20 mM NaH₂PO₄ (pH 7.6) and 500 mM NaCl. Desalting was performed by repeated centrifugation at 4° C., 4,300 r.p.m. using Amicon Ultra 10 K column (Merck Millipore, Tullagreen, Carrigtwohill, Ireland) in Tris-HCl (pH 8.0). Protein purity was assessed by gel electrophoresis using a 10% (w/v) polyacrylamide gel. Protein purification and enzymatic assays were performed consecutively on the same day.

### TDC and NMT Enzyme Assays

TDC enzyme assays were performed in 50 μL with 100 mM Tris-HCl buffer (pH 8.0), 1 mM PLP, 2 mM Trp 1 and 20 μL of the lysate enzyme solution or 2 μL of the purified enzymes. NMT and OMT enzyme assays were performed in 50 μL with 100 mM Tris-HCl buffer (pH 8.0), 1 mM SAM, 1 mM of indolethylamine substrates and 20-30 μL of the lysate enzyme solution. To examine the activity of AtCOMT and AtCOMTA160G with bufotenin 7, an extract prepared from N. benthamiana leaves co-expressing AroG, PvTDC2, OsCYP71P1, and RmNMT was employed, which supplied bufotenin 7 for the enzyme assays. Enzyme assays with mixture of lysate expressing either PvTDC1, PvTDC2 or PvNMT1 were similarly performed with 1 mM PLP, 1 mM SAM, 2 mM Trp 1 as the only substrate, and 20 μL of each lysate enzyme solution. The assay mixtures were incubated at 30° C. for 2 h, after which the reactions were terminated by the addition of 70 μL ice-cold methanol with 0.1% formic acid, vortexing and centrifugation at 15,000 g for 10 min. The supernatant was filtered and analyzed by LC-MS.

Co-Expression of PvTDC2 and PvNMT1 in E. coli and In Vivo Assays

PvTDC2 and PvNMT1 were cloned simultaneously into pACYCDuet-1 vector (Merck-Novagen) by restriction free (RF) cloning. The construct was expressed in E. coli BL21 (DE3) competent cells. Bacterial starters were grown overnight in LB medium containing chloramphenicol (25 μg/ml) at 37° C., diluted 1:100 in fresh LB containing 25 μg/ml chloramphenicol, and re-incubated at 37° C. until A600=2. Protein expression was induced with 200 μM of isopropyl-1-thio-β-d-galactopyranoside (IPTG) and cultures were grown for 24 h at 30° C. Cultures were centrifuged at 4,300 rpm for 10 min and 100 μL of the supernatant were extracted with an equal volume of ice-cold methanol with 0.1% formic acid, followed by vortexing and centrifugation at 15,000 g for 10 min. The supernatant was filtered and analyzed by LC-MS using UPLC Method II.

Transient Expression of Genes in N. benthamiana

PvTDC1, PvTDC2, PvNMT1, and AaNMT1 coding sequences were individually cloned in pAlpha2-NPT II-Ubq10-CCD-Ter10 vector digested with ApaI using ClonExpress II One Step Cloning kit (Vazyme). RmNMT, PsiH, PsiK, PsiM, OsCYP71P1, AtCOMT, AtCOMT-Mut1, RebF, RebH, PyrH and SttH coding sequences were codon optimized for expression in Nicotiana tabacum and synthesized by Twist Biosciences. All but SttH were similarly cloned to the same vector. SttH was cloned into pDBG3a2 vector with 35s promoter and terminator sequences using the GoldenBraid cloning system. All plasmids were sequenced and transformed into Agrobacterium tumefaciens strain GV3101 by electroporation. A. tumefaciens harboring the overexpression constructs were grown overnight at 28° C. in Luria-Bertani (LB) medium in the presence of kanamycin and gentamycin (50 μg/ml each). For expression of the mutated AroG (AroG209 with the specific point mutation F209A) the inventors used GV3101 (J Exp Bot 2013, 64:4441-4452). AroG stocks were grown in the presence of rifampicin (25 μg/ml), spectinomycin (100 μg/ml) and gentamycin (50 μg/ml). Following overnight incubation, bacterial cells were collected by centrifugation, washed and resuspended in infiltration buffer (2 mM Na₃PO₄, 50 mM MES, 10 mM MgCl₂, 0.5% w/v glucose, and 100 μM acetosyringone) to OD600=0.5. Individual and/or equal volumes of A. tumefaciens suspension with different expression vectors were combined to obtain the desired gene combinations. The solutions were infiltrated into 4- or 5-week-old N. benthamiana leaves from the abaxial side using a 1-mL needleless syringe. Leaves were collected for metabolite analysis three to five days from infiltration. Leaf samples were flash frozen and extracted as previously described with 300 μL 80% methanol with 0.1% formic acid and analyzed via LC-MS.

### Modeling and Site Directed Mutations of AtCOMT

The structure of AtCOMT was modeled with AlphaFold 3, using the sequence of AtCOMT and the smiles annotations of SAM cofactor, 5-HT 5 and bufotenine 7. The model was visualized using PyMol software and the A160G mutation in the vicinity to the additional methyl groups of bufotenine 7 relative to serotonin 5 was designed rationally.

### Data Availability

All NGS raw sequencing data will be deposited in ENA following acceptance of the manuscript. The sequences of the genes coding for the active enzymes elucidated in this study (PvTDC1, PvTDC2, PvNMT1, AaNMT1 and AaNMT7) will be deposited in NCBI GenBank following acceptance of the manuscript. The protein databases used in this study are Uniprot Swiss-Prot release-2022_02, Solanum lycopersicum UP000004994_4081, Oryza sativa UP000059680_39947, and Spinacia oleracea UP000054095_3562, and from NCBI A. thaliana GCF_000001735.4 TAIR10.1, and Triticum aestivum GCF_018294505.1 IWGSC_CS_RefSeq_v2.1. The Pfam database was Pfam-A.hmm release 34.0.

### Example 1

Psychotria viridis and Acacia acuminata Produce DMT

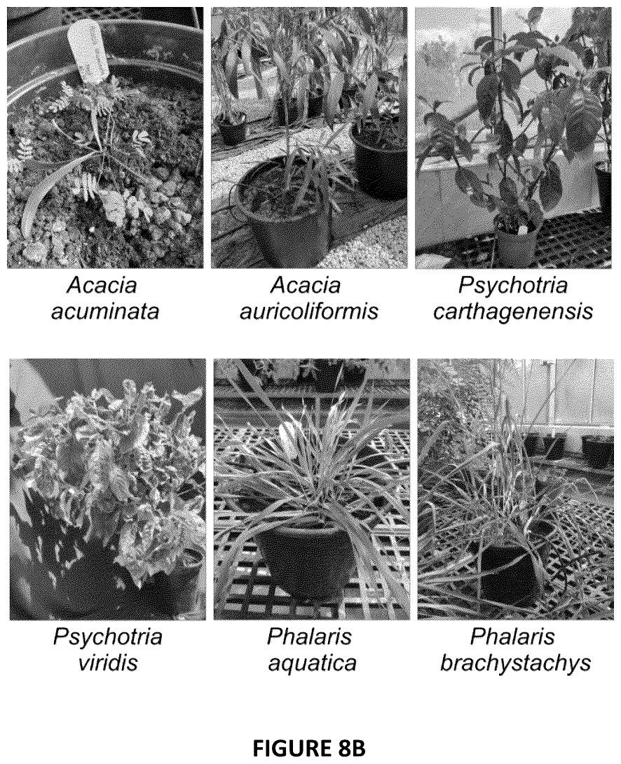

Reports suggest that a wide variety of plant species across multiple families accumulate DMT 4. To select plants for elucidating candidate biosynthetic genes, the inventors first screened for NMT 3 and DMT 4 in several Acacia, Phyllodium, Psychotria, and Phalaris plant species and tissues, using high-resolution liquid chromatography-tandem mass spectrometry (LC-MS/MS) (FIG. 8). Among these, the inventors further focused discovery on Psychotria viridis (P. viridis), a Central American plant from the Rubiaceae family known for its role in ayahuasca brews, and Acacia acuminata (A. acuminata), an Australian species from the Fabaceae family. The inventors also included Psychotria carthagenensis (P. carthagenensis), a close taxonomic relative of P. viridis that does not accumulate DMT 4 (FIG. 8).

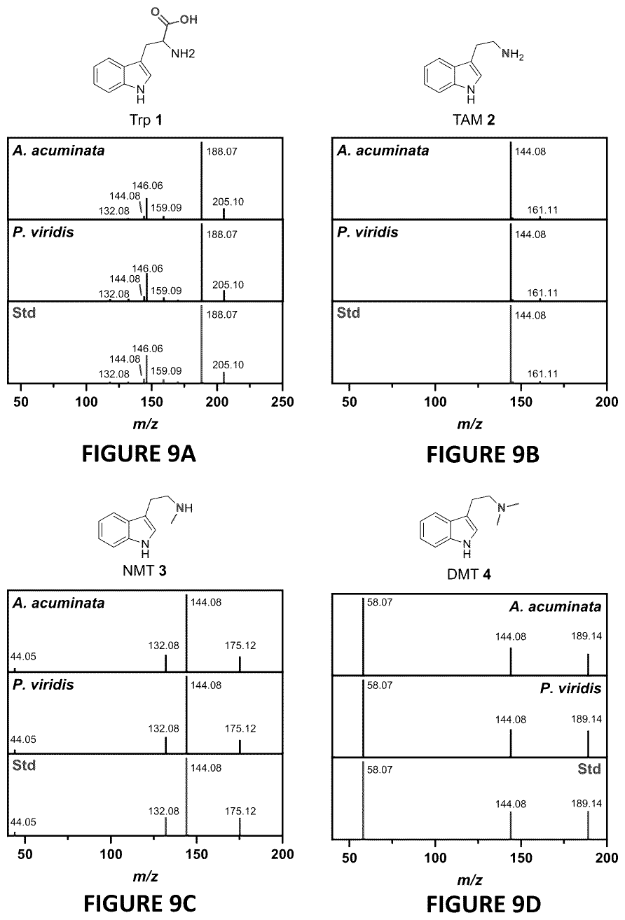

The inventors identified all pathway intermediates across different plant tissues (molecules 1-4, FIGS. 1A-1B and 9). In P. viridis, DMT 4 accumulated in all aerial parts, with the highest levels in young tissues such as leaves and flower buds (1.55±0.51% per fresh weight) but was absent in the roots. In contrast, P. carthagenensis showed no significant accumulation of indolethylamines (FIG. 1B). In A. acuminata, DMT 4 was present in all tissues, but most abundantly in the bark (0.77±0.24% per fresh weight; FIG. 1B).

Further analysis of A. acuminata stem samples using matrix-assisted laser desorption/ionization-mass spectrometry imaging (MALDI-MSI) revealed distinct localization patterns for pathway intermediates (FIG. 1C). TAM 2 was most abundant in the pith and phloem, while NMT 3 was present in the pith, highest accumulation was observed in the outer phloem layers and bark. DMT 4 was absent from the pith and sapwood, accumulating predominantly in the phloem and bark regions. This differential accumulation of intermediates across tissues suggests a distinct spatial expression pattern of enzymes involved in the biosynthetic pathway.

### Example 2

### Elucidation of the Complete DMT Pathway in Two Plants

To identify the biosynthetic enzymes active in this pathway the inventors obtained RNAseq data of different tissues from the three plants. Based on the predicted biosynthetic pathway (FIG. 1A), the inventors searched for candidate tryptophan decarboxylase (TDC) and N-methyltransferase (NMT) enzymes in P. viridis, prioritizing candidates based on their relative expression levels across tissues, protein size, presence of protein family (Pfam) motifs, and similarity to functionally characterized enzymes from other plants. The inventors identified two TDC candidates (PvTDC1 and PvTDC2), each with several isoforms coding for similar proteins.

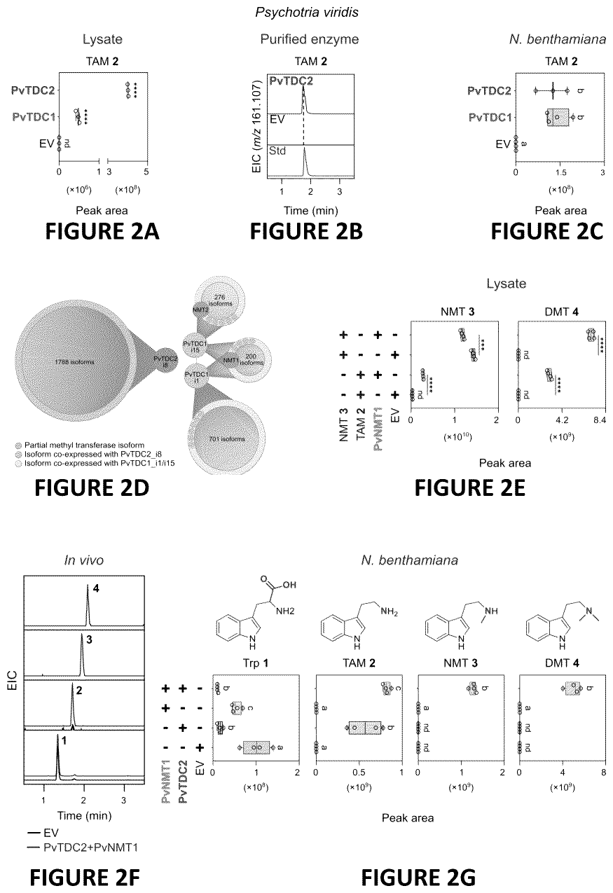

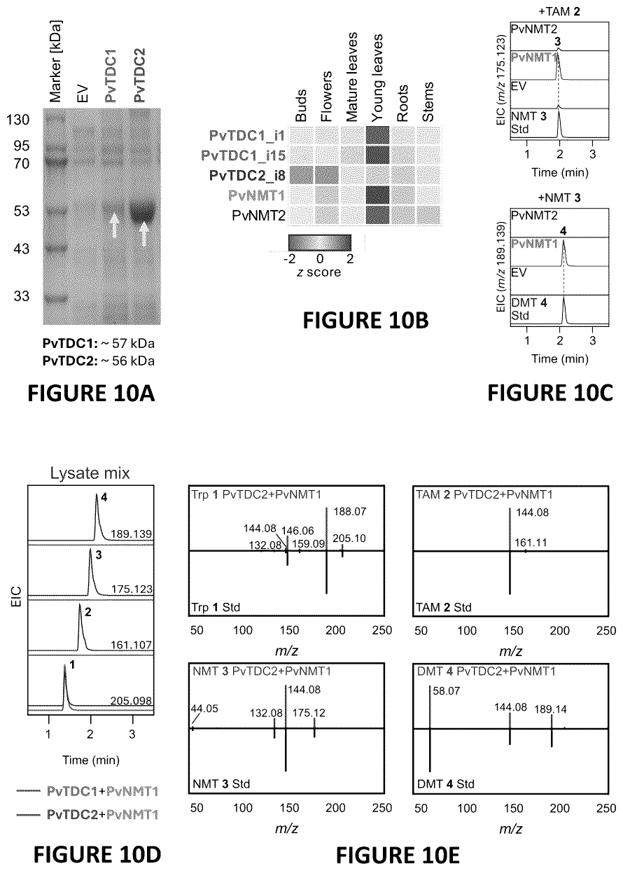

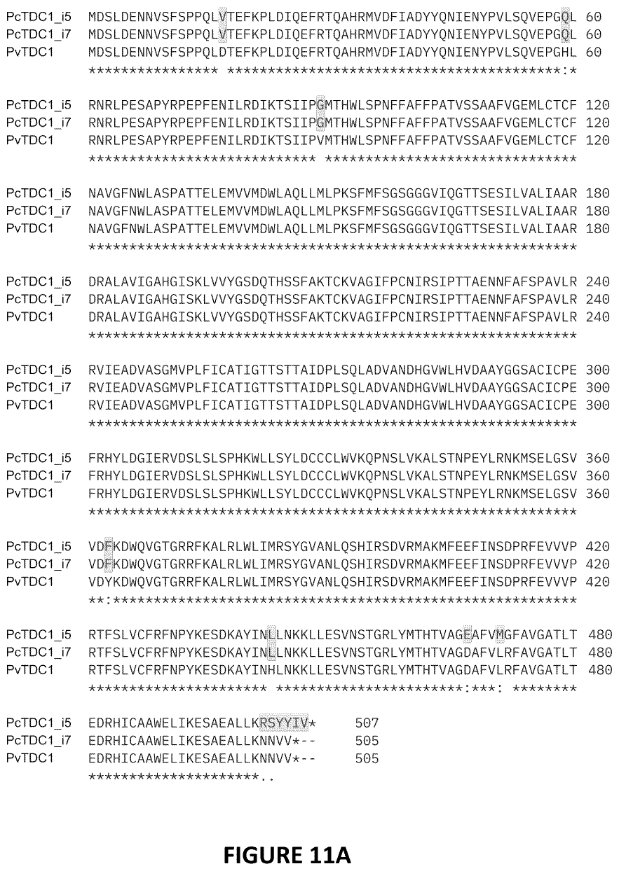

The inventors recombinantly expressed both candidates individually in Escherichia coli (E. coli) and screened for activity using lysate supplemented with pyridoxal 5′-phosphate (PLP) and Trp 1 as the substrate. Both enzymes produced TAM 2, though PvTDC2 exhibited peak areas nearly a thousand-fold higher than those of PvTDC1 (FIG. 2A). Purification of both enzymes revealed only a faint band for PvTDC1 on gel electrophoresis, suggesting poor in vitro expression or protein instability (FIG. 10A). Enzyme assays with purified PvTDC2 confirmed its catalytic activity (FIG. 2B). To further evaluate enzymatic activity in planta, the inventors transiently expressed PvTDC1 and PvTDC2 individually in N. benthamiana leaves via agroinfiltration. Both enzymes exhibited comparable activity, converting endogenous Trp 1 into TAM 2, without substrate supplementation (FIG. 2C). These results confirmed that both PvTDC1 and PvTDC2 function as active TDCs in P. viridis.

The inventors next used PvTDC1_i1, PvTDC1_i15, and PvTDC2_18 isoforms as baits for co-expression analysis since they were highly enriched in young leaves and buds but not expressed in roots (FIG. 9B). This analysis identified several genes annotated as methyltransferases, but only two sequences (PvNMT1 and PvNMT2 co-expressing with PvTDC1) corresponded to proteins of the expected size (FIG. 2D). Both candidates displayed the highest expression in young leaves and low expression in roots (FIG. 10B).

In vitro enzyme assays using E. coli lysate expressing each enzyme individually, with S-adenosyl-L-methionine (SAM) as the methyl donor and either TAM 2 or NMT 3 as substrates, showed that PvNMT1 catalyzed both N-methylation steps, producing NMT 3 and DMT 4 while PvNMT2 was inactive with both substrates (FIGS. 2E and 10C). Next, the inventors conducted enzyme assays by combining lysate expressing either PvTDC1, PvTDC2 or PvNMT1, using Trp 1 as the substrate in the presence of PLP and SAM. The combination of PvTDC2 and PvNMT1 resulted in the production of all pathway intermediates (FIG. 10D). Finally, by co-expressing PvTDC2 and PvNMT1 in the same cells, the inventors successfully produced DMT 4 and all pathway intermediates in vivo without the need for substrate or intermediate supplementation (FIGS. 2F and 10E).

To evaluate in planta DMT 4 production, the inventors agroinfiltrated PvNMT1 with and without PvTDC2 in N. benthamiana. Expression of PvTDC2 resulted in the accumulation of large quantities of TAM 2 at the expense of Trp 1 (FIG. 2G). Neither NMT 3 nor DMT 4 were detected, suggesting that N. benthamiana lacks an endogenous NMT enzyme with similar activity. Co-expression of PvNMT1 with PvTDC2 led to the de novo production of NMT 3 and DMT 4 (FIG. 2G).

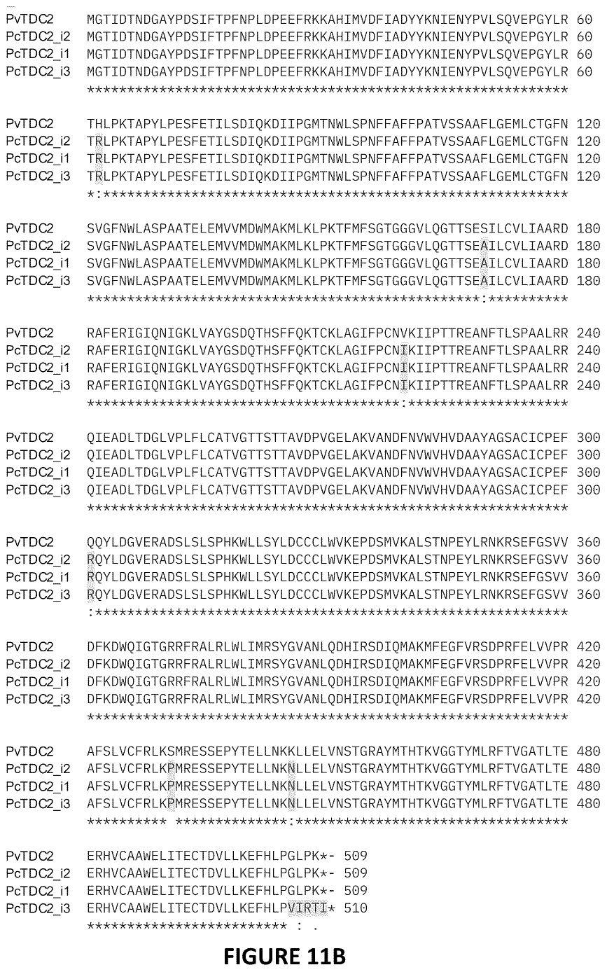

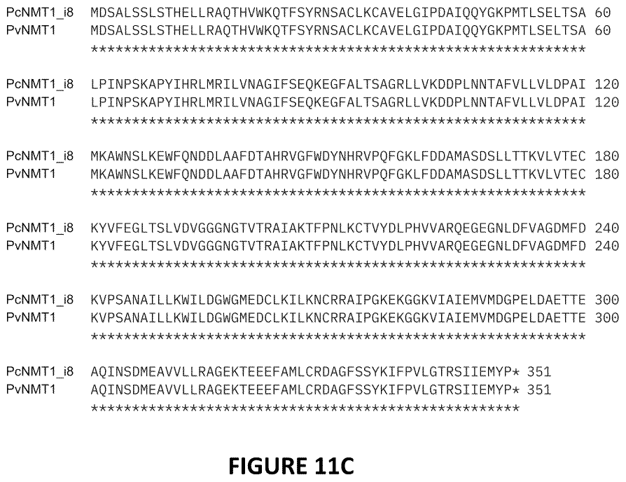

Notably, the inventors identified P. carthagenensis isoforms homologous to the active enzymes in P. viridis, namely PcTDCI, PcTDC2, and PcNMTI, which exhibit high amino acid sequence similarity to their P. viridis counterparts (FIG. 11). However, while PcTDC2 was expressed in flowers and mature leaves, PcTDCI and PcNMTI displayed consistently low expression across all tissues, likely accounting for the absence of DMT 4 and its intermediates in P. carthagenensis (FIG. 1B).

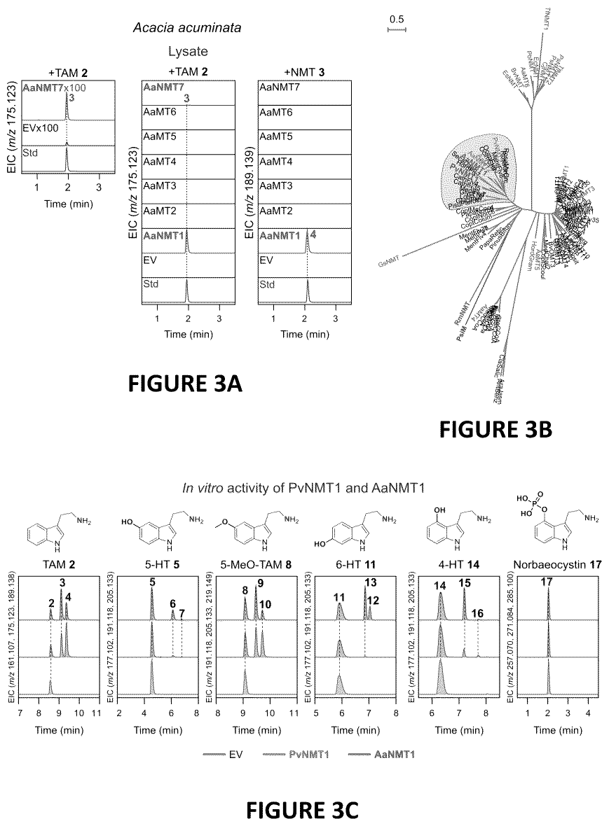

The inventors next searched for more NMT candidate genes in A. acuminata. The inventors identified seven genes (AaNMT1-AaNMT7) which were highly expressed in various tissues, with the highest expression observed in bark. Each candidate was recombinantly expressed and tested for activity using lysate, as previously described. Among the seven NMT candidates, AaNMT1 catalyzed both N-methylation steps, producing NMT 3 and DMT 4, similar to PvNMT1 (FIG. 3A). AaNMT7, which shares 84.6% protein sequence similarity with AaNMT1, exhibited lower activity and only catalyzed the first N-methylation step, converting TAM 2 to NMT 3 (FIG. 3A). Interestingly, a phylogenetic analysis of functionally characterized O-methyltransferases (OMTs) and NMTs grouped all three identified NMTs from this study within the clade of flavonoid-type OMTs and not with other functionally characterized NMTs (FIG. 3B).

### Example 3

### PvNMT1 and AaNMT1 Exhibit Promiscuous In Vitro Activity on Multiple Indolethylamine Derivatives

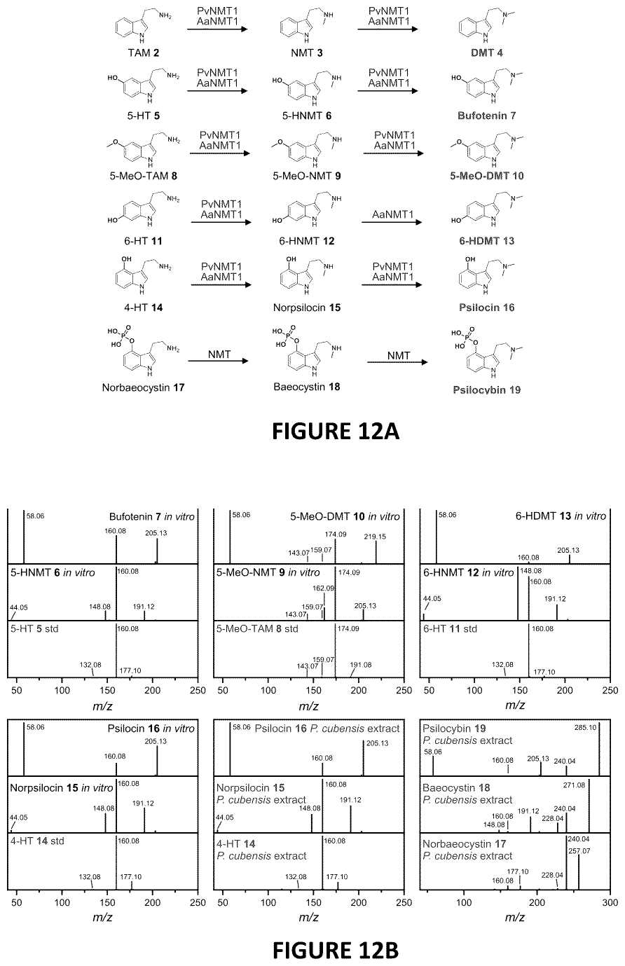

To test whether the identified plant NMTs could catalyze the formation of tertiary amines, similar to the recently identified NMT from the cane toad (Rhinella marina RmNMT, OQ557631), the inventors performed in vitro enzyme assays using lysate expressing either PvNMT1 or AaNMT1 with various amine substrates and SAM as the methyl donor. The inventors found that both enzymes displayed promiscuous activity with most tested compounds. Specifically, serotonin (5-HT 5) was converted to N-methyl-serotonin (5-HNMT 6) and bufotenin 7; 5-methoxytryptamine (5-MeO-TAM 8) was N-methylated to 5-methoxy-N-methyl-tryptamine (5-MeO-NMT 9) and 5-methoxy-N,N-dimethyltryptamine (5-MeO-DMT 10); 6-hydroxy-tryptamine (6-HT 11) was N-methylated to 6-hydroxy-N-methyl-tryptamine (6-HNMT 12) and 6-hydroxy-N,N-dimethyltryptamine (6-HDMT 13); and 4-hydroxy-tryptamine (4-HT 14) gave rise to norpsilocin 15 and psilocin 16 (FIGS. 3C and 12). However, neither PvNMT1 nor AaNMT1 catalyzed the N-methylation of norbacocystin 17 to baeocystin 18 or psilocybin 19 (FIG. 3C). Interestingly, PvNMT1 showed better relative activities for the production of DMT 4 and 5-MeO-TAM 8, while AaNMT1 was more active for the N-methylation of hydroxylated tryptamines. These findings highlight the potential of plant NMTs for the heterologous biosynthesis of structurally related indolethylamines with therapeutic potential.

### Example 4

### In Planta Reconstruction of Psychedelic Biosynthetic Pathways

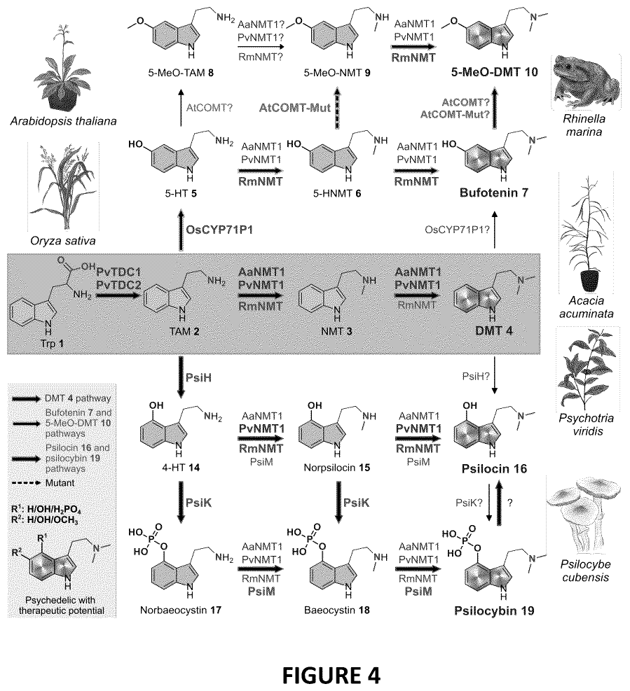

The next sought to leverage the endogenous pool of Trp 1 in N. benthamiana, to reconstruct complete biosynthetic pathways of psychedelics derived from plants, toads and magic mushrooms. The inventors applied a metabolic engineering approach, integrating the herein discovered enzymes from P. viridis and A. acuminata with previously characterized genes (FIG. 4). For the psilocybin pathway, the inventors utilized the monooxygenase (PsiH, MF000993) and kinase (PsiK, KY984099) previously elucidated from Psilocybe cubensis (P. cubensis), hypothesizing that psilocybin 19 could be produced from DMT 4 or related derivatives (FIG. 4). In contrast, the biosynthetic pathway of 5-MeO-DMT 10 in toads remains incomplete, with RmNMT being the only identified enzyme thus far. To enable the biosynthesis of 5-MeO-DMT 10 and bufotenin 7, the inventors used OsCYP71P1 (AK071599) from Oryza sativa (rice), which catalyzes the 5-hydroxylation of TAM 2 to 5-HT 5, and the caffeic acid OMT (AtCOMT, At5g54160) from Arabidopsis thaliana (A. thaliana), which has been suggested to O-methylate 5-HT 5 to 5-MeO-TAM 8. The inventors hypothesized that AtCOMT may also O-methylate bufotenin 7 (FIG. 4).

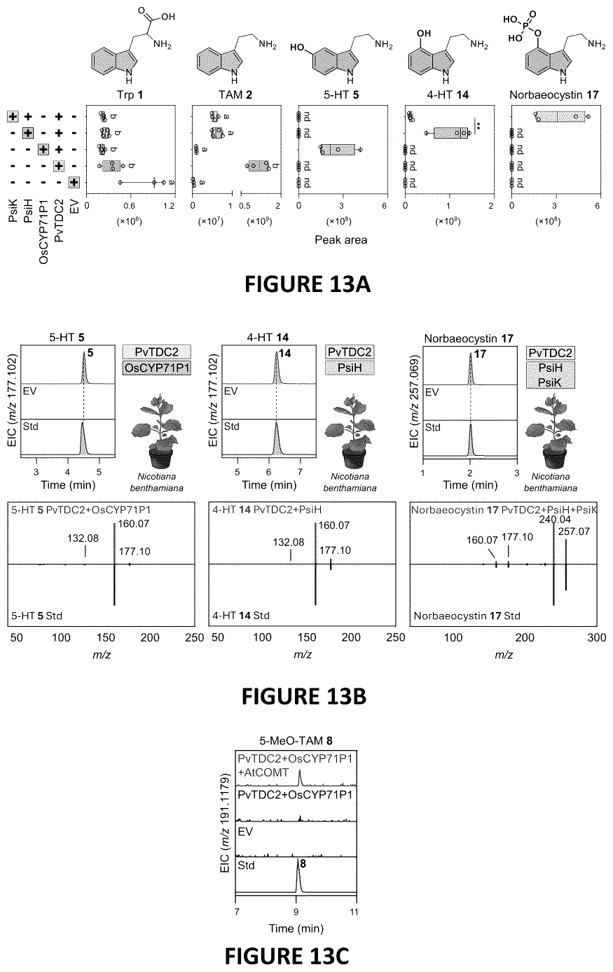

Agroinfiltration of PvTDC2 with either OsCYP71P1, PsiH, or PsiH and PsiK to N. benthamiana leaves, led to the accumulation of 5-HT 5, 4-HT 14 and norbaeocystin 17, respectively (FIGS. 13A-13B). The expression of either PsiH or OsCYP71P1 resulted in a pronounced depletion of TAM 2, suggesting high catalytic efficiency in these pathways. However, no secondary or tertiary products were detected in any of the treatments, indicating that N. benthamiana lacks endogenous NMTs capable of catalyzing these reactions. Further co-expression of PvTDC2, OsCYP71P1 and AtCOMT showed a low abundance peak of 5-MeO-TAM 8, suggesting low catalytic activity of AtCOMT with 5-HT 5 in N. benthamiana (FIG. 13C).

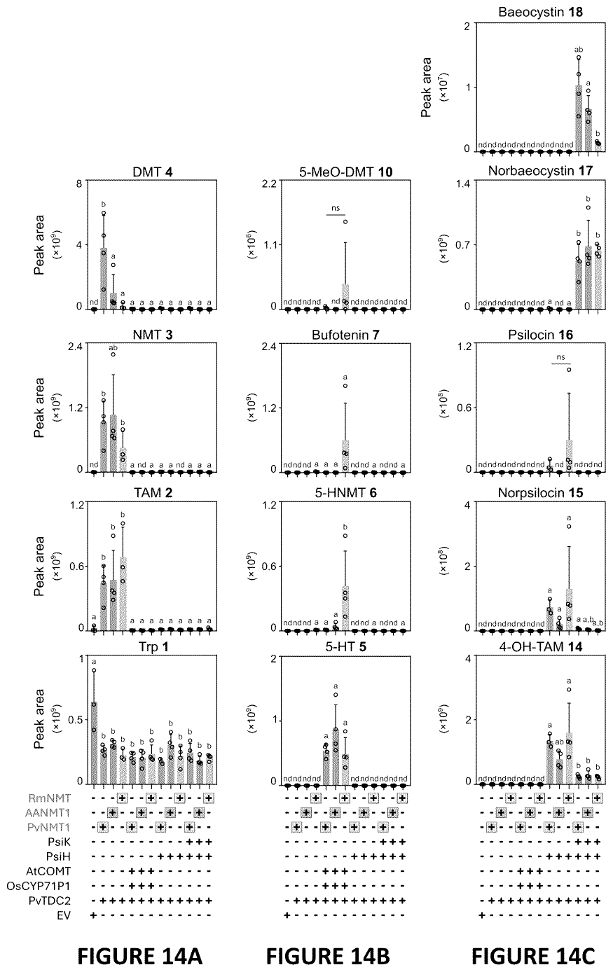

To complete the biosynthetic pathways for the production of classical psychedelics, the inventors repeated the experiments, this time co-expressing either PvNMT1, AaNMT1, or RmNMT1 in combination with each of the previous treatments (FIG. 4). Co-expression of PvTDC2 with any of the NMTs resulted in the production of DMT 4, with PvNMT1 yielding the highest concentrations (FIG. 14A). However, co-infiltration with PsiH or OsCYP71P1 significantly reduced TAM 2 availability, leading to markedly lower levels of DMT 4 in these treatments (FIG. 14A). This suggests that the hydroxylases exhibit higher efficiency with TAM 2 than any of the NMTs, thereby depleting the substrate pool and limiting DMT 4 biosynthesis.

Co-expression of PvTDC2, OsCYP71P1, AtCOMT, and RmNMT successfully resulted in large concentrations of 5-HT 5, 5-HNMT 6, and bufotenin 7. However, only low-abundance peaks of 5-MeO-DMT 10 were detected, suggesting that AtCOMT exhibits poor catalytic activity also with bufotenin 7 (FIGS. 4 and 14B). PvNMT1 and AaNMT1 were considerably less active on this pathway compared to RmNMT (FIG. 14B). Co-expression of PvTDC2, PsiH, and either PvNMT1 or RmNMT led to the accumulation of 4-HT 14, norpsilocin 15, and psilocin 16, whereas AaNMT1 failed to produce psilocin 16 (FIG. 14C). The phosphorylated intermediates norbaeocystin 17 and baeocystin 15 were detected only in treatments that included co-infiltration with PsiK (FIG. 14C); however, no psilocybin 19 was observed. Given the lack of in vitro activity of norbaeocystin 17 with either PvNMT1 or AaNMT1 (FIG. 3C), the production of baeocystin 18 likely occurs via phosphorylation of norpsilocin 15 by PsiK (FIG. 4). Co-expression of PsiK led also to a significant reduction in all the non-phosphorylated intermediates from mushrooms (metabolites 14-16, FIG. 14C).

### Example 5

### Engineering Approaches to Increase the Accumulation of Indolethylamines

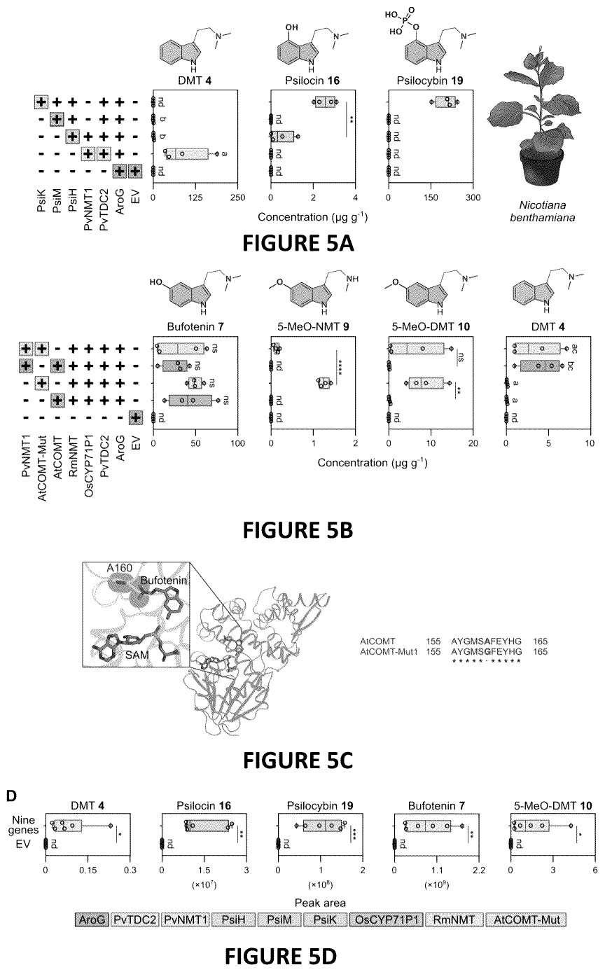

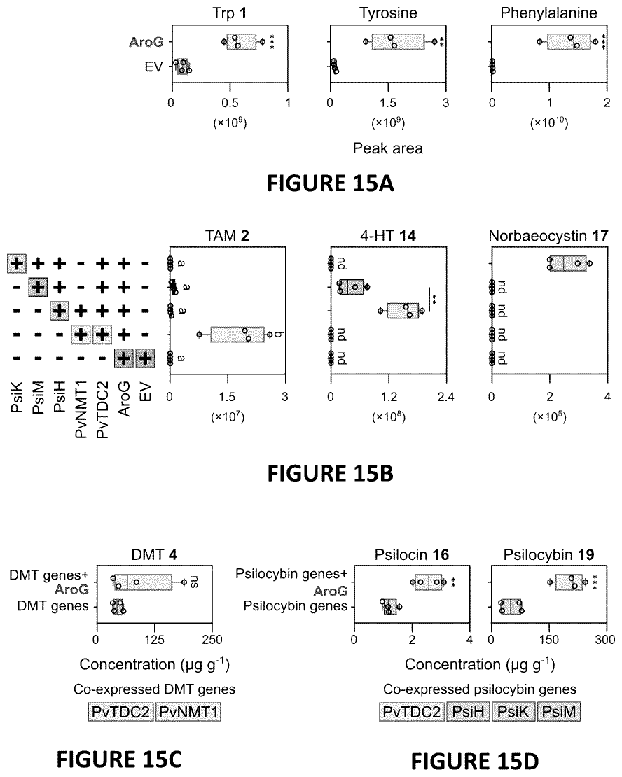

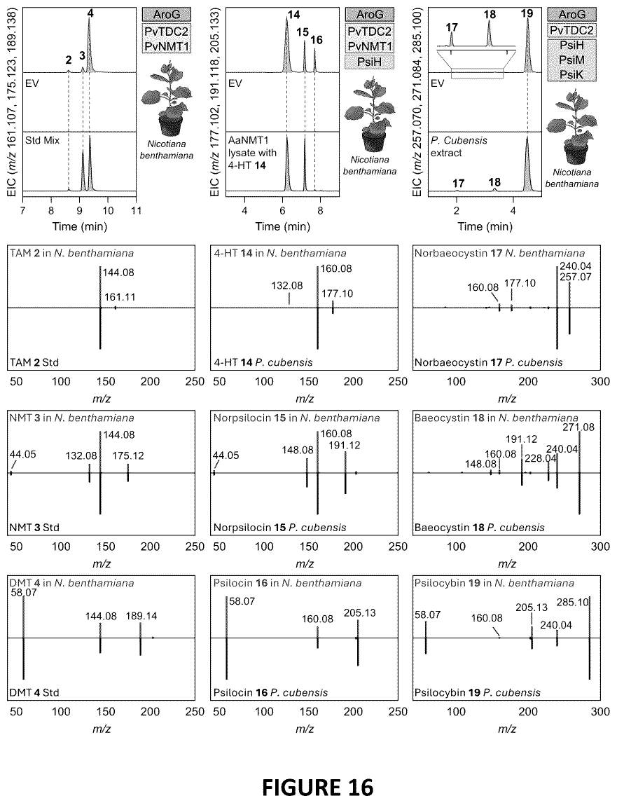

To enhance Trp 1 availability in N. benthamiana, the inventors expressed AroG, a feedback-insensitive mutant of bacterial deoxy-D-arabino-heptulosonate 7-phosphate synthase (AroG209 harboring a specific point mutation at F209A), whose overexpression has been shown to increase aromatic amino acid levels and their derived metabolites (FIG. 15A). Co-expression of AroG with PvTDC2 and PvNMT1 led to the accumulation of DMT 4 at an average concentration of 89 μg·g⁻¹ fresh weight, one week post-agroinfiltration (FIGS. 5A and 15B-15C). To assess psilocin 16 production, the inventors co-expressed AroG, PvTDC2, and PsiH with either PvNMT1 or PsiM (KY984100), the NMT previously identified in P. cubensis. PsiM exhibited no activity on non-phosphorylated substrates (FIG. 5A), whereas expression of PvNMT1 produced on average 0.64 μg g⁻¹ psilocin 16 (FIG. 5A). Further co-expression of AroG, PvTDC2, PsiH, PsiM, and PsiK enabled the biosynthesis of norbacocystin 17, baeocystin 18, and psilocybin 19 (205.4 ugg-1 on average), alongside psilocin 16 (2.6 μg·g⁻¹ on average), which likely accumulated through spontaneous dephosphorylation or endogenous phosphatase activity in N. benthamiana (FIGS. 4, 5A, 15B, and 16, and table S5 of the appendix file). Notably, the inclusion of AroG led to a fourfold increase in psilocybin 19 accumulation (FIG. 15D, and table S5 of the appendix file).

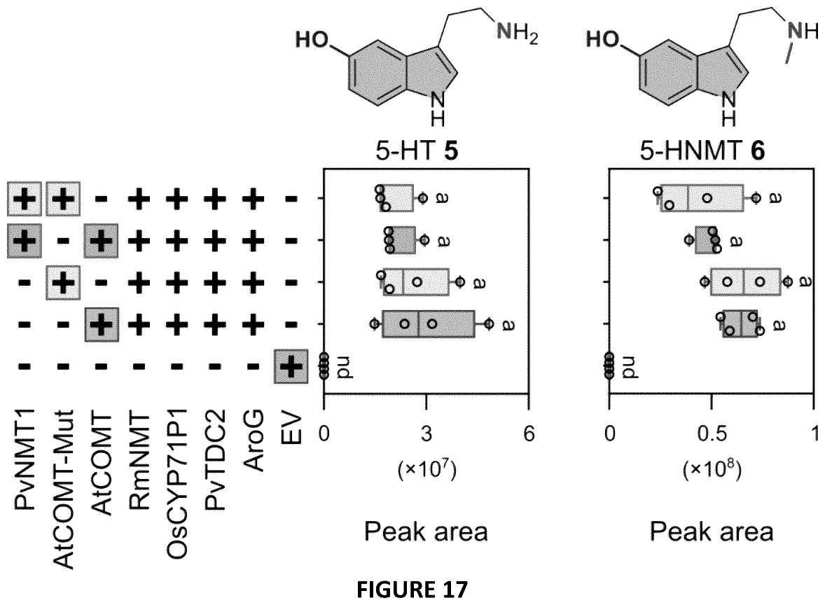

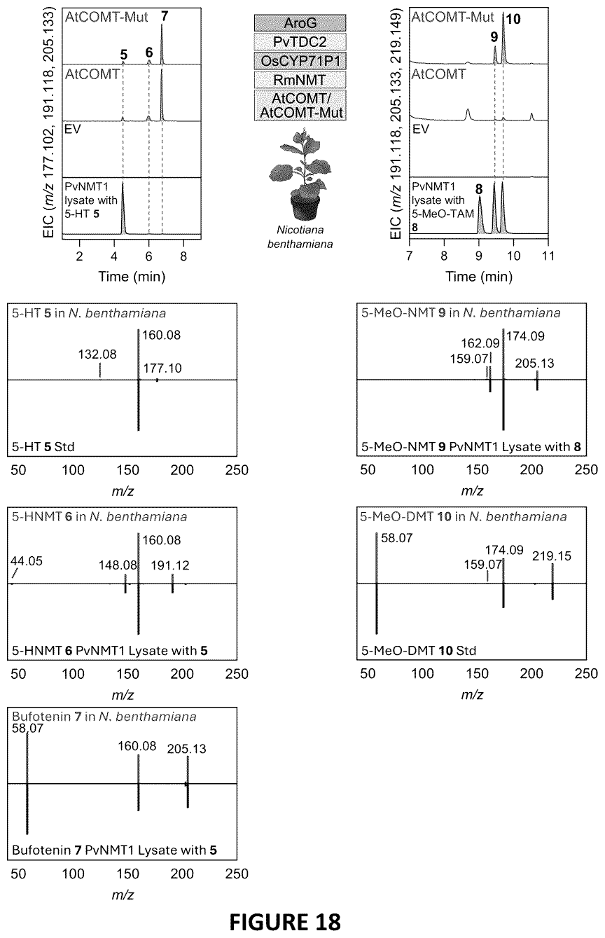

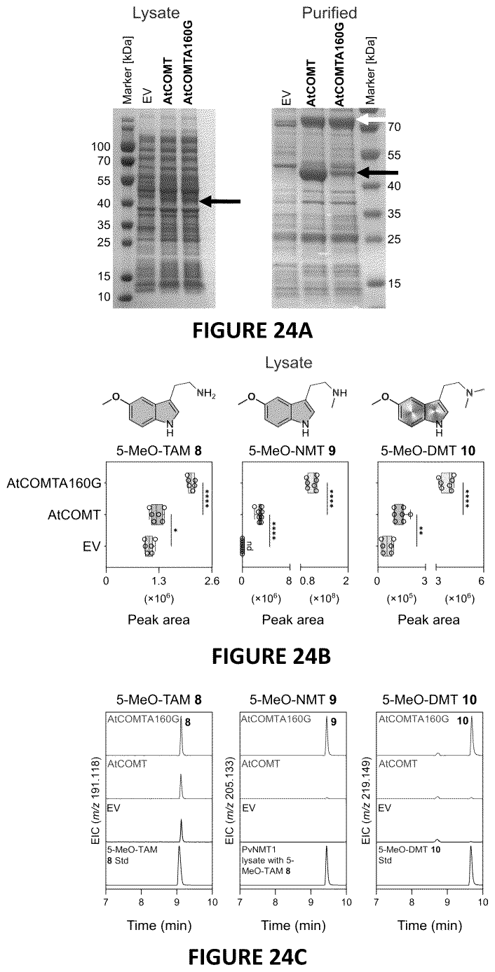

To increase the production of 5-MeO-DMT 10, the inventors hypothesized that targeted mutations in AtCOMT, designed to enlarge the amine-binding pocket, could shift the enzyme's substrate preference from 5-HT 5 to bufotenin 7. Using AlphaFold3, the inventors modeled AtCOMT in complex with SAM cofactor and either 5-HT 5 or bufotenin 7 as substrates, and rationally designed a mutation A160G (AtCOMT-Mut1 (SEQ ID Nos: 13-14)) to accommodate the methyl groups of bufotenin 7 in the active site (FIG. 5C). Co-expression of AroG, PvTDC2, OsCYP71P1, RmNMT, and AtCOMT-Mut1 (SEQ ID Nos: 13-14) in N. benthamiana significantly increased levels of 5-MeO-DMT 10 (0.2 vs. 8.5 μg·g⁻¹ on average of compound 10; FIG. 5B) compared to the wild-type AtCOMT. Interestingly, co-expression of AtCOMT-Mut1 additionally led to the accumulation of 5-MeO-NMT 9 which was not observed with the wild type AtCOMT (FIG. 5B). No significant differences were observed in the accumulation of bufotenin 7 (42.8 μg·g⁻¹ with AtCOMT), 5-HT 5, or 5-HNMT 6 (FIGS. 5B, 17 and 18). To evaluate substrate specificities, AtCOMT and AtCOMTA160G were expressed in E. coli and attempted to purify the proteins. Surprisingly, AtCOMTA160G showed repeatedly lower abundance in the purified fraction compared to wild-type AtCOMT, even though the concentration of the two enzymes was equal in the lysate (FIG. 24A). Therefore, a lysate expressing each enzyme with either 5-HT 5, 5-HNMT 6 or bufotenin 7 as substrates was tested. Since the inventors lacked an analytical standard for bufotenin 7, an extract from N. benthamiana leaves co-expressing AroG, PvTDC2, OsCYP71P1 and RmNMT, which served as the bufotenin 7 substrate in the enzyme assays, was used. Both AtCOMT and AtCOMTA160G exhibited weak activity with 5-HT 5 towards 5-MeO-TAM 8, with AtCOMTA160G showing only a modest 2-fold increase relative to the wild-type AtCOMT (FIGS. 24B-24C). In contrast, AtCOMTA160G exhibited markedly higher activity with 5-HNMT 6 and bufotenin 7 (FIGS. 24B-24C), yielding a 30-fold increase in 5-MeO-NMT 9 and 5-MeO-DMT 10, corroborating the results obtained in N. benthamiana. These results suggest that the A160G mutation effectively expanded the active site, or increased its local flexibility, enabling methylation of 5-HNMT 6 and bufotenin 7 to generate 5-MeO derivatives (FIG. 4).

The inventors then co-infiltrated PvNMT1 alongside AroG, PvTDC2, OsCYP71P1, RmNMT and either AtCOMT or AtCOMT-Mut1 hypothesizing that the combined expression might further increase 5-MeO-DMT 10 levels via N-methylation of 5-MeO-NMT 9. Although this strategy did not enhance 5-MeO-DMT 10 production, it resulted in a notable increase in DMT 4 levels compared to infiltration with RmNMT alone (3.3 μg·g⁻¹ of DMT 4 with PvNMT1; FIG. 5B). While this concentration was lower than that achieved with AroG, PvTDC2, and PvNMT1, the ability to co-produce multiple psychedelics in a single system underscores the platform's versatility.

### Example 6

Parallel Bioproduction of Psychedelics in N. benthamiana

The inventors next explored the feasibility of simultaneously producing all five natural psychedelics within a single N. benthamiana plant by co-expressing the most active genes from each pathway. Leaves were agroinfiltrated with a mixture of AroG, PvTDC2, PvNMT1, PsiH, PsiK, PsiM, OsCYP71P1, RmNMT and AtCOMT-Mut1. One week post-infiltration, all five targeted psychedelics were detected, though their concentrations were markedly reduced compared to those obtained from individual pathway expression (FIGS. 5A-5B versus 5D).

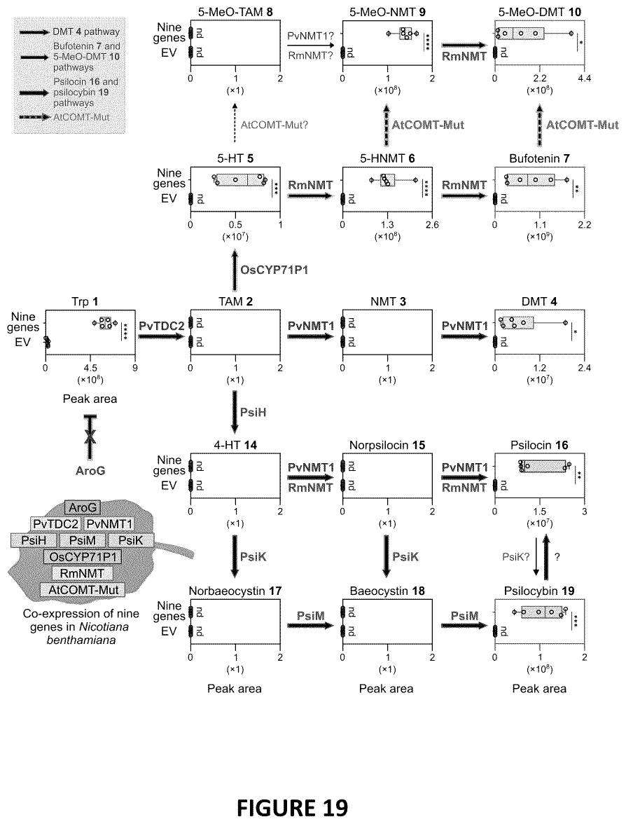

The most pronounced reduction was observed in DMT 4, which decreased from an average of 89.4 μg·g⁻¹ (in the AroG, PvTDC2 and PvNMT1 combination) to just 0.08 μg·g⁻¹ when all nine genes were co-expressed (FIG. 5D). Notably, several intermediates downstream of 5-HT 5 (molecules 5, 6 and 9, FIG. 19) accumulated in the samples, while intermediates from the DMT and psilocybin pathways (molecules 2, 3, 14, 15, and 17, FIG. 19) were undetectable. In addition, the concentrations of bufotenin 7 and 5-MeO-DMT 10 were both reduced by approximately 5-fold relative to their levels in plants expressing AroG, PvTDC2, OsCYP71P1, RmNMT, and AtCOMT-Mut1. These results suggest that OsCYP71P1 has high catalytic efficiency, diverting a significant portion of TAM 2 to 5-HT 5 through hydroxylation.

The relatively high accumulation of psilocybin 19 (9.8 ugg-1 on average) compared to DMT 4 also points to more efficient hydroxylation of TAM 2 by PsiH than N-methylation by PvNMT1 (FIG. 19). Additionally, accumulation of Trp 1 (FIG. 19) indicates that further increasing TAM 2 levels, potentially via co-expression of PvTDC1 or an alternative TDC, may enhance titers in future multiplexed production setups. Altogether, these findings demonstrate the complete in planta reconstruction of five natural psychedelic indolethylamines originating from plants, fungi, and animals within a single N. benthamiana host.

### Example 7

De Novo Bioproduction of Halogenated Indolethylamines in N. benthamiana

Several halogenated indolethylamine derivatives have demonstrated therapeutic potential for mental disorders. However, apart from a few brominated DMT analogs identified in marine organisms, naturally occurring halogenated indolethylamines remain exceedingly rare. Previous studies have shown that prokaryotic halogenases can selectively introduce carbon-specific halogenation into Trp 1 in planta, offering a strategy to expand the structural diversity of bioactive compounds.

To explore this approach for generating halogenated DMT derivatives, the inventors transiently expressed the three previously characterized tryptophan halogenases: RebH (7-halogenase from Lechevalieria aerocolonigenes, BAC15758), PyrH (5-halogenase from Streptomyces rugosporus, AY623051), or SttH (6-halogenase from Streptomyces toxitricini, HQ844046) in N. benthamiana. To enhance halogenation efficiency, the inventors also introduced RebF, an NADH-dependent flavin reductase from Lechevalieria aerocolonigenes (BAC15756). Additionally, the infiltration buffer containing magnesium chloride was supplemented with potassium bromide to facilitate the biosynthesis of both chlorinated and brominated derivatives.

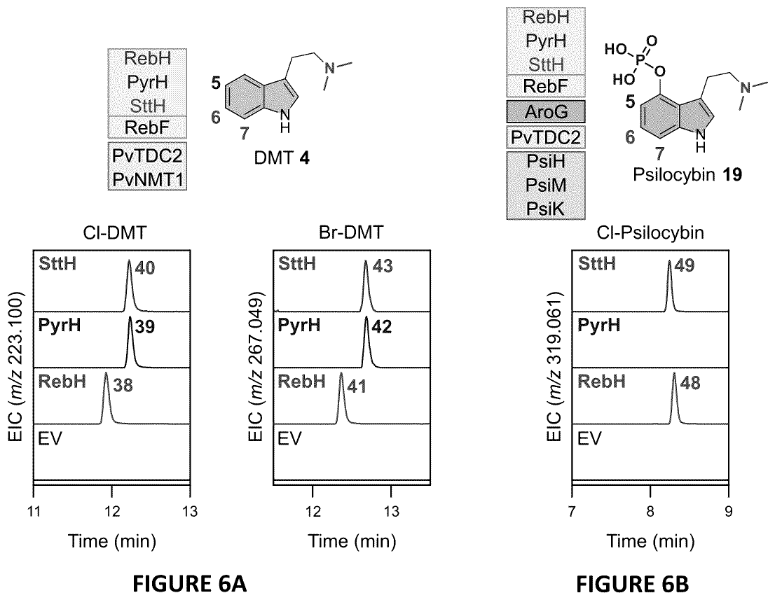

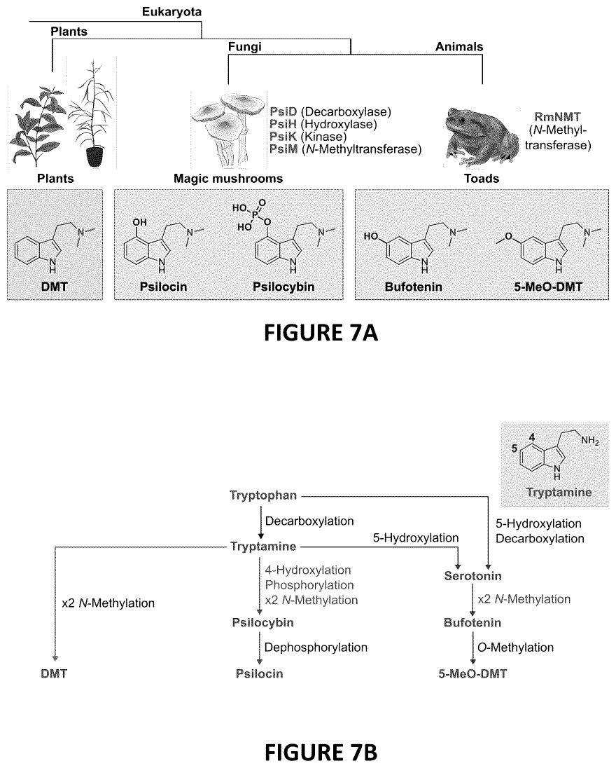

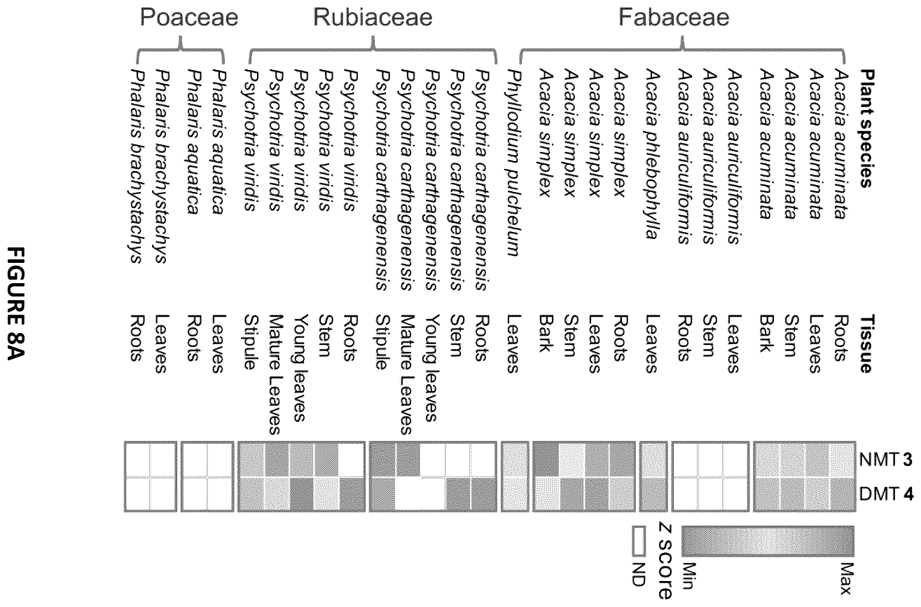

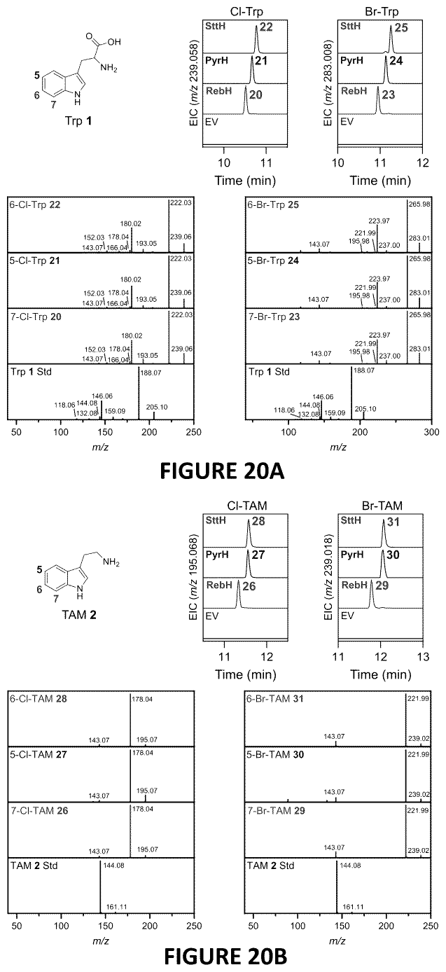

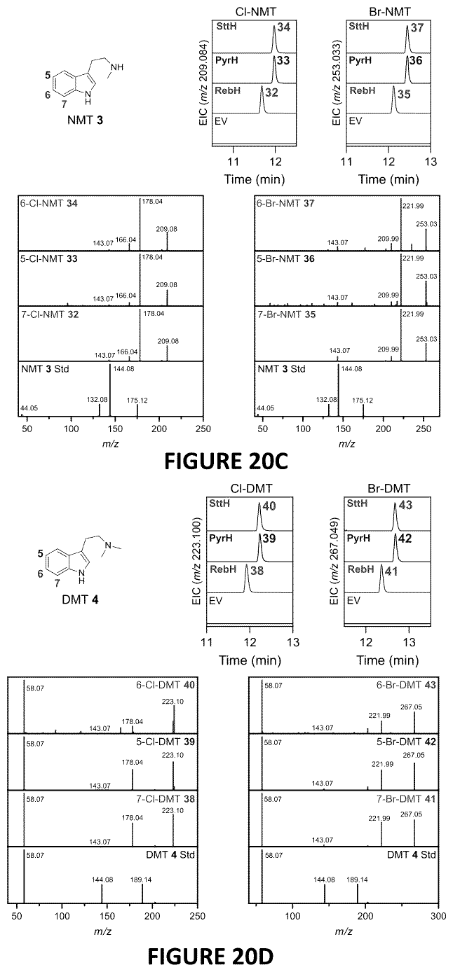

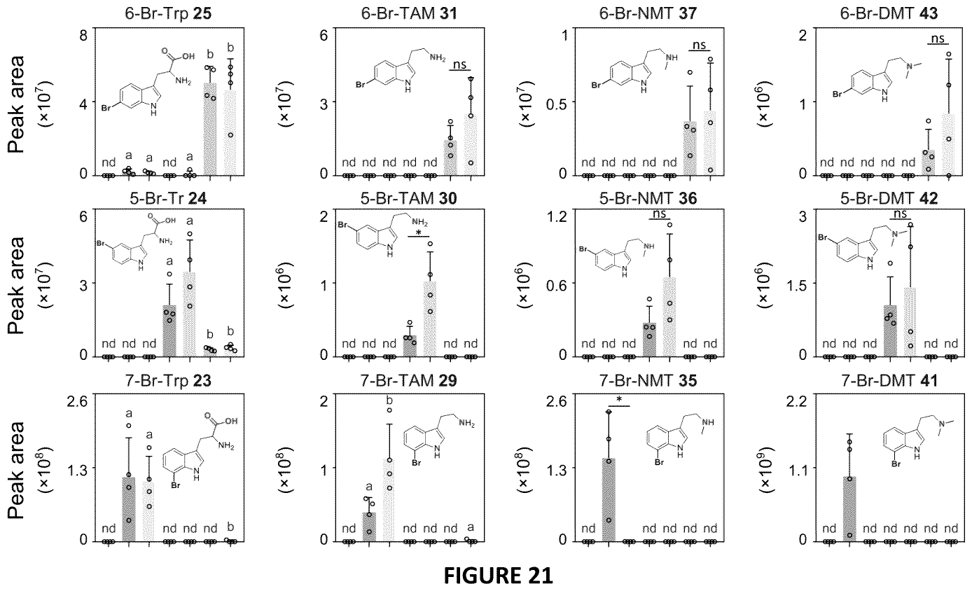

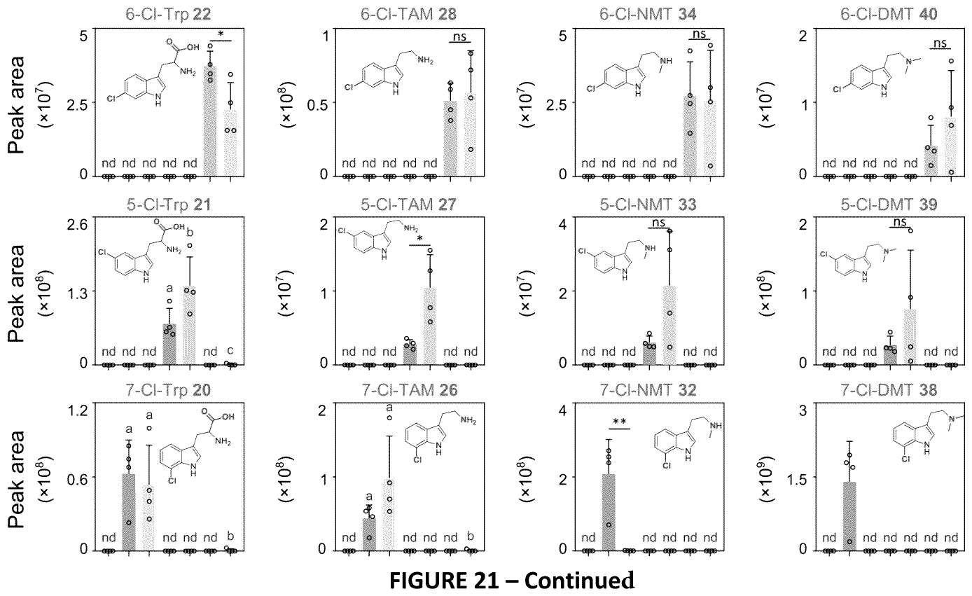

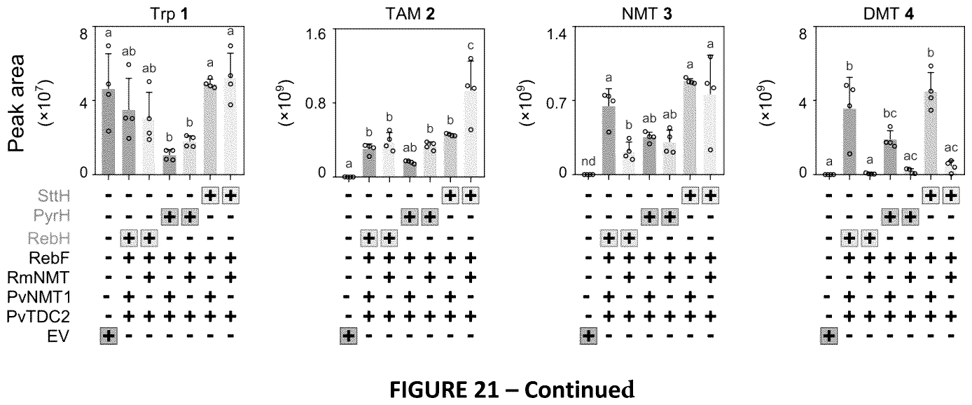

LC-MS analysis revealed distinct peaks corresponding to 7-, 5-, or 6-halogenated tryptophan, consistent with previous reports (molecules 20-25, FIG. 20A). Further co-expression with PvTDC2 and either PvNMT1 or RmNMT yielded halogenated tryptamine (26-31), NMT (32-37), and DMT (38-43) derivatives, with substitution patterns reflecting the halogenase used (FIGS. 6A, 20B-20D, 21). While RmNMT and PvNMT1 exhibited no significant differences for the production of 5-/6-halogenated-DMT derivatives (39, 40, 42 and 43), only PvNMT1 catalyzed the N-methylation of 7-Cl-DMT 38 or 7-Br-DMT 41, while also producing significantly higher concentrations of DMT 4 (FIG. 21).

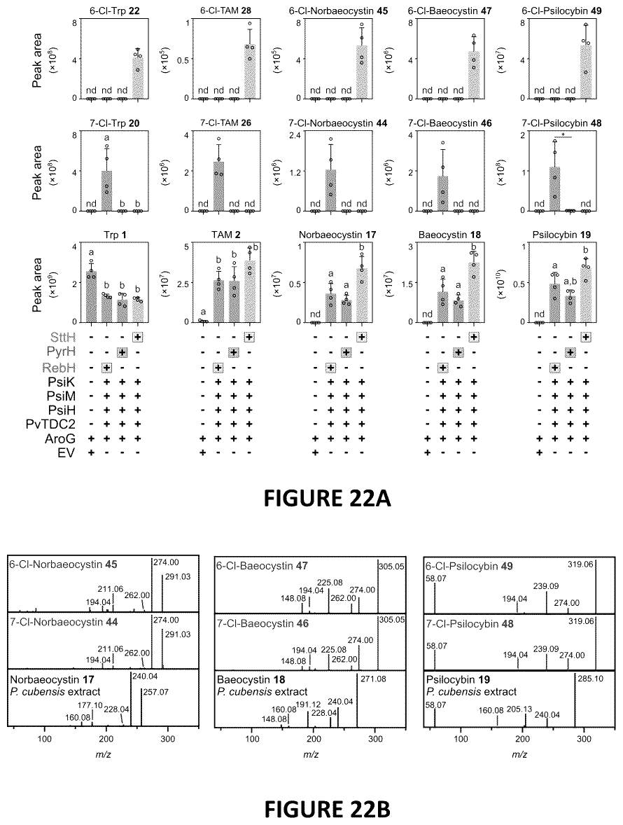

Agroinfiltration of AroG, PvTDC2, PsiH, PsiM, PsiK, RebF and either RebH, PyrH or SttH into N. benthamiana leaves produced 7-Cl- and 6-Cl-psilocybin in addition to the other phosphorylated intermediates (molecules 44-49, FIGS. 6B and 22). No 5-halogenation was observed for any of the phosphorylated molecules, suggesting that the phosphate group creates steric hindrance at this position.

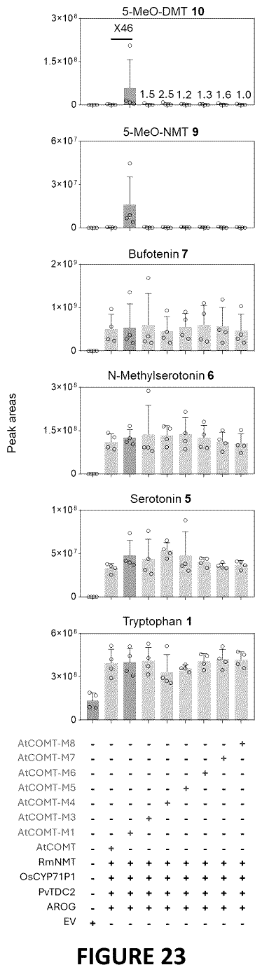

Further, the inventors tested the bioactivity of AtCOMT mutants via transient expression in Nicotiana benthamiana. Ion abundance peak areas of indolethylamine intermediates from the 5-methoxy-N,N-dimethyltryptamine (5-MeO-DMT 10) pathway were compared following transient expression of AroG, PvTDC2, OsCYP71P1, RmNMT, and either wild-type AtCOMT or one of its mutants (AtCOMT-Mut1, Mut3-Mut8) in N. benthamiana leaves. The specific amino acid substitutions for each mutant are listed under SEQ ID Nos: 14-20. Leaf samples were collected one week following agroinfiltration. The results show that AtCOMT-M1 (e.g., mutant #1; of SEQ ID Nos: 13-14) was highly active compared to the wild-type AtCOMT (SEQ ID Nos: 11-12), and the other mutants, for example, with respect to 5-MeO-NMT 9 and 5-MeO-DMT 10 synthesis (FIG. 23).

### DISCUSSION

The expanding clinical interest in psychedelics as therapeutics has underscored the need for scalable and versatile platforms for compound production and diversification. Previous efforts to biosynthesize indolethylamine psychedelics have focused primarily on microbial systems, typically targeting the production of a single compound or partial pathways. The elucidation of the psilocybin biosynthetic pathway in P. cubensis and its reconstruction in E. coli and Saccharomyces cerevisiae marked important milestones in microbial synthetic biology. However, gaps in the identification of key enzymatic steps have posed bottlenecks to the full reconstruction of other psychedelic biosynthetic pathways. The recent discovery of RmNMT from the cane toad, which exhibits broad substrate promiscuity for N-methylation of diverse indolethylamines, has expanded the toolkit for heterologous biosynthesis; however, it has yet to be incorporated into the full reconstruction of a complete biosynthetic pathway in vivo. Although psychedelic idolethylamine biosynthetic pathways share precursor molecules and exhibit parallel enzymatic reactions, no study to date has attempted the reconstruction of multiple natural and novel analogs within a unified expression system. Here the inventors exploited N. benthamiana as an efficient platform for transient expression and successfully reconstructed complete biosynthetic pathways of tertiary indolethylamines with therapeutic potential (FIG. 4).

The inventors started by identifying active TDC and NMT enzymes responsible for the accumulation of DMT 4 in P. viridis and A. acuminata. The simplicity of this biosynthetic pathway, comprising only two reaction types, may underline the broad occurrence of DMT 4 across phylogenetically diverse plant families. Yet its accumulation is not conserved even among closely related species (FIG. 8). For instance, P. carthagenensis, a taxonomical close relative of P. viridis, showed homologues of PvTDC1, PvTDC2, and PvNMT1, but their low expression across tissues correlated with the absence of DMT 4 (FIG. 1B). Likewise, while A. acuminata, Acacia phlebophylla, and Acacia simplex accumulated substantial concentrations of both NMT 3 and DMT 4, Acacia auriculiformis lacked detectable levels of either compound (FIG. 8). These findings point to lineage-specific regulation or loss of enzymatic activity and highlight the need for comparative genomic and transcriptomic analyses across related species to elucidate the evolutionary origins and diversification of this pathway.

In vitro biochemical assays revealed that PvNMT1 and AaNMT1 exhibit remarkable substrate promiscuity. In addition to their natural substrate TAM 2, both enzymes accepted a wide range of hydroxylated and methoxylated tryptamines, demonstrating their potential as versatile biocatalysts for heterologous psychedelic biosynthesis. In N. benthamiana, PvNMT1 yielded the highest concentrations of DMT 4, while RmNMT was more effective in producing bufotenin 7, consistent with their roles in the native producers P. viridis and R. marina, respectively. Both enzymes also catalyzed the biosynthesis of psilocin 16, offering an alternative route that bypasses phosphorylation, though production levels remained modest (average of 0.64 μg·g⁻¹ fresh weight). By contrast, PsiM, the NMT from P. cubensis, displayed strict substrate specificity, requiring phosphorylated intermediates for activity. Its co-expression was essential for psilocybin 19 biosynthesis, reinforcing the enzyme's role in the canonical fungal pathway (FIGS. 14A-14C). Given its greater chemical stability and substantially higher titers achieved in the engineered N. benthamiana host, psilocybin 19 presents a more effective route for psilocin 16 production, acting as a prodrug that is enzymatically dephosphorylated to psilocin 16 in vivo.

Although the biosynthetic pathways of bufotenin 7 and 5-MeO-DMT 10 remain only partially elucidated, the incorporation of functionally characterized enzymes from rice and A. thaliana, two species that do not naturally produce psychedelics, enabled their de novo reconstruction in N. benthamiana. Notably, bufotenin 7 accumulated at high levels (average of 42.8 μg·g⁻¹ fresh weight), whereas 5-MeO-DMT 10 accumulated at significantly lower levels (average of 0.21 μg·g⁻¹ fresh weight), suggesting limited catalytic efficiency of AtCOMT in this pathway. Structure-guided mutagenesis of a single AtCOMT residue resulted in a forty-fold enhancement of 5-MeO-DMT 10 production. Future identification of native OMTs from the Sonoran Desert toad or plants known to accumulate this metabolite may further enhance its biosynthetic efficiency.

The current work further expands the biosynthetic capacity of the plant chassis by integrating bacterial flavin-dependent halogenases, enabling regioselective chlorination and bromination of DMT and psilocybin derivatives. Given that RebH and PyrH do not halogenate TAM 2, the successful production of halogenated tertiary indolethylamines suggests that downstream enzymes in the pathways exhibit broad substrate promiscuity, accepting halogenated intermediates alongside their native counterparts. This approach opens new avenues for generating halogenated analogs with distinct pharmacological properties and could be extended to include other halides, such as fluorine or iodine, novel indolethylamine scaffolds, or combinatorial biosynthetic designs. Several halogenated indolethylamines have already shown therapeutic promise. For instance, 5-chloro- and 5-fluoro-N,N-dimethyltryptamine elicit robust head-twitch responses in mice, a behavior that correlates to hallucinogenic activity in humans. In contrast, 5-bromo-N,N-dimethyltryptamine exhibits sedative effects, while its 5,6-dibromo analog demonstrates antidepressant-like properties. Fluorination at the 6- or 7-position of psilocin 16 has also been reported to enhance 5-HT_{2A} receptor affinity. Together, these findings underscore the utility of the current platform for probing structure-activity relationships and accelerating the development of next-generation psychedelic therapeutics.

Several halogenated indolethylamines have already shown therapeutic potential. For instance, 5-chloro- and 5-fluoro-N,N-dimethyltryptamine elicit strong head-twitch responses in mice, a behavior that correlates to hallucinogenic activity in humans. In contrast, 5-bromo-N,N-dimethyltryptamine displays sedative effects, while its 5,6-dibromo analog exhibits antidepressant-like properties. Fluorination at the 6 or 7 position of psilocin 16 has also been reported to enhance affinity for the 5-HT_{2A} receptor. These findings underscore the value of the current system as a modular platform for probing structure-activity relationships and accelerating the discovery and optimization of next-generation psychedelic therapeutics.

The successful reconstruction of five distinct psychedelic pathways within a single N. benthamiana host underscores the potential of plant-based systems for parallel biosynthesis of diverse indolethylamines. While titers of individual compounds were lower than those observed in single-pathway expression, likely due to precursor competition, metabolic crosstalk, and imbalanced enzyme expression, the ability to simultaneously produce multiple natural psychedelics, along with halogenated analogs, marks a key milestone for scalable biosynthesis and combinatorial pathway engineering. Future improvements may include rational protein design, dynamic expression control, flux optimization, and spatial compartmentalization to enhance yields. Further work is also needed to address metabolite stability, bioavailability, and downstream extraction. Altogether, this study establishes a blueprint for multiplexed in planta production of structurally and functionally diverse psychedelics, paving the way for therapeutic innovation and sustainable biosynthetic platforms.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

What is claimed is:

1. An artificial DNA molecule comprising a nucleic acid sequence having at least 80% identity or homology to SEQ ID NO: 1 or SEQ ID NO: 2.

2. The artificial DNA molecule of claim 1, wherein said nucleic acid sequence encodes a protein being a decarboxylase.

3. The artificial DNA molecule of claim 2, wherein said decarboxylase is tryptophan decarboxylase.

4. An artificial DNA molecule comprising a nucleic acid sequence having at least 80% identity or homology to SEQ ID NO: 3.

5. An artificial DNA molecule comprising a nucleic acid sequence having at least 80% identity or homology to SEQ ID NO: 7.

6. The artificial DNA molecule of claim 5, wherein said nucleic acid sequence encodes a protein being a methyltransferase, and optionally wherein said methyltransferase is N-methyltransferase (NMT).

7. A vector comprising the artificial DNA molecule of claim 1.

8. The vector of claim 7, being a plasmid or an agrobacterium.

9. The vector of claim 7, further comprising at least one additional nucleic acid sequence encoding at least one enzyme selected from the group consisting of: CYP71P1, O-methyltransferase (OMT), PsiH, PsiK, PsiM, and any combination thereof.

10. A polypeptide encoded by the vector of claim 7.

11. The polypeptide of claim 10, being a recombinant polypeptide.

12. The polypeptide of claim 10, comprising an amino acid sequence with at least 85% homology to any one of SEQ ID Nos: 4-6, and 8.

13. The polypeptide of claim 10, characterized by having an activity of releasing a carboxyl group from a substrate.

14. A transgenic or transfected cell comprising the artificial DNA molecule of claim 1.

15. An extract, lysate, or homogenate derived from the transgenic or transfected cell of claim 14, or any fraction thereof.

16. A composition comprising the extract of claim 15 and an acceptable carrier.

17. A method for synthesizing tryptamine or a metabolite thereof, the method comprising:
a. providing a cell comprising said artificial DNA molecule of claim 1; and
b. culturing said cell from step (a) such that a first polypeptide encoded by said artificial DNA molecule is expressed,
thereby synthesizing tryptamine.

18. The method of claim 17, wherein: (i) said culturing comprises supplementing said cell with an effective amount of tryptophan; (ii) said cell further comprises at least one second artificial DNA molecule comprising a nucleic acid sequence having at least 80% homology to any one of SEQ ID Nos: 3, 7, and 10, and said step (b) comprises culturing said cell from step (a) such that a second polypeptide encoded by said at least one second artificial DNA molecule is expressed, thereby synthesizing N-methyltryptamine (NMT), N,N-dimethyltryptamine (DMT), or both; or both (i) and (ii).

19. The method of claim 17, wherein said cell further comprises at least one third artificial DNA molecule comprising a nucleic acid sequence encoding at least one enzyme selected from the group consisting of: CYP71P1, 4-Hydroxylase, 5-Hydroxylase, PsiH, PsiK, OMT, or any combination thereof, and said step (b) comprises culturing said cell from step (a) such that at least one third polypeptide encoded by said at least one third artificial DNA molecule is expressed, thereby synthesizing said tryptamine metabolite being selected from the group consisting of: Serotonin, 5-methoxytryptamine, 4-OH-Trytamine, Norbaeocystin, 5-methoxy-N-methyltryptamine (5-MeO-NMT), N-methyl-Serotonin (N-Me-Serotonin), Norpsilocin, Baeocystin, 5-methoxy-DMT (5-MeO-DMT), Bufotenine, Psilocin, Psilocybin, and any combination thereof, optionally wherein said OMT is Arabidopsis thaliana OMT or a mutant thereof, and optionally wherein said mutant comprises a single mutation compared to said AtOMT, and is characterized by increased activity compared thereto, and optionally wherein said activity is synthesis of 5-MeO-NMT, 5-MeO-DMT, or both, and optionally said synthesis is of 5-MeO-DMT, and said mutant activity is increased by at least 5-fold compared to said AtOMT.


SEQUENCE LISTING (26 sequences)

SEQ ID NO: 1 - DNA, 1518 bp, Psychotria viridis
  source 1..1518: mol_type = other DNA
atggatagtcttgatgaaaacaatgtctccttttccccaccacaattagacaccgaattcaagccgctcgacattcaagaattccgtacacaagcccatcgaatggtagacttcatagccgattactaccaaaacatcgagaactaccctgtcctcagccaagttgaaccggggcacctccgaaaccgtttacctgaatctgccccttaccggcccgaaccctttgagaacattctcagagacataaaaacaagcatcatccctgtaatgactcattggctaagcccaaatttcttcgcgttctttcccgccaccgtcagctctgccgcttttgttggggaaatgctctgcacgtgcttcaacgccgtgggtttcaactggctggcttctcccgcaaccactgagctcgagatggtggttatggattggctggcacagctattgatgcttcccaagtcattcatgttttcgggctccggtggcggagttatccagggaaccaccagcgagtccatcctcgttgctctcatcgccgcccgggatcgggcactggcggtcattggagcccacggaatatcaaagcttgtagtttacggctccgaccagactcattcttcgttcgcgaagacttgtaaggtggcaggtattttcccatgcaacatacgaagtattcctacgaccgccgaaaacaactttgctttttcccccgcggtcctacgtagggtgattgaggctgatgtggcttcagggatggtcccactttttatatgcgctaccattgggaccacttccactactgccattgacccccttagtcaactagctgacgtggccaacgatcacggagtgtggcttcacgtggacgcggcctacggcggcagcgcatgcatttgtcccgagtttcggcactatctggacgggattgagcgagtcgactcgctgagtctgagcccccataagtggctactcagttacttggactgctgttgcttgtgggttaaacaacccaactcgttagtcaaggcactgagtacaaatccggagtacttgagaaacaaaatgagcgagttgggttccgtggtggattacaaggattggcaagtgggcacaggtcgacggtttaaagcgctccgattatggctgatcatgcgtagttatggcgtcgcaaacctccaaagccacattcgatccgacgtccgaatggcgaagatgtttgaagagtttataaattccgacccgaggttcgaagtagtggtgccgagaaccttctcgctagtgtgttttaggtttaacccgtacaaagagtccgacaaggcatacatcaaccacttgaacaagaagctgctagagtcggttaactcaacgggtcgactgtacatgactcacacagtggcgggggacgcgtttgtgctgagatttgcagtgggggcgactctcaccgaagaccgccacatatgtgccgcttgggagttaataaaagagtcagcggaggctttgctgaagaacaatgttgtctga

SEQ ID NO: 2 - DNA, 1530 bp, Psychotria viridis
  source 1..1530: mol_type = other DNA
atgggcaccattgatacaaatgatggtgcatatcctgattcaatattcaccccattcaatcctcttgatcctgaagaattcagaaaaaaggcccatattatggttgatttcatagctgactactacaaaaatatcgagaactaccctgttttgagccaagttgaacccggatacctcagaacccacttgcccaaaaccgcaccgtatttgcccgaatcatttgaaaccattctcagtgatattcagaaagatataatcccaggaatgacaaactggttaagtcctaattttttcgctttttttcccgccacggttagctcggctgccttccttggagaaatgttatgcacaggtttcaactctgtagggttcaactggctcgcctccccggctgccaccgagctggagatggttgtcatggattggatggctaagatgttaaagttgcccaaaacattcatgttttcaggcacaggtggtggcgttcttcaaggaaccaccagtgagtccattctctgcgtcctcatcgccgcccgcgaccgcgccttcgagagaataggcatccagaacattggcaagctcgttgcgtacggatccgatcaaactcactcattcttccaaaagacgtgcaagttggcgggtattttcccatgcaacgtcaaaataatccccacgacaagggaagcaaactttaccttgtccccagcagccctacgcagacaaatcgaagcggacctgacagatggcctggtcccactattcctatgtgcaactgttgggaccacttcgaccacggcggtggacccggtgggcgagctcgccaaagtggcgaacgatttcaacgtctgggtccacgtcgatgccgcgtatgccggcagcgcatgcatatgccctgaatttcagcaatacttggatggggtggagagggctgattctttaagtttgagccctcataaatggctactttcttatttggattgttgttgcttgtgggtcaaggaacccgactcgatggtcaaggcattaagcaccaaccccgagtacttgagaaacaaaagaagcgagtttggctcggtggttgactttaaagattggcaaattgggaccggaaggcgatttagggcacttcgactatggctaattatgcgtagttatggcgttgctaacctccaagatcacattaggtctgacattcaaatggccaagatgttcgaagggttcgtgagatctgacccgaggttcgaactggttgtacctcgtgcattttctcttgtatgtttcaggctcaaatccatgagagaatccagcgaaccgtacacggaactcctgaacaagaagctactggagctggtcaactcgacgggccgggcctatatgacgcataccaaagttggtgggacttacatgctaagatttactgtgggagccacgttaacagaggagcgccacgtatgcgctgcttgggaattaatcacagaatgcactgacgttttactcaaggaatttcatttaccaggtctcccaaaatga

SEQ ID NO: 3 - DNA, 1056 bp, Psychotria viridis
  source 1..1056: mol_type = other DNA
atggattctgctttgagttctttatcgactcatgagcttcttagagctcaaacacacgtttggaaacaaacattcagctacaggaattctgcctgtctaaaatgtgcagttgagctaggcatcccagacgccatccaacaatatggcaagcccatgactctttctgagctgacttctgccttgcccatcaacccctccaaagctccctacattcaccgtctaatgcgcattttagtaaatgctggcattttttctgaacaaaaggagggctttgccctcacttctgccggccgacttcttgtcaaagatgatcctctcaataatacggcatttgttctgttggttcttgatcctgccataatgaaggcatggaattccttgaaagaatggttccagaatgatgatctcgccgcatttgacactgcacaccgcgtgggtttctgggattataatcatcgtgtaccccaatttggtaagctgtttgatgacgcaatggctagtgattcactgttgaccacaaaggtgttggttactgagtgcaagtatgtgtttgaagggttgacatctctagtggatgttggaggtggcaatgggacagttactagggccattgcaaaaactttccccaacttaaagtgtactgtgtatgatctcccacatgttgttgcccgccaagaaggagaggggaacttggattttgttgcaggagatatgtttgataaagttcccagtgccaacgcaatcctactcaagtggattctggatggctggggtatggaagattgtttgaagattctcaaaaactgcaggagggcaattccagggaaagagaaaggtggtaaagtgattgctatagaaatggtaatggacggcccagagctagatgctgagacaactgaagcacaaattaactcggacatggaggcggtggtcctcctccgcgctggagaaaaaactgaggaagaatttgcaatgctgtgtcgggatgctggcttctctagctataaaatatttccggtgttgggtacaagaagtatcattgagatgtatccatga

SEQ ID NO: 4 - protein, 505 aa, Psychotria viridis
MDSLDENNVSFSPPQLDTEFKPLDIQEFRTQAHRMVDFIADYYQNIENYPVLSQVEPGHLRNRLPESAPYRPEPFENILRDIKTSIIPVMTHWLSPNFFAFFPATVSSAAFVGEMLCTCFNAVGFNWLASPATTELEMVVMDWLAQLLMLPKSFMFSGSGGGVIQGTTSESILVALIAARDRALAVIGAHGISKLVVYGSDQTHSSFAKTCKVAGIFPCNIRSIPTTAENNFAFSPAVLRRVIEADVASGMVPLFICATIGTTSTTAIDPLSQLADVANDHGVWLHVDAAYGGSACICPEFRHYLDGIERVDSLSLSPHKWLLSYLDCCCLWVKQPNSLVKALSTNPEYLRNKMSELGSVVDYKDWQVGTGRRFKALRLWLIMRSYGVANLQSHIRSDVRMAKMFEEFINSDPRFEVVVPRTFSLVCFRFNPYKESDKAYINHLNKKLLESVNSTGRLYMTHTVAGDAFVLRFAVGATLTEDRHICAAWELIKESAEALLKNNVV

SEQ ID NO: 5 - protein, 509 aa, Psychotria viridis
MGTIDTNDGAYPDSIFTPFNPLDPEEFRKKAHIMVDFIADYYKNIENYPVLSQVEPGYLRTHLPKTAPYLPESFETILSDIQKDIIPGMTNWLSPNFFAFFPATVSSAAFLGEMLCTGFNSVGFNWLASPAATELEMVVMDWMAKMLKLPKTFMFSGTGGGVLQGTTSESILCVLIAARDRAFERIGIQNIGKLVAYGSDQTHSFFQKTCKLAGIFPCNVKIIPTTREANFTLSPAALRRQIEADLTDGLVPLFLCATVGTTSTTAVDPVGELAKVANDFNVWVHVDAAYAGSACICPEFQQYLDGVERADSLSLSPHKWLLSYLDCCCLWVKEPDSMVKALSTNPEYLRNKRSEFGSVVDFKDWQIGTGRRFRALRLWLIMRSYGVANLQDHIRSDIQMAKMFEGFVRSDPRFELVVPRAFSLVCFRLKSMRESSEPYTELLNKKLLELVNSTGRAYMTHTKVGGTYMLRFTVGATLTEERHVCAAWELITECTDVLLKEFHLPGLPK

SEQ ID NO: 6 - protein, 351 aa, Psychotria viridis
MDSALSSLSTHELLRAQTHVWKQTFSYRNSACLKCAVELGIPDAIQQYGKPMTLSELTSALPINPSKAPYIHRLMRILVNAGIFSEQKEGFALTSAGRLLVKDDPLNNTAFVLLVLDPAIMKAWNSLKEWFQNDDLAAFDTAHRVGFWDYNHRVPQFGKLFDDAMASDSLLTTKVLVTECKYVFEGLTSLVDVGGGNGTVTRAIAKTFPNLKCTVYDLPHVVARQEGEGNLDFVAGDMFDKVPSANAILLKWILDGWGMEDCLKILKNCRRAIPGKEKGGKVIAIEMVMDGPELDAETTEAQINSDMEAVVLLRAGEKTEEEFAMLCRDAGFSSYKIFPVLGTRSIIEMYP

SEQ ID NO: 7 - DNA, 1092 bp, Acacia acuminata
  source 1..1092: mol_type = other DNA
atggaatcccaaaacgaagggcgtgaagcagcaacaagaagagtgcttgaagctcaaacccatgtatggaaccacatgttcggctacattaactcaatgactttgaggtcagcaatagagttaagcataccagacatcattcacaagcatggcaagcccatgcctcttcatcttcttgcctcttcccttcaaatccaaccttccaaaacccctcacctccgtcgcttgatgcgcttcctcactcataccggctacttctctctccagaagcttccagaaacttctgacgaccctgaaggctacgtcctcactgatgcatctgccctttttctcaaggaccatccctttgctctcgcctcctgtactcttttcgcggttgacccactctcggtgaaaccgtgggaacatttgcccgcttggtttaagaacaatgatccatcgccgtttgaaacggcgtatggaaaggctttctatgactatgccgggaatgtgcctaggggtttcaattacttcaaagacacaatggctggcgattcaaaattggtggtaaaagtgttgcttgagcggtgtagggatgtgtttaagggatgtggctcgcttgttgatgttgggggtagtactggcgccatggccaaggtcgtagctgaagcttttccacaaatggagtgtactgtgtttgatttgccttatgttgttggtggcttaaaggactgtgggaacctcaagttcgtccaaggagacatgtttcaaactattcctccggccgatattctcatgatgaagtggattctaagtgactgggacgatgagagttgtctgaaattactcaagaactgcaaggaggcagtaacaagcaaaggcaagcgagggaagttggttataataaacgaagtgattgaggatgacaaggcagattatgatgatgaatcggttgaaacacagttgaaatttgatctggcaataatgacactgttaacaggaaaagagagaaatgagaaagagtgggcccacttgttttcctctgcaggtttcactacctataaaataactccagttctaggtgtaaggtctctcattgaggttttcccataa

SEQ ID NO: 8 - protein, 363 aa, Acacia acuminata
MESQNEGREAATRRVLEAQTHVWNHMFGYINSMTLRSAIELSIPDIIHKHGKPMPLHLLASSLQIQPSKTPHLRRLMRFLTHTGYFSLQKLPETSDDPEGYVLTDASALFLKDHPFALASCTLFAVDPLSVKPWEHLPAWFKNNDPSPFETAYGKAFYDYAGNVPRGFNYFKDTMAGDSKLVVKVLLERCRDVFKGCGSLVDVGGSTGAMAKVVAEAFPQMECTVFDLPYVVGGLKDCGNLKFVQGDMFQTIPPADILMMKWILSDWDDESCLKLLKNCKEAVTSKGKRGKLVIINEVIEDDKADYDDESVETQLKFDLAIMTLLTGKERNEKEWAHLFSSAGFTTYKITPVLGVRSLIEVFP

SEQ ID NO: 9 - protein, 263 aa, Rhinella marina
MENSYSAQMYIDEFDPVHYYQTYYSSGKGGIAREWTDFALQNLHETFGPGGVKGDILIDFGAGPTIYQLLSACEVFNSIITSDFLEQNREQLEKWLRKDPDALDWSHFTKYVCELEGNRDNWEKKEETLRRKVTKVLKCDALAEKPFDDVPMPEADCLISCLCLENPCQDQEAYINILKKLKELLKPGGHIIIQSILNCSYYHIGNSCFSHLSLSKDDVEKSFKEAGYEIVKLKVLPRSVMSEMEISDSNGYYFIHARKPQKE

SEQ ID NO: 10 - DNA, 792 bp, Rhinella marina
  source 1..792: mol_type = other DNA
atggagaactcatactctgcacagatgtacattgatgaatttgatcctgtacactactaccagacatactattcttcggggaaaggaggtatcgctagagaatggacagactttgccttacagaatctgcatgaaacatttggtccaggaggtgtgaaaggagacatactaattgactttggagctggacccacaatttatcaactgctctctgcttgtgaagtgttcaacagcattataacctctgattttcttgagcaaaaccgagagcaattggaaaaatggctgagaaaggacccagatgccttagactggtctcattttaccaaatatgtgtgtgaactggaaggtaacagagataactgggaaaaaaaggaagaaacgctgaggagaaaagtaaccaaagtgttgaagtgtgacgctcttgcggagaagccatttgacgacgtgcctatgccagaagccgactgtctgattagctgcctctgcttagaaaatccttgtcaagaccaagaagcttatatcaatatattgaaaaagcttaaggagctacttaaacccggtggtcacattataattcagagtattcttaattgttcatactaccatattggtaacagctgtttttcccatctgtccttatctaaggatgacgtggaaaaatcatttaaagaggccgggtatgaaattgttaaattaaaggtgctgccacgatctgtaatgtcagaaatggagatcagtgattctaatggctattactttatccatgctcgtaaaccacagaaagagtaa

SEQ ID NO: 11 - DNA, 1157 bp, Arabidopsis thaliana
  source 1..1157: mol_type = other DNA
atgggttcaacggcagagacacaattaactccggtgcaagtcaccgacgacgaagctgccctcttcgccatgcaactagccagtgcttccgttcttccgatggctttaaaatccgccttagagcttgaccttcttgagattatggccaagaatggttctcccatgtctcctaccgagatcgcttctaaacttccgaccaaaaatcctgaagctccggtcatgctcgaccgtatcctccgtcttcttacgtcttactccgtcttaacctgctccaaccgtaaactttccggtgatggcgttgaacggatttacgggcttggtccggtttgcaagtatttgaccaagaacgaagatggtgtttccattgctgctctttgtcttatgaaccaagacaaggttctcatggaaagctggtaccatttgaaggatgcaattcttgatggtgggattccattcaacaaggcttatggaatgagcgcgttcgagtaccacgggactgaccctagattcaacaaggtctttaacaatggaatgtctaaccattccacaatcaccatgaagaagattcttgagacctataagggttttgaaggattgacttctttggttgatgttggtggtggcattggtgctacactcaaaatgattgtctccaagtaccctaatcttaaaggcatcaactttgatctcccacatgtcatcgaagatgctccttctcatcctggtattgagcatgttggaggagatatgtttgtaagtgtccctaaaggtgatgccatattcatgaagtggatatgtcatgactggagtgacgaacattgcgtgaaattcttgaagaactgctacgagtcacttccagaggatggaaaagtgatattagcagagtgtatacttccagagacaccagactcaagcctctcaaccaaacaagtagtccatgtcgattgcattatgttggctcacaatcccggaggcaaagaacgaaccgagaaagagtttgaggcattagccaaagcatcaggcttcaagggcatcaaagttgtctgcgacgcttttggtgttaaccttattgagttactcaagaagctctaaaaacaaacaatgttcctatgaagatgatttatatgtaaacattatctcatatctccttccacggt

SEQ ID NO: 12 - protein, 363 aa, Arabidopsis thaliana
MGSTAETQLTPVQVTDDEAALFAMQLASASVLPMALKSALELDLLEIMAKNGSPMSPTEIASKLPTKNPEAPVMLDRILRLLTSYSVLTCSNRKLSGDGVERIYGLGPVCKYLTKNEDGVSIAALCLMNQDKVLMESWYHLKDAILDGGIPFNKAYGMSAFEYHGTDPRFNKVFNNGMSNHSTITMKKILETYKGFEGLTSLVDVGGGIGATLKMIVSKYPNLKGINFDLPHVIEDAPSHPGIEHVGGDMFVSVPKGDAIFMKWICHDWSDEHCVKFLKNCYESLPEDGKVILAECILPETPDSSLSTKQVVHVDCIMLAHNPGGKERTEKEFEALAKASGFKGIKVVCDAFGVNLIELLKKL

SEQ ID NO: 13 - DNA, 1157 bp, Synthetic construct
  source 1..1157: mol_type = other DNA
atgggttcaacggcagagacacaattaactccggtgcaagtcaccgacgacgaagctgccctcttcgccatgcaactagccagtgcttccgttcttccgatggctttaaaatccgccttagagcttgaccttcttgagattatggccaagaatggttctcccatgtctcctaccgagatcgcttctaaacttccgaccaaaaatcctgaagctccggtcatgctcgaccgtatcctccgtcttcttacgtcttactccgtcttaacctgctccaaccgtaaactttccggtgatggcgttgaacggatttacgggcttggtccggtttgcaagtatttgaccaagaacgaagatggtgtttccattgctgctctttgtcttatgaaccaagacaaggttctcatggaaagctggtaccatttgaaggatgcaattcttgatggtgggattccattcaacaaggcttatggaatgagcgggttcgagtaccacgggactgaccctagattcaacaaggtctttaacaatggaatgtctaaccattccacaatcaccatgaagaagattcttgagacctataagggttttgaaggattgacttctttggttgatgttggtggtggcattggtgctacactcaaaatgattgtctccaagtaccctaatcttaaaggcatcaactttgatctcccacatgtcatcgaagatgctccttctcatcctggtattgagcatgttggaggagatatgtttgtaagtgtccctaaaggtgatgccatattcatgaagtggatatgtcatgactggagtgacgaacattgcgtgaaattcttgaagaactgctacgagtcacttccagaggatggaaaagtgatattagcagagtgtatacttccagagacaccagactcaagcctctcaaccaaacaagtagtccatgtcgattgcattatgttggctcacaatcccggaggcaaagaacgaaccgagaaagagtttgaggcattagccaaagcatcaggcttcaagggcatcaaagttgtctgcgacgcttttggtgttaaccttattgagttactcaagaagctctaaaaacaaacaatgttcctatgaagatgatttatatgtaaacattatctcatatctccttccacggt

SEQ ID NO: 14 - protein, 363 aa, Synthetic construct
MGSTAETQLTPVQVTDDEAALFAMQLASASVLPMALKSALELDLLEIMAKNGSPMSPTEIASKLPTKNPEAPVMLDRILRLLTSYSVLTCSNRKLSGDGVERIYGLGPVCKYLTKNEDGVSIAALCLMNQDKVLMESWYHLKDAILDGGIPFNKAYGMSGFEYHGTDPRFNKVFNNGMSNHSTITMKKILETYKGFEGLTSLVDVGGGIGATLKMIVSKYPNLKGINFDLPHVIEDAPSHPGIEHVGGDMFVSVPKGDAIFMKWICHDWSDEHCVKFLKNCYESLPEDGKVILAECILPETPDSSLSTKQVVHVDCIMLAHNPGGKERTEKEFEALAKASGFKGIKVVCDAFGVNLIELLKKL

SEQ ID NO: 15 - protein, 363 aa, Synthetic construct
MGSTAETQLTPVQVTDDEAALFAMQLASASVLPMALKSALELDLLEIMAKNGSPMSPTEIASKLPTKNPEAPVMLDRILRLLTSYSVLTCSNRKLSGDGVERIYGLGPVCKYLTKNEDGVSIAALCLMNQDKVLMESWYHLKDAILDGGIPFNKAYGMSGFEYIGTDPRFNKVFNNGMSNHSTITMKKILETYKGFEGLTSLVDVGGGIGATLKMIVSKYPNLKGINFDLPHVIEDAPSHPGIEHVGGDMFVSVPKGDAIFMKWICHDWSDEHCVKFLKNCYESLPEDGKVILAECILPETPDSSLSTKQVVHVDCIMLAHNPGGKERTEKEFEALAKASGFKGIKVVCDAFGVNLIELLKKL

SEQ ID NO: 16 - protein, 363 aa, Synthetic construct
MGSTAETQLTPVQVTDDEAALFAMQLASASVLPMALKSALELDLLEIMAKNGSPMSPTEIASKLPTKNPEAPVMLDRILRLLTSYSVLTCSNRKLSGDGVERIYGLGPVCKYLTKNEDGVSIAALCLMNQDKVLMESWYHLKDAILDGGIPFNKAYGMSGFEYIGTDPRFNKVFNNGMSNHSTITMKKILETYKGFEGLTSLVDVGGGIGATLKMIVSKYPNLKGINFDLPHVIEDAPSHPGIEHVGGDMFVSVPKGDAIFMKWICHDWSDEHCVKFLKNCYESLPEDGKVILAECILPETPDSSLSTKQVVHVDCIMLAHSPGGKERTEKEFEALAKASGFKGIKVVCDAFGVNLIELLKKL

SEQ ID NO: 17 - protein, 363 aa, Synthetic construct
MGSTAETQLTPVQVTDDEAALFAMQLASASVLPMALKSALELDLLEIMAKNGSPMSPTEIASKLPTKNPEAPVMLDRILRLLTSYSVLTCSNRKLSGDGVERIYGLGPVCKYLTKNEDGVSIAALCLMNQDKVLMESWYHLKDAILDGGIPFNKAYGMSAFEYHGTDPRFNKVFNNGMSNHSTITMKKILETYKGFEGLTSLVDVGGGIGATLKMIVSKYPNLKGINFDLPHVIEDAPSHPGIEHVGGDMFVSVPKGDAIFMKWICHDWSDEHCVKFLKNCYESLPEDGKVILAECILPETPDSSLSTKQVVHADCIMLAHNPGGKERTEKEFEALAKASGFKGIKVVCDAFGVNLIELLKKL

SEQ ID NO: 18 - protein, 363 aa, Synthetic construct
MGSTAETQLTPVQVTDDEAALFAMQLASASVLPMALKSALELDLLEIMAKNGSPMSPTEIASKLPTKNPEAPVMLDRILRLLTSYSVLTCSNRKLSGDGVERIYGLGPVCKYLTKNEDGVSIAALCLMNQDKVLMESWYHLKDAILDGGIPFNKAYGMSAFEYHGTDPRFNKVFNNGMSNHSTITMKKILETYKGFEGLTSLVDVGGGIGATLKMIVSKYPNLKGINFDLPHVIEDAPSHPGIEHVGGDMFVSVPKGDAIFMKFICHDWSDEHCVKFLKNCYESLPEDGKVILAECILPETPDSSLSTKQVVHADCIMLAHNPGGKERTEKEFEALAKASGFKGIKVVCDAFGVNLIELLKKL

SEQ ID NO: 19 - protein, 363 aa, Synthetic construct
MGSTAETQLTPVQVTDDEAALFAMQLASASVLPMALKSALELDLLEIMAKNGSPMSPTEIASKLPTKNPEAPVMLDRILRLLTSYSVLTCSNRKLSGDGVERIYGLGPVCKYLTKNEDGVSIAAICLMNQDKVLMESWYHLKDAILDGGIPFNKAYGMSAFEYHGTDPRFNKVFNNGMSNHSTITMKKILETYKGFEGLTSLVDVGGGIGATLKMIVSKYPNLKGINFDLPHVIEDAPSHPGIEHVGGDMFVSVPKGDAIFMKWICHDWSDEHCVKFLKNCYESLPEDGKVILAECILPETPDSSLSTKQVVHADCIMLAHNPGGKERTEKEFEALAKASGFKGIKVVCDAFGVNLIELLKKL

SEQ ID NO: 20 - protein, 363 aa, Synthetic construct
MGSTAETQLTPVQVTDDEAALFAMQLASASVLPMALKSALELDLLEIMAKNGSPMSPTEIASKLPTKNPEAPVMLDRILRLLTSYSVLTCSNRKLSGDGVERIYGLGPVCKYLTKNEDGVSIAAICLMSQDKVLMESWYHLKDAILDGGIPFNKAYGMSAFEYHGTDPRFNKVFNNGMSNHSTITMKKILETYKGFEGLTSLVDVGGGIGATLKMIVSKYPNLKGINFDLPHVIEDAPSHPGIEHVGGDMFVSVPKGDAIFMKWICHDWSDEHCVKFLKNCYESLPEDGKVILAECILPETPDSSLSTKQVVHADCIMLAHNPGGKERTEKEFEALAKASGFKGIKVVCDAFGVNLIELLKKL

SEQ ID NO: 21 - protein, 505 aa, Psychotria carthagenensis
MDSLDENNVSFSPPQLVTEFKPLDIQEFRTQAHRMVDFIADYYQNIENYPVLSQVEPGQLRNRLPESAPYRPEPFENILRDIKTSIIPGMTHWLSPNFFAFFPATVSSAAFVGEMLCTCFNAVGFNWLASPATTELEMVVMDWLAQLLMLPKSFMFSGSGGGVIQGTTSESILVALIAARDRALAVIGAHGISKLVVYGSDQTHSSFAKTCKVAGIFPCNIRSIPTTAENNFAFSPAVLRRVIEADVASGMVPLFICATIGTTSTTAIDPLSQLADVANDHGVWLHVDAAYGGSACICPEFRHYLDGIERVDSLSLSPHKWLLSYLDCCCLWVKQPNSLVKALSTNPEYLRNKMSELGSVVDFKDWQVGTGRRFKALRLWLIMRSYGVANLQSHIRSDVRMAKMFEEFINSDPRFEVVVPRTFSLVCFRFNPYKESDKAYINLLNKKLLESVNSTGRLYMTHTVAGDAFVLRFAVGATLTEDRHICAAWELIKESAEALLKNNVV

SEQ ID NO: 22 - protein, 507 aa, Psychotria carthagenensis
MDSLDENNVSFSPPQLVTEFKPLDIQEFRTQAHRMVDFIADYYQNIENYPVLSQVEPGQLRNRLPESAPYRPEPFENILRDIKTSIIPGMTHWLSPNFFAFFPATVSSAAFVGEMLCTCFNAVGFNWLASPATTELEMVVMDWLAQLLMLPKSFMFSGSGGGVIQGTTSESILVALIAARDRALAVIGAHGISKLVVYGSDQTHSSFAKTCKVAGIFPCNIRSIPTTAENNFAFSPAVLRRVIEADVASGMVPLFICATIGTTSTTAIDPLSQLADVANDHGVWLHVDAAYGGSACICPEFRHYLDGIERVDSLSLSPHKWLLSYLDCCCLWVKQPNSLVKALSTNPEYLRNKMSELGSVVDFKDWQVGTGRRFKALRLWLIMRSYGVANLQSHIRSDVRMAKMFEEFINSDPRFEVVVPRTFSLVCFRFNPYKESDKAYINLLNKKLLESVNSTGRLYMTHTVAGEAFVMGFAVGATLTEDRHICAAWELIKESAEALLKRSYYIV

SEQ ID NO: 23 - protein, 509 aa, Psychotria carthagenensis
MGTIDTNDGAYPDSIFTPFNPLDPEEFRKKAHIMVDFIADYYKNIENYPVLSQVEPGYLRTRLPKTAPYLPESFETILSDIQKDIIPGMTNWLSPNFFAFFPATVSSAAFLGEMLCTGFNSVGFNWLASPAATELEMVVMDWMAKMLKLPKTFMFSGTGGGVLQGTTSEAILCVLIAARDRAFERIGIQNIGKLVAYGSDQTHSFFQKTCKLAGIFPCNIKIIPTTREANFTLSPAALRRQIEADLTDGLVPLFLCATVGTTSTTAVDPVGELAKVANDFNVWVHVDAAYAGSACICPEFRQYLDGVERADSLSLSPHKWLLSYLDCCCLWVKEPDSMVKALSTNPEYLRNKRSEFGSVVDFKDWQIGTGRRFRALRLWLIMRSYGVANLQDHIRSDIQMAKMFEGFVRSDPRFELVVPRAFSLVCFRLKPMRESSEPYTELLNKNLLELVNSTGRAYMTHTKVGGTYMLRFTVGATLTEERHVCAAWELITECTDVLLKEFHLPGLPK

SEQ ID NO: 24 - protein, 509 aa, Psychotria carthagenensis; identical to SEQ ID NO: 23

SEQ ID NO: 25 - protein, 510 aa, Psychotria carthagenensis
MGTIDTNDGAYPDSIFTPFNPLDPEEFRKKAHIMVDFIADYYKNIENYPVLSQVEPGYLRTRLPKTAPYLPESFETILSDIQKDIIPGMTNWLSPNFFAFFPATVSSAAFLGEMLCTGFNSVGFNWLASPAATELEMVVMDWMAKMLKLPKTFMFSGTGGGVLQGTTSEAILCVLIAARDRAFERIGIQNIGKLVAYGSDQTHSFFQKTCKLAGIFPCNIKIIPTTREANFTLSPAALRRQIEADLTDGLVPLFLCATVGTTSTTAVDPVGELAKVANDFNVWVHVDAAYAGSACICPEFRQYLDGVERADSLSLSPHKWLLSYLDCCCLWVKEPDSMVKALSTNPEYLRNKRSEFGSVVDFKDWQIGTGRRFRALRLWLIMRSYGVANLQDHIRSDIQMAKMFEGFVRSDPRFELVVPRAFSLVCFRLKPMRESSEPYTELLNKNLLELVNSTGRAYMTHTKVGGTYMLRFTVGATLTEERHVCAAWELITECTDVLLKEFHLPVIRTI

SEQ ID NO: 26 - protein, 351 aa, Psychotria carthagenensis; identical to SEQ ID NO: 6